SEQUENCE LISTING (4 sequences)

SEQ ID NO: 1 - DNA, 20 bp, Bacteriophage T7
taatacgactcactataggg

SEQ ID NO: 2 - DNA, 2652 bp, Bacteriophage T7
atgaacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacagggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagagttcggcttccgtcaacaagtgctggaagatattattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttacagcctaccattaacaccaacaaagatagcgagattgatgcacacaaacaggagtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaa

SEQ ID NO: 3 - DNA, 3616 bp, Bacteriophage T7
atctcccgatcccctatggtgcactctcagtacaatctgctctagtgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgtatctgaggggactagggtgtgtttaggcgaaaagcggggcttcggttgtacgcggttaggagtcccctcaggatatagtagtttcgcttttgcatagggagggggaaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattccgcattgcagagatattgtatttaagtgcctagctcgatacaataaacgccatttgaccattcaccacattggtgtgcacctccaagcttacaaaacaaacaaacaactgtgtttacttgcaatcccccaaaacagacagaagcttccaccatgaacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcatcattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacagggctcgcaagtctcgccgtatcagccttgaggtcatgcttgagcaagcccataagtttgctaaccataaggccatctggttcccttacaacatggactggcgcggtcgtgtttacgcygtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttacagcctaccattaacaccaacaaagatagcgagattgatgcacacaaacaggagtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaatgaactagtaacggccgccagtgtgctggaattctgcagatatccatcacactggcggccgctcgagcatgcatctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtgggggggggcaggacagcaagggggaggattgggaagacaatagcaggcatgctgggga

SEQ ID NO: 4 - DNA, 51 bp, Artificial
  shRNA comprising 21 bases of the sense sequence of HBV mRNA from position 1907 to 1927 in the HBV genome
ttccgcagtatggatcggcagagagaacttctgccgatccatactgcggaa

(19) United States
(12) Patent Application Publication
(10) Pub. No.: US 2006/0183231 A1
(43) Pub. Date: Aug. 17, 2006

(54) Multiple-compartment eukaryotic expression systems

(76) Inventors: Catherine Pachuk, Lansdale, PA (US); Chandrasekhar Satishchandran, Lansdale, PA (US)

Correspondence Address: POTTER ANDERSON & CORROON LLP;ATTN: KATHLEEN W. GEIGER, ESQ., P.O. BOX 951, WILMINGTON, DE, 19899-0951, US

(21) Appl. No.: 11/359,145

(22) Filed: Feb. 22, 2006

Related U.S. Application Data
(63) Continuation-in-part of application No. PCT/US04/26999, filed on Aug. 20, 2004.
(60) Provisional application No. 60/497,304, filed on Aug. 22, 2003.

(51) Int. Cl. C12N 15/86; C12N 5/08

(52) U.S. Cl. 435/456; 435/366

(57) ABSTRACT

Method and constructs for expressing heterologous sequences of interest in eukaryotic cells using multiple-compartment expression systems. These systems, which may be comprised of a single construct or multiple constructs, utilize at least two different promoters which are each active within a different subcellular compartment of the same eukaryotic cell. The system and constructs of the invention are particularly useful for achieving enhanced in vivo expression of RNA molecules capable of modulating the expression of target genes.

### CROSS-REFERENCE TO RELATED APPLICATIONS

This application is a continuation-in-part of PCT Application No. PCT/US2004/026999, filed Aug. 20, 2004, now pending, which claims priority from U.S. Provisional Application Ser. No. 60/497,304 filed on Aug. 22, 2003, each of which is hereby incorporated by reference in their entireties.

### FIELD OF THE INVENTION

The present invention relates generally to the field of molecular biology and expression systems. Particularly, the invention relates to the expression of heterologous sequences of interest in eukaryotic cells using multiple-compartment expression systems, e.g., one or more expression constructs which collectively utilize at least two different promoters which are each active within a different subcellular compartment of the same eukaryotic cell.

### BACKGROUND OF THE INVENTION

Nucleic acids have come to be recognized as extremely valuable agents with significant and varied biological activities, including their use as therapeutic moieties in the prevention and/or treatment of disease states in man and animals. For example, oligonucleotides acting through antisense mechanisms are designed to hybridize to target mRNAs, thereby modulating the activity of the mRNA. Another approach to the utilization of nucleic acids as therapeutics is designed to take advantage of triplex or triple strand formation, in which a single-stranded oligomer (e.g., DNA or RNA) is designed to bind to a double-stranded DNA target to produce a desired result, e.g., inhibition of transcription from the DNA target. Yet another approach to the utilization of nucleic acids as therapeutics is designed to take advantage of ribozymes, in which a structured RNA or a modified oligomer is designed to bind to an RNA or a double-stranded DNA target to produce a desired result, e.g., targeted cleavage of RNA or the DNA target, thus inhibiting its expression. Nucleic acids may also be used as immunizing agents, e.g., by introducing into the tissues or cells of an organism DNA molecules that express proteins capable of eliciting an immune response. Nucleic acids may also be engineered to generate RNA that is translated to produce protein(s) that have biological function.

More recently, the phenomenon of RNAi or double-stranded RNA (dsRNA)-mediated gene silencing has been recognized, whereby dsRNA complementary to a region of a target gene in a cell or organism inhibits expression of the target gene (see, e.g., WO 99/32619, published 1 Jul. 1999, Fire et al.; and U.S. Pat. No. 6,506,559: “Genetic Inhibition by Double-Stranded RNA”; WO 00/63364: “Methods and Compositions for Inhibiting the Function of Polynucleotide Sequences,” Pachuk and Satishchandran; and U.S. Ser. No. 60/419,532, filed Oct. 18, 2002). dsRNA gene silencing presents a particularly exciting potential application for nucleic acid-based technology. Double-stranded RNA has been shown to induce gene silencing in a number of different organisms. (See e.g., Li et al., demonstrating dsRNA gene silencing in widely divergent vertebrates, i.e., zebrafish, avian tissue, and mammalian tissue culture; US2002/0114784A1, pub. 22 Aug. 2002). Gene silencing can occur through various mechanisms, one of which is post-transcriptional gene silencing (PTGS). In post-transcriptional gene silencing, transcription of the target locus is not affected, but the RNA half-life is decreased. Exogenous dsRNA has been shown to act as a potent inducer of PTGS in plants and animals, including nematodes, trypanosomes, insects, and mammals. Transcriptional gene silencing (TGS) is another mechanism by which gene expression can be regulated. In TGS, transcription of a gene is inhibited. The potential to harness dsRNA mediated gene silencing for research, therapeutic, and prophylactic indications is enormous. The exquisite sequence specificity of target mRNA degradation and the systemic properties associated with PTGS make this phenomenon ideal for functional genomics and drug development.

Some current methods for using dsRNA in vertebrate cells to silence genes result in undesirable non-specific cytotoxicity or cell death due to dsRNA-mediated stress responses, including the interferon response. Induction of a dsRNA-mediated stress response is rapid, and may result in cellular apoptosis or anti-proliferative effects. In addition to the potential for dsRNA to trigger toxicity in vertebrate cells, dsRNA gene silencing methods may result in non-specific or inefficient silencing. It has become dogma in the RNAi field that dsRNA molecules greater than 30 bps in length may not be used in adult mammals because of a stress or “panic” response. Applicants have demonstrated, however, that intracellular expression of dsRNA, including the long dsRNAs reported to induce toxicity in vertebrate cells, can be accomplished under conditions which do not trigger dsRNA-mediated toxicity. See, US2002/2132257A1, published 19 Sept. 2002, showing that intracellular expression of long dsRNAs does not induce a type I interferon response (RNA stress response) in stress-response capable mammalian cells. No evidence of dsRNA stress response induction was detected from intracellularly expressed long dsRNAs (e.g., 600 bp) as measured by: TUNEL assay to detect apoptotic cells, ELISA assays to detect the induction of alpha, beta and gamma interferon, ribosomal RNA fragmentation analysis to detect activation of 2′5′ OAS, measurement of phosphorylated eIF2a as an indicator of PKR (protein kinase RNA inducible) activation, proliferation assays to detect changes in cellular proliferation, and microscopic analysis of cells to identify cellular cytopathic effects. In contrast, poly(I)(C)RNA as well as in vitro transcribed 600 bp dsRNA transfected into the same cells induced an RNA stress response. Accordingly, methods and compositions providing for intracellular expression of dsRNAs from dsRNA expression constructs hold out great promise for therapeutic applications in mammals and other vertebrates. However, a challenge remains in that the practical implementation of such dsRNA methods requires the efficient intracellular production and delivery of dsRNA from dsRNA expression constructs.

For all these mechanisms of biological activity, it is frequently desirable to express a biologically active nucleic acid intracellularly from a nucleic acid expression construct. The effectiveness of such methods depends upon an ability to efficiently express the selected nucleic acid in the target host cell in a therapeutically relevant manner, e.g., in a biologically active, non-toxic form to the desired target cell or cells in vivo or in vitro, in effective amounts and duration in the desired subcellular location or location(s). This presents a particular challenge in cells which are difficult to transfect, e.g., primary cells, certain cell lines, e.g., K5625, a human leukemia cell line, and for in vivo applications. Thus, improved expression systems, expression constructs, and methods are needed for intracellular expression of nucleic acids from nucleic acid expression constructs in eukaryotes. Desirably, these methods may be used to provide nucleic acids capable of achieving any of their varied biological functions, including production of a desired polypeptide and/or a desired therapeutic RNA, e.g., a ribozyme, antisense, triplex-forming, and/or dsRNA in in vitro samples, cell culture, tissue or organ explant, and intact animals (e.g., vertebrates, such as mammals, including humans).

In the decades since the advent of biotechnology, a huge variety of vectors, expression constructs, and expression systems, including circular plasmids, linearized plasmids, cosmids, phage vectors, viral genomes, recombinant viral genomes, artificial chromosomes, etc., have been developed for use in prokaryotes and/or eukaryotes. Use of these expression systems in bacterial cell culture has made such recombinant proteins as interferon (alpha), interferon (beta), erythropoietin, factor VIII, human insulin, t-PA, and human growth hormone a standard part of the pharmaceutical armamentarium.

Among the tremendous variety of expression vectors and expression systems that have been developed in the field of biotechnology and molecular biology are expression systems containing multiple promoters on the same vector. One such type of multiple promoter expression system utilizes vectors containing multiple promoters (i.e., two or more promoters) that are active in a prokaryote or in the same subcellular compartment of a eukaryotic cell. For example, such multiple promoter systems in the art have been developed to permit expression of more than one sequence in the same compartment of the same cell (e.g., two distinct sequences or a sense and antisense sequence designed to form a dsRNA), or they may be used to express the same sequence within different cells or organisms (e.g., a prokaryote and a eukaryote) or to obtain more efficient transcription of a single operably linked sequence. Frequently seen are, e.g., multiple RNA polymerase II promoters or bacteriophage promoters on the same plasmid, such as, e.g., a bacteriophage T7 promoter and a bacteriophage SP6 promoter (each of which is active in the cytoplasm of a eukaryotic cell if supplied with the cognate polymerase). Such plasmid vectors which utilize bacteriophage promoters such as T7 to express various transcripts will also commonly include a polymerase II promoter such as CMV or SV40 for expression of a protein such as a selection marker (e.g., an antibiotic resistance gene) or a reporter gene.

Further, such multiple promoters can be arranged within the vector in any number of orientations and configurations. For example, promoters can be arranged divergently with respect to each other, in which case, they drive transcription in the same direction within the vector. Alternatively, multiple promoters may be arranged convergently with respect to each other in the same vector, in which case, transcription proceeds in opposite directions within the vector. Further, a variety of terms have been developed in the art to describe the relative position of multiple promoters within a single vector. The term “tandem” has been used to describe multiple promoters that all reside on, and are all operably linked to, the 5′ end of the sequence to be transcribed. Tandem promoters can be the same or different promoters. The term “flanking” promoters describes the orientation of multiple promoters in which the sequence to be transcribed is flanked on both the 5′ and the 3′ end by a promoter in such a manner that each promoter, when transcriptionally active, is capable of transcribing one strand of the sequence to be transcribed. The flanking promoters can be the same or different promoters. For example, a set of bacteriophage T7 RNA polymerase promoters flanking the 5′ and 3′ ends of a sequence is a common method for expressing sense and antisense strands to form duplex double-stranded RNA (dsRNA) (WO99/32619, Fire et al., published Jul. 1, 1999).

Multiple tandem promoters are described, e.g., in U.S. Pat. No. 5,547,862, which discloses a DNA vector which comprises an RNA transcription sequence positioned downstream from two or more tandem promoters which are recognized by distinct RNA polymerases and are each capable of promoting expression of the RNA transcription sequence. A vector in this disclosure, for example, is a plasmid encoding the bacteriophage SP6, T7 and T3 promoters, each positioned upstream of and operably linked to a cloning site capable of accepting an RNA transcription sequence.

A method for making mammalian collagen or procollagen in yeast is disclosed in U.S. Pat. No. 6,472,171 using a construct comprising, in opposite orientations, two mammalian collagen genes operably linked to a single or dual, divergent heterologous promoter(s). The promoter(s) driving the two collagen genes may be the same promoter, or different promoters, and may be used to provide for the coordinate, preferably simultaneous, expression of the two collagen genes.

Expression vectors containing dual bacterial promoters arranged in tandem and operably linked to a heterologous nucleic acid encoding a desired polypeptide are disclosed in U.S. Pat. No. 6,117,651. The dual promoter comprises a first component derived from a tac-related promoter (which is itself a combination of the lac and trp promoters) and a second promoter component obtained from a bacterial gene or operon that encodes an enzyme involved in galactose metabolism. The dual bacterial promoter system acts synergistically to provide a high level of transcription of the linked sequence in a prokaryotic cell such as E. coli.

U.S. Pat. No. 5,874,242 discloses a vector which provides for the translation of an inserted coding sequence in both eukaryotic and prokaryotic host cells. Specifically, such vectors include either a bifunctional promoter (functional in both eukaryotes and prokaryotes) or dual promoters (promoters separately functional in eukaryotes and prokaryotes) for efficient expression in both prokaryotic and eukaryotic cells.

There are a myriad of other examples in the art disclosing variations on themes of multiple promoters used in the same vector. There remains, however, a need for more efficient expression systems particularly adapted to be active in more than one compartment of eukaryotic cells having multiple subcellular compartments. Intracellular expression of nucleic acids in eukaryotes, including nucleic acids designed to be translated into proteins, presents significant new challenges. With nucleic acid-based compositions showing such promise for pharmaceutical applications, e.g., for DNA vaccines and for dsRNAs and antisense moieties for modulation of nucleic acid expression, it is of critical importance to develop methods for more efficient RNA expression in eukaryotic cells. This is especially true for in vivo delivery applications because there are no efficient systems for DNA uptake into cells, and for primary cells and cell lines which are difficult to transfect.

### SUMMARY OF THE INVENTION

In general, the invention relates to novel nucleic acid expression systems, expression constructs, methods for generating them, and methods of utilizing them to make biologically active nucleic acids, and, if desired, polypeptides. More particularly, the invention relates to methods and compositions for expression of nucleic acids (e.g., DNA, RNA, hybrid, heteroduplex, and modified nucleic acids) in a eukaryotic cell, plant, or animal (e.g., a mammal, such as a human). The nucleic acid expression systems and expression constructs of the invention permit biologically active nucleic acids to be efficiently expressed in eukaryotic cells and organisms in vitro and in vivo in a manner and form that allows the nucleic acids to carry out their desired biological functions. Notably, the nucleic acid expression systems function efficiently in eukaryotic cells regardless of their sub-cellular localization.

More particularly, the invention provides multiple-compartment eukaryotic expression systems comprising one or more expression constructs, wherein the construct or constructs collectively which comprise the system will include at least two different promoters, including at least two promoters each active within a different subcellular compartment of the same eukaryotic cell. The multiple-compartment eukaryotic expression systems may include multiple expression constructs or a single expression construct, which include two or more different promoters, including at least two promoters each transcriptionally active in a different subcellular compartment, e.g., the cytoplasm, the mitochondria, the nucleolus, the nucleus (non-nucleolar), and functional domains within a particular subcellular compartment of the same eukaryotic cell. The multiple compartment expression system will include at least two promoters selected from at least two of a polymerase I promoter, a polymerase II promoter, a polymerase III promoter, a cytoplasmic promoter, and a mitochondrial promoter. In one aspect, the expression construct comprises such at least two different promoters operably linked to a sequence encoding a therapeutic RNA molecule, i.e., an antisense RNA, a ribozyme, a triplex forming RNA, an aptamer RNA, or a dsRNA molecule. In one aspect, the expression construct comprises such at least two different promoters operably linked to a sequence encoding a dsRNA molecule.

### BRIEF DESCRIPTION OF THE SEQUENCES

SEQ ID NO:1 represents a T7 promoter.

SEQ ID NO:2 represents the T7 RNA polymerase gene.

SEQ ID NO:3 represents a T7 RNA polymerase expression unit comprising the RSV promoter, the 5′ UTR, the T7 RNA polymerase coding region, and the BGH polyadenylation site.

SEQ ID NO:4 represents HBV shRNA 1907.

### BRIEF DESCRIPTION OF THE DRAWINGS

FIG. 1 is a schematic illustration of a plasmid expression vector which contains the same sequence under the control of two or more promoters. At least two promoters are used, each active in a different physical subcellular compartment and/or a separate functional domain of a subcellular compartment, so that there is a higher likelihood of the sequence being transcribed regardless of the subcellular environment to which the vector localizes following transfection in vitro or in vivo. For example, the plasmid of FIG. 1A includes one copy of Sequence A (which encodes a hairpin dsRNA), operably linked to a T7 promoter (T7p), and a second copy of Sequence A, under the control of an RNA pol III promoter, such as the human U6 promoter (U6p). Each transcription unit includes the appropriate terminator sequence, T7t and U6t, respectively. In FIG. 1A, the promoters are divergent with respect to each other (i.e., transcription proceeds in the same direction). In FIG. 1B, the T7 promoter and the U6 promoter flank the encoded dsRNA Sequence A and are convergent with respect to each other. (Terminators are not shown but would be arranged as in FIG. 8.) The arrows denote the direction of transcription.

FIG. 2 illustrates a “flanked promoter arrangement” in which a single sequence is flanked on each end by a promoter in such a way that each promoter when transcriptionally active is capable of transcribing one strand of said sequence. P1 and P2 represent two different promoters, each transcriptionally active in a different subcellular compartment of a single eukaryotic cell or a different functional domain of a single subcellular compartment. The sequence of interest encodes, e.g., an RNA hairpin or an mRNA.

FIG. 3 illustrates a triple promoter construct in which two copies of a selected Sequence A are arranged so that one copy of the desired RNA coding region is flanked on one end by a T7 promoter and on the other end by a U6 promoter. (Terminators are not shown.) A second copy of the encoded RNA sequence located elsewhere in the vector is under the control of an RNA pol II promoter such as the MCMV (murine cytomegalovirus) immediate early promoter or HCMV (human cytomegalovirus) immediate early promoter.

FIG. 4 illustrates a plasmid expression vector with embedded HCMV and T7 promoters located in tandem at the 5′ end of the T7 RNA polymerase gene. (Terminator and polyadenylation site are not shown.)

FIG. 5 illustrates three promoters, P1, P2, and P3, arranged in tandem at the 5′ end of the sequence of interest. They differ from each other in terms of the cellular compartment in which they are transcriptionally active.

FIG. 6 illustrates a plasmid expression construct which utilizes the RNA pol II promoter HCMV and the cytoplasmic T7 promoter in tandem to drive the sequence of interest. Nuclear transcripts from the HCMV promoter will include a 5′ cap, a T7 promoter sequence, and the sequence of interest. Cytoplasmic transcripts from the T7 promoter will include only the sequence of interest.

FIG. 7 is a schematic illustration of a plasmid expression vector with four separate cistrons, as described in greater detail in Example 1. There are two copies of Sequence A and two copies of Sequence B, each encoding a different hairpin dsRNA, including inverted sense and antisense HBV sequences separated by a “loop” region. One copy of Sequence A is operably linked to a bacteriophage T7 promoter (T7p) and a T7 terminator (T7t), and the other copy is operably linked to a human U6 promoter (U6p) and a U6 terminator (U6t). One copy of Sequence B is operably linked to a bacteriophage T7 promoter (T7p) and a T7 terminator (T7t), and the other copy is operably linked to a human U6 promoter (U6p) and a U6 terminator (U6t). The arrow denotes the direction of transcription.

FIG. 8 is a schematic illustration of a bicistronic plasmid expression vector (described in greater detail in Example 2) with a flanked promoter arrangement: two different subcompartment specific promoters, bacteriophage T7 (T7p) and human U6 (U6p), flank and are operably linked to a sequence encoding an HBV (hepatitis B virus)-specific hairpin dsRNA (Sequence A). “T7t” denotes the T7 terminator and “U6t” denotes the U6 terminator. The arrow indicates the direction of transcription. The T7 transcript will contain, from 5′ to 3′, the reverse complement to the U6 terminator, the Sequence A hairpin. The U6 transcript will contain from 5′ to 3′ the reverse complement to the T7 terminator, Sequence A hairpin.

FIG. 9 is a schematic illustration of a bicistronic plasmid expression vector (described in greater detail in Example 5) in which each cistron occupies a physically separate location on the plasmid. Each cistron includes a copy of Sequence A, an HBV sequence, operably linked to a promoter so that only an antisense RNA is transcribed. The two cistrons are driven by different subcompartment-specific promoters. One cistron includes the bacteriophage T7 promoter (T7p) and T7 terminator (T7t), and the other cistron includes the MCMV and the BGH (bovine growth hormone) polyadenylation site. The arrow denotes the direction of transcription.

FIG. 10a is a schematic illustration of a plasmid expression vector (described in greater detail in Example 6) utilizing three promoters arranged in three cistrons: a nuclear promoter (RSV (Rous sarcoma virus)) to drive expression of the bacteriophage T7 RNA polymerase and two cytoplasmic T7 promoters, one expressing an HBV-specific sense RNA and one expressing an HBV-specific antisense RNA. The RSV promoter is paired with a BGH polyadenylation site, and a T7 terminator is positioned at the end of each T7 cistron. The arrows indicate the direction of transcription. The sense and antisense RNAs will be able to hybridize with each other to form a duplex dsRNA.

FIG. 10b is the sequence of the T7 RNA polymerase expression cassette utilized in the plasmid expression vector of FIG. 10a, and Example 6, comprising the RSV promoter, the 5′ UTR, the T7 RNA polymerase coding region, and the BGH polyadenylation site.

FIG. 11 is a schematic representation of a plasmid expression vector with a Dual/Embedded Promoter system. The bacteriophage T7 promoter replaces the 17 nucleotides at the 3′ terminus of the MCMV promoter, so that the 5′ nucleotide of the T7 promoter is adjacent to nucleotide 1123 of the MCMV promoter. The MCMV promoter and the T7 promoter are each operably linked to an HBV-specific antisense sequence, Sequence A. A T7 terminator (T7t) and a BGH polyadenylation site are positioned 3′ to Sequence A as shown. When the expression construct is located in the cytoplasm, the T7 promoter initiates transcription of Sequence A, whereas expression constructs localized in the nucleus will express the antisense RNA from the MCMV promoter, which is a RNA pol II promoter.

FIG. 12 depicts the sequence of the dual/embedded promoter described in Example 7 and FIG. 11. The truncated MCMV promoter (nts 1-1123) is placed so that nt 1123 directly abuts the most 5′ nucleotide of the T7 promoter. The arrows designate the direction of transcription.

### DETAILED DESCRIPTION

Applicants specifically incorporate the entire content of all cited references in this disclosure. Further, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the invention be limited to the specific values recited when defining a range.

The realization of the tremendous promise of biologically active nucleic acids, including nucleic acid-based pharmaceuticals, particularly for in vivo applications in eukaryotes, depends to a large extent upon our ability to develop more efficient delivery and expression systems. Applicants' invention is directed to multiple-compartment eukaryotic expression systems designed for the efficient expression of heterologous nucleic acid sequences in eukaryotic cells. Particularly, the invention relates to methods for expression of sequences of interest in eukaryotic cells using multiple-compartment eukaryotic expression systems, e.g., one or more expression constructs which utilize at least two different promoters, wherein at least two different promoters are each transcriptionally active within a different subcellular compartment of the same eukaryotic cell. The multiple compartment-specific promoters may be located on a single expression vector or expression construct, e.g., a plasmid, recombinant virus, etc., or they may be located on different expression constructs within a single composition, or on different expression constructs located within a single eukaryotic cell. In using the term “multiple promoters”, applicants specifically mean more than one different promoter sequence. Such different promoter sequences are each active within a different subcellular compartment of a single eukaryotic cell. It is anticipated that the expression systems of the invention may utilize more than one copy of the same promoter (e.g., two T7 promoters), and/or more than one promoter active in the same subcellular compartment (e.g., a T7 promoter and a SP6 promoter), so long as the expression system as a whole includes at least two promoters active in different subcellular compartments (e.g., a T7 promoter and a human mitochondrial light chain promoter). It is also contemplated that the expression systems of the invention may utilize promoter combinations which include two or more promoters active in different domains present within the same structural subcellular compartment, i.e., two promoters transcriptionally active within different domains of the nucleus.

Accordingly, the expression systems of the invention will comprise at least two promoters each active in a different physical subcellular compartment and/or a separate functional domain of a subcellular compartment of a eukaryotic cell, i.e., at least two promoters selected from at least two of: a polymerase I promoter, a polymerase II promoter, a polymerase III promoter, a cytoplasmic promoter, and a mitochondrial promoter. Similarly, combinations of polymerase I and polymerase II promoters; polymerase. I and polymerase III promoters; polymerase II and polymerase III promoters; polymerase I and mitochondrial promoters; polymerase II and mitochondrial promoters; polymerase III and mitochondrial promoters; polymerase I and cytoplasmic promoters; polymerase II and cytoplasmic promoters; polymerase III and cytoplasmic promoters; as well as combinations of any of the preceding with promoters from still other classes, including all the various permutations of two, three, four, and five promoters selected from RNA polymerase I, RNA polymerase II, RNA polymerase III, mitochondrial, and cytoplasmic promoters, may be used as described. In one aspect, the expression construct comprises such at least two different promoters operably linked to a sequence encoding a therapeutic RNA molecule, i.e., an antisense RNA, a ribozyme, a triplex forming RNA, an aptamer RNA, or a dsRNA molecule. In one aspect, the expression construct comprises such at least two different promoters each operably linked to a sequence(s) encoding a dsRNA molecule.

Although generally applicable to many eukaryotic systems for the expression of a variety of sequences of interest, Applicants' multiple-compartment eukaryotic expression systems are particularly described herein as they relate to the efficient intracellular expression of biologically active RNA molecules (optionally translated into polypeptides). In one aspect, the expressed RNA molecules are biologically active therapeutic RNAs which are not translated into polypeptides. An important aspect of the invention relates to the ability to express more nucleic acid (and optionally polypeptide) per cell. Another important aspect of the invention relates to the ability to modulate biological activity by directing expression of nucleic acids in two, three, four or more subcellular compartments, as desired.

The application of the invention to the efficiency of nucleic acid expression systems in eukaryotes is particularly useful for several reasons. Following transfection of eukaryotic cells or in vivo delivery of nucleic acid expression constructs to eukaryotes, it is recognized that the expression construct distributes mainly to the cytoplasm and functional compartments therein, with a much smaller proportion localizing to the nucleus and nuclear compartments such as the nucleolus. The expression constructs are therefore dispersed in a very non-uniform manner within different subcellular compartments; moreover, the dispersion is not static (i.e., some cytoplasmic nucleic acid can eventually enter the nucleus and some nuclear nucleic acid can eventually localize to the cytoplasm). This results in a situation where populations of expression constructs (frequently, the majority of the expression constructs which make it into the cell) are non-functional simply because they are located in subcellular compartments in which the encoded promoters are not active. For example, promoters, including the widely used HCMV (human cytomegalovirus) immediate early promoter, which are driven by RNA polymerase II (RNA pol II), are active only in the nucleus but not in the cytoplasm where the greatest number of the expression constructs are located. The majority of such expression constructs in the cell (those in the cytoplasmic compartments) are therefore not active. By including two or more, e.g., several, promoters each active in a different subcellular compartment of a eukaryote, it is possible to engineer a multi-compartment eukaryotic expression system, e.g. a plasmid or combination of plasmids, that are transcriptionally active no matter where in the cell the plasmid(s) is localized. In some aspects, a single expression construct can be designed to be transcriptionally active in e.g., two, three, four, or even all subcellular compartments of a eukaryotic cell in which transcription occurs, or can be made to occur. In other aspect of the invention, a eukaryotic expression system comprising two or more expression constructs can be designed to include a combination of different-subcompartment promoters to be transcriptionally active in e.g., two, three, four, or even all subcellular compartments, including functional domains, within a single subcellular compartment, of a eukaryotic cell in which transcription occurs, or can be made to occur.

This issue of inefficient expression in eukaryotic cells can be important for in vitro applications, particularly in certain cells, such as primary cells, and in certain hard-to-transfect cell lines; it is critical, however, for in vivo applications where nucleic acid delivery tends to be relatively poor. Notably, in vivo delivery methods do not provide for efficient uptake of DNA into cells. For example, only about 10⁶-10⁷ DNA molecules are internalized into cells following injection of as many as 10¹⁴ molecules. This results in a situation in which not only are very few cells transfected, but also the cells that are transfected have only one to at most a few molecules of transfected DNA. Unless the DNA is in an appropriate sub-cellular compartment, it will not be expressed. The odds of being in the correct compartment go down as the number of transfected molecules per cell is decreased. Therefore a system in which multiple promoters are used and wherein the promoters are individually active in different sub-cellular compartments increases the likelihood that any cell that is transfected will be able to express the desired RNA. A similar situation exists for many in vitro applications including the transfection of primary cells which are difficult to transfect and certain cell lines, e.g., K562 cells, which are also difficult to transfect.

The use of multiple different subcompartment-specific promoters provides an additional advantage when the induction of both PTGS and TGS is desired. In these circumstances, expression of dsRNA needs to occur at least a minimal level in the cytoplasm and the nucleus and the nucleolus.

The multi-compartment eukaryotic expression systems and the methods of the invention present an opportunity to significantly increase the likelihood of expression of every plasmid taken up by a cell and a way of getting significantly more expression per transfected cell, e.g., an increase in the level of expression by up to about 50%, 100%, 200%, 500%, 1000%, 5000%, and even potentially up to 10,000% or more. Since expression constructs such as plasmids are typically distributed very unevenly within a eukaryotic cell, with 99% or more located in the cytoplasm and less than 1% (frequently much less than 1%) located within the nucleus, adding a cytoplasmic transcription capability can be expected to potentially increase expression 10 to 1000 fold or more.

Vectors containing multiple promoters that are active in the same subcellular compartment are commonly used. For example, multiple RNA pol II promoters are used, or, multiple bacteriophage promoters, e.g., a T7 and an SP6 promoter (each of which is active in the cytoplasm). Most of these known expression systems are designed to express multiple sequences. Some known expression constructs contain pol II promoters linked to a reporter gene or selection marker and a different polymerase promoter, such as T7, for transcribing a transcript of interest. Such known expression constructs have a number of limitations and drawbacks: These methods do not enable an expression construct to transcribe an RNA sequence of interest, in particular a therapeutic or regulatory RNA, in a eukaryotic cell independent of its subcellular compartmentalization. In addition, the use of two or more promoters active in the same compartment at the same time and present on the same nucleic acid molecule is likely to be inefficient and undesirable for a number of reasons:

a) Transcription from a DNA molecule affects the supercoiling of the template DNA ahead of the growing strand and behind the just transcribed strand (Marietta Dunaway and Elaine A. Ostrander, Nature 361:746-748 (1993); Jocelyn E. Krebs and Marietts Dunaway, Mol. Cell. Biol. 16:5821-5829 (1996)). These changes in supercoiling affect the entire plasmid. Changes in supercoiling detrimentally affect the activity of many promoters and can in fact abolish activity. This means that activity from one promoter in an expression construct can negatively impact on the activity of another promoter and vice versa.

b) Multiple promoters in a single expression construct, when both are active in the same compartment and running in the same direction, can result in promoter occlusion or promoter interference. Promoter interference occurs when promoters are situated near each other (within several hundred nucleotides). The nucleation of transcription factors and other factors on one promoter sterically hinders the nucleation of factors on the second promoter. Promoter occlusion is particularly a problem for systems in which a terminator is not located at the end of one cistron and before the next promoter. Since RNA pol II has no efficient termination system, this is a potential problem for the use of multiple RNA pol II promoters on the same expression construct. Promoter occlusion results when transcription from one cistron does not terminate at the end of the cistron and runs through a second promoter preventing transcription initiation from that promoter.

c) Transcription interference occurs when two active promoters both active in the same subcellular compartment are facing each other in the converging direction. Also transcription interference can occur in which a downstream promoter is repressed by the presence of an upstream promoter (both of which should be active at the same time on the same molecule). The extending transcript initiated from the upstream promoter represses initiation from the downstream promoter as the extending transcripts transverse the downstream promoter. (Proudfoot, N.J., Nature 322:562-565 (1986)). Transcription from one promoter can interfere with transcription from the other promoter and cause premature termination of the transcript.

The multi-compartment eukaryotic expression systems of the invention may advantageously be used to express one or more sequences of interest in two or more distinct subcellular compartments and/or distinct functional domains of a eukaryotic cell under the control of two or more different subcellular compartment-specific promoters. In one aspect of the invention, a first sequence is expressed in a first selected subcellular compartment and a second sequence is expressed in a second subcellular compartment, wherein the first and second compartments are different subcellular compartments of a single eukaryotic cell.

Dual or multiple promoters can be used in a single expression construct or in an expression system comprising two or more expression constructs to generate a system comprised of two or more transcriptional cistrons. Conceptually, the constructs may be devised, e.g, according to the following general principles:

Class 1. These vectors contain the same nucleic acid sequence under the control of two or more promoters. In one aspect a sequence encoding the same dsRNA is placed under the control of two promoters. Each promoter is active in a separate functional domain and/or physical subcompartment of a eukaryotic cell. This increases the likelihood of the sequence being transcribed regardless of which subcellular environment to which the vector localizes following transfection in vitro or in vivo administration and may result in more RNA being made per cell. An appropriate selection of promoters may also increase the likelihood of achieving the desired biological activity(ies) by focusing transcriptional activity in the requisite subcellular location(s).

One problem with current plasmid vector systems is that following transfection of a cell in vitro or in vivo, multiple copies of a plasmid/vector enter a cell, but not all plasmids/vectors co-localize to the same subcellular compartment and therefore not all plasmid/vector molecules containing a single type of promoter will be transcribed. A plasmid/vector capable of being transcribed in more than one subcellular compartment will allow a higher percentage of transfected plasmids/vectors to be expressed.

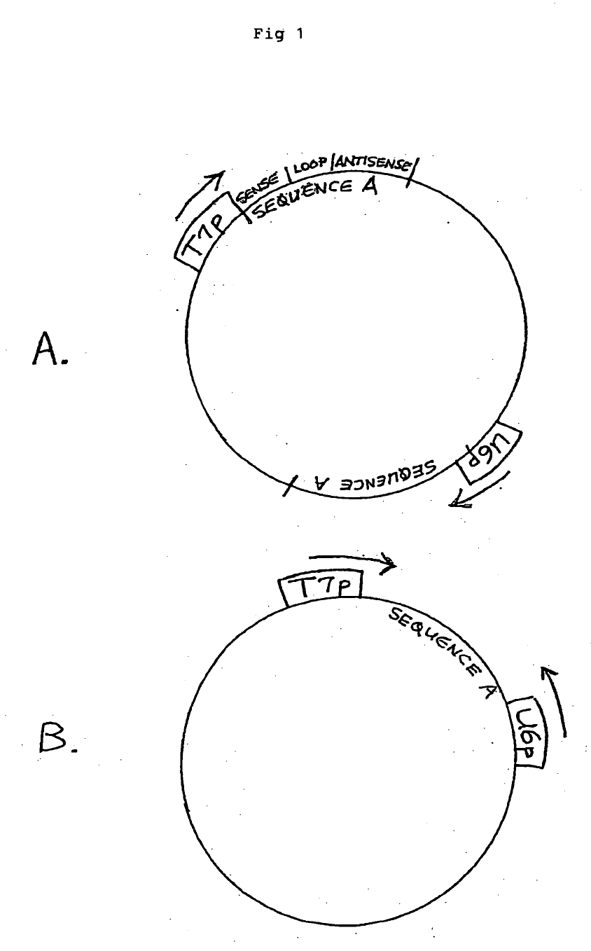

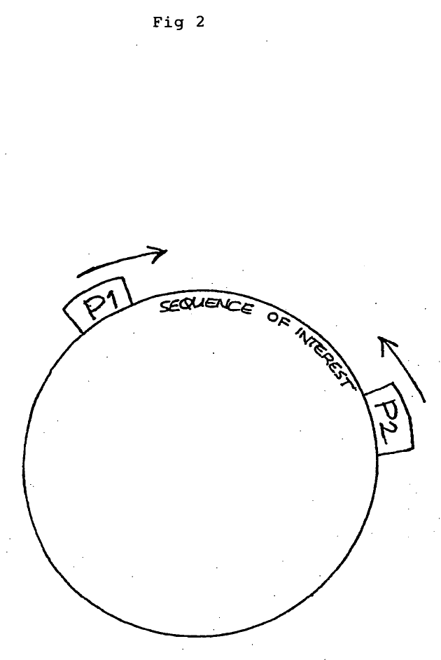

An example of a plasmid with two types of promoters is shown in FIG. 1A. One copy of Sequence A (encoding a dsRNA hairpin having inverted sense and antisense regions separated by a loop region) could be under the control of a T7 promoter (T7p) while a second copy of the Sequence A hairpin could be under the control of an RNA pol III promoter, such as the U6 promoter. (Terminators are not shown, but would be positioned as in FIG. 7.) When the vector localizes to the cytoplasmic compartment of a cell expressing T7 RNA polymerase, the T7 promoter driven cistron is transcribed. Transcription units driven by RNA pol III promoters will not be transcribed during the time they are in the cytoplasmic compartment. However, those vectors that localize to the nucleolus will be transcribed via the U6 promoter and not the T7 promoter.

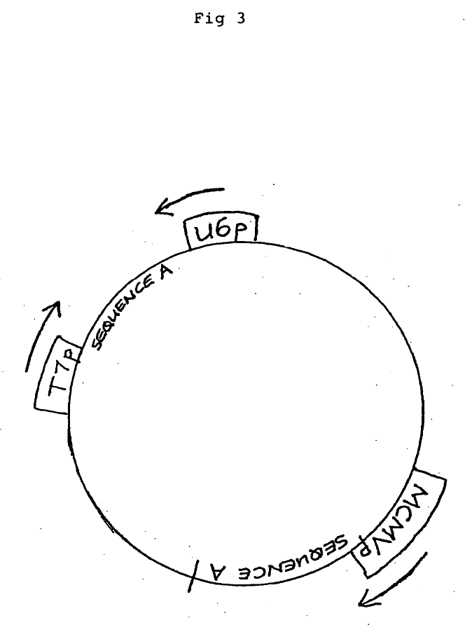

Alternatively one copy of the Sequence A hairpin could be flanked on one end by the T7 promoter and on the other end by the U6 promoter (FIG. 1B). (Terminators are not shown but would be arranged as in FIG. 8.) Vectors located in the cytoplasm will be transcribed from the T7 promoter end, while vectors in the nucleolus will be transcribed from the U6 promoter end. Such multi-compartment expression systems which utilize promoters transcriptionally active in different subcellular compartments enable the cell to make more of a specific hairpin RNA. If the RNA is designed to make protein, more of the protein will be made per cell. Compositions in which a single sequence is flanked on each end by a promoter in such a way that each promoter when transcriptionally active is capable of transcribing one strand of said sequence will be referred to as having a “flanked promoter arrangement”, as depicted in FIG. 2. P1 and P2 represent two different promoters, each transcriptionally active in a different subcellular compartment or a different functional domain of a single subcellular compartment. The sequence of interest encodes, e.g., an RNA hairpin or an mRNA.

A triple promoter combination can also be used, as, for example, in an expression construct which encodes one or more nucleic acids of interest under the control of, e.g., a T7 promoter, an RNA pol III promoter, and an RNA pol II promoter. Such a vector can be designed in which a single copy of the desired RNA coding region is flanked on one end by a T7 promoter and on the other end by a U6 promoter. A second copy of the encoded RNA sequence located elsewhere in the vector is under the control of an RNA pol II promoter such as the MCMV or HCMV promoter (see FIG. 3). Alternatively, three copies of the same sequence can be included in the vector, with each sequence under the transcriptional control of a separate promoter. In other embodiments, two or three different sequences may be arranged under the control of a triple promoter combination.

The foregoing are only illustrative examples of the multi-compartment eukaryotic expression systems of the invention and many additional embodiments can readily be envisioned by one of ordinary skill in the art of molecular biology.

Principles to consider with respect to Class 1 vectors:

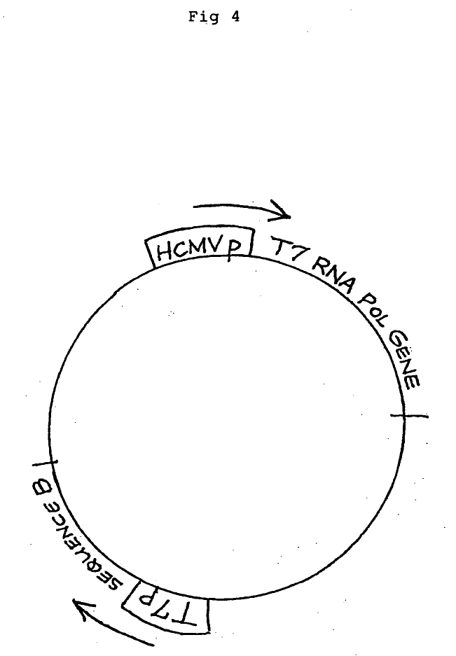

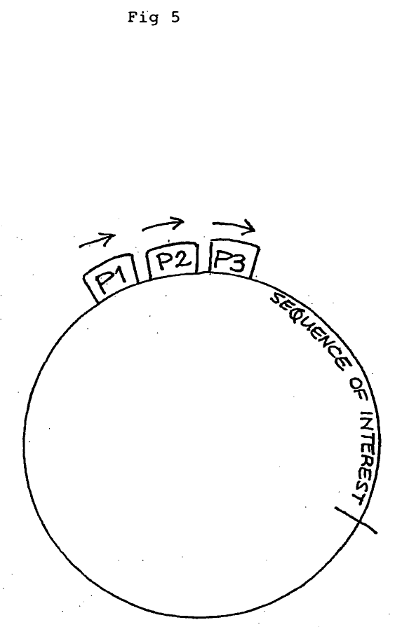

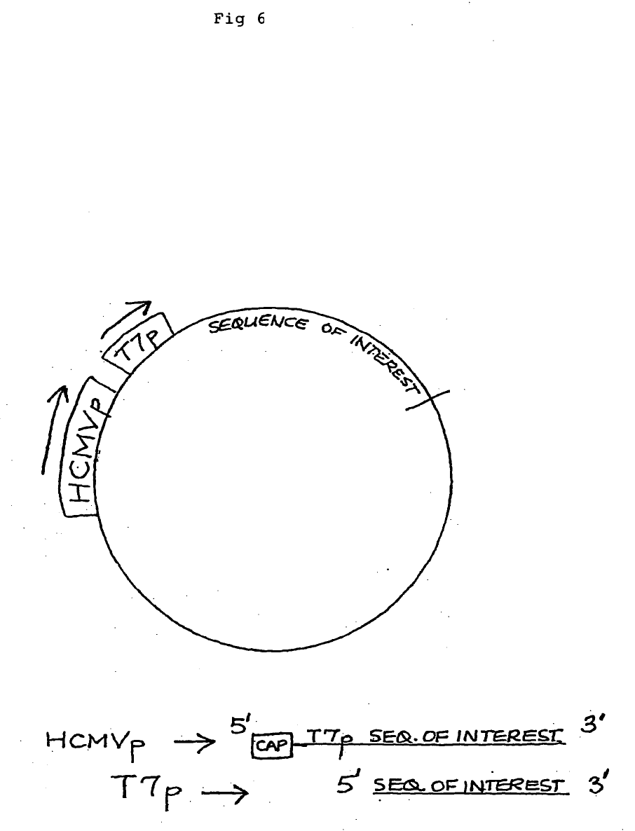

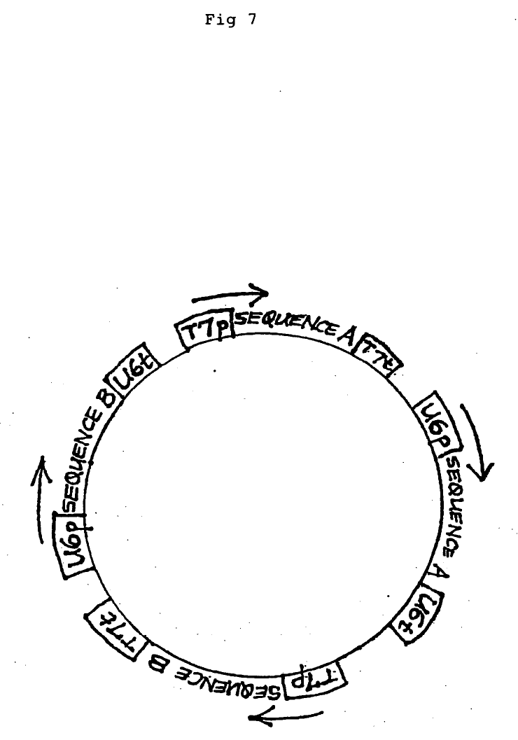

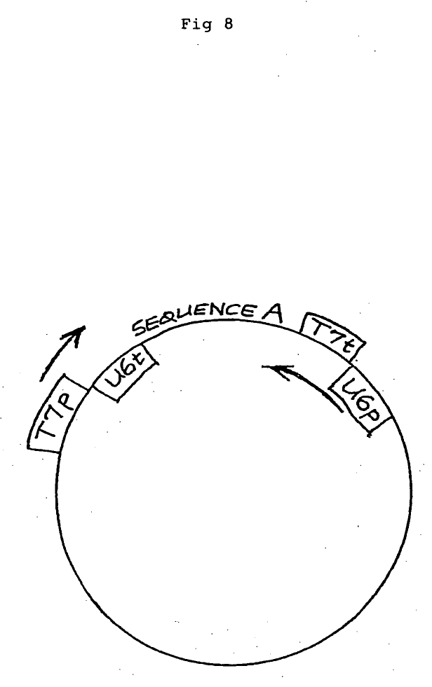

1) The multi-compartment eukaryotic expression system, comprising one or more expression constructs used in concert, must include at least two promoter elements in a single vector or set of vectors, in which each promoter is active in a different subcellular compartment of a single eukaryotic cell (meaning either different physical subcompartments or different functional domains within a single physical subcompartment). Other promoters used in said expression system may be active in the same or different subcellular compartments. For example, a vector or combination of vectors can contain two promoters such as an RNA pol III based promoter (active in the nucleolus) and an RNA pol II promoter (active in the non-nucleolus compartment of the nucleus). Promoters can be convergent or divergent with respect to each other.
2) An expression system or expression construct can contain more than one compartment specific promoter of the same type (for example, two RNA pol II promoters), if the expression system or expression construct also includes at least one promoter transcriptionally active in a different compartment.
3) Preferably, in “flanked promoter arrangements” each promoter must be different from the other promoter in the arrangement in terms of which cellular compartment the promoter is active in. For example, two T7 promoters cannot be used nor can two RNA pol II promoters. However, one T7 promoter can be used in conjunction with an RNA pol II or RNA pol III promoter. Likewise, an RNA pol III promoter can be used in conjunction with an RNA pol II promoter, or a mitochondrial promoter can be used in conjunction with a T7 promoter. This promoter arrangement is useful when transcribing a long or short hairpin RNA such as those used for post-transcriptional gene-silencing, transcriptional gene-silencing, RNAi, etc.
4) When a “flanked promoter arrangement” (or in a Class II Dual/Embedded, or a Dual/Tandem promoter system discussed below) is used, there may be a single copy of a sequence of interest, e.g. a sequence encoding a hairpin dsRNA molecule, otherwise there should be at least two copies of a sequence of interest.
5) This system can also be used to produce two different sequences of interest. For example Sequence A can be transcribed in the nucleus and Sequence B can be transcribed in the cytoplasm. For example, a vector containing the T7 RNA polymerase gene (Sequence A) can be under the transcriptional control of an RNA pol II promoter such as the HCMV intermediate early promoter and a sequence encoding a hairpin RNA (Sequence B) can be under the control of the T7 promoter (FIG. 4). The T7 RNA polymerase mRNA will be transcribed in the nucleus and translated in the cytoplasm where it can direct transcription of Sequence B from vectors located in the cytoplasm. A second example is a vector containing the human or mouse mitochondrial RNA polymerase gene (Sequence A) under the control of an RNA pol II promoter such as the MCMV immediate early (ie) promoter and a hairpin RNA (Sequence B) under the control of the human or mouse mitochondrial promoter. There are only two known mitochondrial promoters for a species: the heavy strand promoter and the light strand promoter. Either of these promoters can be used in the constructs.
   Class II: In these expression constructs, two or more promoters are located in tandem with respect to each other. The promoters can be directly juxtaposed, or separated by spacer nucleotides. Most preferably no spacer is present, less preferably a spacer of 1-100 bp is present, least preferably a spacer of 101-500 bp is present between the two promoters. In some instances, one promoter can be embedded in the second promoter. For example, the T7 or SP6 promoter can be placed within nucleotides −1 to about −20 of an RNA pol II promoter such as, e.g., the HCMV ie, MCMV ie, simian cytomegalovirus (SCMV) ie, or RSV promoter, to name a few. See FIG. 4, which illustrates embedded HCMV and T7 promoters located in tandem at the 5′ end of the T7 RNA polymerase gene. (Terminator and polyadenylation site are not shown.) The promoters in this arrangement must be different with respect to each other in terms of which cellular compartment they are active in. In this arrangement a single sequence is flanked by two or more promoters (each/all of which are located at the 5′ end of the sequence of interest). FIG. 5 illustrates three promoters, P1, P2, and P3, arranged in tandem at the 5′ end of the sequence of interest. They differ from each other in terms of the cellular compartment in which they are transcriptionally active. In another example, an RNA pol II promoter such as HCMV could be followed by the T7 promoter. In the cytoplasm of a cell expressing T7 RNA polymerase, the sequence of interest will be transcribed by T7 RNA polymerase. The vectors that localize to the non-nucleolus compartment of the nucleus can be transcribed by RNA pol II. Such an arrangement is seen in, e.g., FIG. 6, which utilizes the RNA pol II promoter HCMV and the cytoplasmic T7 promoter in tandem to drive the sequence of interest. Nuclear transcripts from the HCMV promoter will include a 5′ cap, a T7 promoter sequence, and the sequence of interest. Cytoplasmic transcripts from the T7 promoter will include only the sequence of interest. There can be one or more of these arrangements per vector.

Desirably, a promoter is operably linked to a nucleic acid sequence, for example, a cDNA or a gene sequence, or a therapeutic RNA coding sequence, in such a way as to enable expression of the nucleic acid sequence. In one aspect a promoter is operably linked to a sequence encoding a therapeutic RNA, desirably a double-stranded RNA. In one aspect, an expression construct of the invention will comprise at least two different RNA polymerase promoters selected from an RNA polymerase I promoter, an RNA polymerase II promoter, an RNA polymerase III promoter, a mitochondrial polymerase promoter, and, optionally, a bacteriophage promoter, each of said two different promoters operably linked to a sequence encoding a therapeutic RNA molecule. In one aspect the therapeutic RNA molecules are dsRNA molecules, which may be the same or different.

Promoters for this invention can be ones recognized by endogenous RNA polymerases such as RNA pol I, II, or III. They can also be ones recognized by exogenously added RNA polymerases such as viral and bacteriophage RNA polymerases. While it may be convenient to provide or express an exogenous RNA polymerase (e.g., by transfecting the host cell with an expression vector encoding the necessary polymerase) such as a viral or bacteriophage polymerase (e.g., T7, SP3, or SP6 RNA polymerase) in eukaryotic cells in cell culture or in vitro, this may be less desirable in cells in a eukaryotic organism in vivo, particularly in a vertebrate organism, where a foreign protein may induce a potentially undesirable immune response. Accordingly, in therapeutic or other applications in eukaryotic cells in vivo in some preferred embodiments methods and expression constructs will utilize only promoters active with polymerases endogenous to the cell in which transcription is desired (e.g., cellular RNA polymerase I, RNA polymerase II RNA polymerase III, and mitochondrial polymerase). Applications include transcription of RNAs such as mRNAs, ribozymes, hairpin RNAs, structured RNAs and other functional RNAs such as those involved in gene-silencing. Vectors can include, e.g., DNA plasmids and episomes and can be viral vectors. In some aspects, the expression constructs of the invention will transcribe under the control of at least two different RNA polymerase promoters, i.e., at least two therapeutic RNA molecules which regulate or decrease expression of a target gene in the target cell. The therapeutic RNA molecules are not translated into a polypeptide or protein, but rather are themselves biologically active RNAs. The therapeutic RNA molecules may be the same or different. In one aspect, the therapeutic RNA molecules include without limitation dsRNAs, shRNAs, siRNAs, antisense RNAs, combinations of a sense and an antisense RNA transcript designed to form a duplex dsRNA, ribozyme RNAs, triplex-forming RNAs, artificially selected high affinity RNA ligands (aptamer), short hairpin double-stranded RNAs, microRNAs, etc., which are active per se and not as a protein or polypeptide. The multiple therapeutic RNA molecules may be the same or different, including different transcripts which are processed to the same therapeutic RNA molecule.

Promoter Classification

RNA pol I and RNA pol III are both nucleolar promoters but RNA pol I and pol III are transcribed in distinct functional domains with respect to each other, and so included within the claimed invention is the case where an RNA pol I promoter is used with an RNA pol III promoter.

In certain embodiments of the present invention, polymerase III promoters are used, including the U6 promoter. It will be appreciated, however, that not only the U6 promoter per se, but other members of the class of RNA Polymerase III, Type 2 and Type 3 promoters, i.e., “U6-type” promoters (of which the U6 promoter in one of the U6 genes is a well-characterized member) may be used in place of the U6 promoter. Examples of other “U6-Type” promoters include H1, 7SK, and MRP as reviewed by Schramm & Hernandez (Genes Dev. 16:2593-2620 (2002)) and Paule & White (Nucleic Acids Res. 28:1283-98 (2000)). See also U.S. Pat. No. 5,624,803, Noonberg et al., with respect to U6-type polymerase III promoters, the teaching of which is hereby incorporated by reference.

RNA pol II promoters are transcribed in the non-nucleolar compartments of the nucleus. However, these promoters can be sub-divided as above with respect to nuclear domains/nuclear functional sub-compartments. Specific examples are provided.

Cytoplasmic promoters for DNA-dependent RNA polymerases are the bacteriophage promoters for example, including T7, SP6 and SP3. Promoters that are transcribed by RNA-dependent RNA polymerases would be included on RNA molecules and include many RNA viral promoters such as those derived from alpha viruses, flaviviruses, toga viruses, coronaviruses, and rhabdoviruses for example.

Mitochondrial promoters include the heavy chain and the light chain promoters.

Definitions: In the present disclosure, the following terms below are used according to the customary understanding of those skilled in this art, as more particularly defined herein.

By “compartment(s)”, “subcellular compartment(s)” or “cellular compartment(s)” of a eukaryotic cell is meant, e.g, a subcellular location within a eukaryotic cell defined primarily by a membrane and the ability to carry out specialized function(s) as well as having specific multi-protein complexes associated with transcriptional activities, e.g., the cytoplasm, the mitochondrion, the nucleus (non-nucleolus), and the nucleolus. A “compartment” or “subcellular compartment” or “cellular compartment” can also be a “functional compartment” or “functional domain” which is not physically separated from other compartments by a cell membrane, but which is defined biochemically by the presence of specific protein complexes that carry out particular functions, such as transcription from specified promoters, for example (Yamagoe S. et al., Mol. Cell. Biol. 23:1025-1033 (2003)). For purposes of the invention, RNA polymerase I, RNA polymerase II, RNA polymerase III, mitochondrial polymerase, and cytoplasmic polymerases (e.g., T7) are each considered to be transcriptionally active in a different and distinct subcellular compartment/functional domain.

Nuclear domains which are functional subcellular compartments within a cell have been identified and specific functions have been associated with them (Ascoli. C. A. and Maul G. G., J. Cell Biol. 112:785-795 (1991); Pombo et al., EMBO J. 18: 2241 (1999)). For the purposes of this invention, the only functional domains that are relevant are those that are associated with transcription of specific promoters or specific classes of promoters. For example, transcription of the herpes virus 1 genome by RNA pol II has been shown to occur within a specific nuclear domain known as ND10 (Tang et al., J. Virol. 77:5821-5828 (2003)). These herpes viral promoters are transcribed by RNA pol II in a specific sub-nuclear domain called ND10. Therefore, two RNA pol II promoters can be used together in the chemical multi-compartment promoter system (in the absence of any other promoter) only if the promoters are transcribed in functionally distinct domains. For example, a Herpes promoter can be used in conjunction with a non-herpes promoter that is also transcribed by RNA pol II, e.g., actin promoter.

By “transcription” is meant the enzymatic process whereby the genetic information contained in one strand of DNA is used to specify a complementary sequence of bases in an RNA chain, e.g, an mRNA, an antisense RNA, a dsRNA-forming RNA, a ribozyme.

By “transcription unit” or “cistron” is meant a unit in which transcription occurs. Usually a “transcription unit” means a promoter sequence operably linked to a nucleic acid sequence to be transcribed, optionally with a terminator or polyadenylation signal. Two promoters operably linked to initiate transcription of a single nucleic acid sequence, e.g., two promoters flanking a nucleic acid sequence constitutes two transcription units or two cistrons; three promoters operably linked to a single nucleic acid sequence to be transcribed constitutes three transcription units, etc. An expression construct or expression vector is commonly engineered to contain multiple transcription units or cistrons, e.g., see FIG. 7, which illustrates a plasmid with four separate cistrons.

By “transcriptionally active” is meant that a promoter sequence is capable of initiating transcription under appropriate circumstances, including, e.g., the provision of the requisite non-endogenous RNA polymerase for promoters such as bacteriophage promoters, e.g. T7, SP6, T3, or under other appropriate conditions or signals, as e.g., with inducible promoters. In one aspect of the invention, methods and constructs will utilize at least two promoters that are transcriptionally active in conjunction with endogenous polymerases of the host cell, e.g., mammalian cellular RNA polymerase I, mammalian RNA polymerase II, RNA polymerase III, and mitochondrial polymerase.

By “expression system” is meant one or more expression constructs or vectors used in concert to provide a single eukaryotic cell with at least two transcription units which are active in different subcellular compartments of the cell. The expression system may consist of a single expression construct comprising two or more transcription units or it may comprise two, three, four or more expression constructs used in concert. In some aspects the two or more expression constructs will be used as a single composition, as e.g., a pharmaceutical composition, or a research reagent. In some aspects the two or more expression constructs will be used in two or more compositions used in concert, as for example, administered simultaneously or within a period of minutes, hours or days of each other, e.g., within one, two, three days, or even a week of each other; so long as there will be some functional interaction between the products of each construct delivered to the eukaryotic cell. The expression construct(s) collectively comprise two or more promoters, including at least two promoters each of which is transcriptionally active within a different subcellular compartment of a single eukaryotic cell. The expression system serves to provide a single eukaryotic cell with one or more expression constructs comprising two or more transcription units, active in at least two subcellular compartments of the same eukaryotic cell. For purposes of this application, a “transcription unit” means a promoter sequence operably linked to a nucleic acid sequence to be transcribed, optionally with a terminator or polyadenylation signal. Two promoters operably linked to a single nucleic acid sequence constitutes two transcription units, three promoters operably linked to a single transcription unit constitutes three transcription units, etc. In one aspect of the invention, a single eukaryotic cell is provided with two or more expression constructs, which will be present in and transcriptionally active in at least two subcellular compartments of a single eukaryotic cell at the same time, or at substantially the same time. In another aspect, two or more different expression constructs or a single expression construct containing two or more transcription units will be present in a single eukaryotic cell at the same time, or at substantially the same time, but the transcription units may not be transcriptionally active at the same time, as, e.g., when it is desired to regulate transcription from one or more of the promoters, as, e.g., with inducible promoters, so that the timing of transcription from one or more of the transcription units is regulated. In one aspect the expression system will be a single expression construct or two or more expression constructs, e.g., a plasmid or plasmids, comprising two or more promoters, e.g., two, three, four, five, six, seven, eight, or more, at least two of which are transcriptionally active in different subcellular compartments of the same eukaryotic cell. In desirable embodiments, the expression system will comprise promoters transcriptionally active in two, three, four, five, or more different subcellular compartments of the same eukaryotic cell, e.g., including without limitation, cytoplasm, nucleus, mitochondria, and nucleolus, as well as functional subcellular compartments defined biochemically by the presence of specific multi-protein complexes capable of carrying out defined functions such as transcription from specific promoters, e.g., cytoplasm and nucleus, cytoplasm and nucleolus, cytoplasm and mitochondria; mitochondria and nucleus; mitochondria and nucleolus; nucleus and nucleolus; cytoplasm, nucleus, and nucleolus; cytoplasm, nucleus, and mitochondria; cytoplasm, nucleolus, and mitochondria; mitochondria, nucleolus, and nucleus; as well as cytoplasm, nucleus, mitochondria, and nucleolus. In addition, the invention contemplates using combinations of promoters transcriptionally active in different functional domains of a single subcellular compartment.

By “mitochondrion”, is meant a membrane-bound organelle found in the cytoplasm of eukaryotic cells that produces energy and contains its own DNA genome, but is dependent on the cell for proteins encoded by the nuclear genome, including the mitochondrial RNA polymerase.

By “nucleus” or “nuclear” is meant an internal compartment of a eukaryotic cell surrounded by a nuclear membrane and containing the chromosomes. For purposes of this patent application, the nucleus is considered different and distinct from the nucleolus. There are different transcriptional or functional domains within the nucleus of eukaryotic cells where different polymerases are active, e.g., different and distinct functional RNA polymerase II and RNA polymerase III domains.

By “nucleolus”, “nucleolar”, or “nucleolar non-nuclear” is meant a large, diffuse structure within the nucleus of a eukaryotic cell which contains large loops of DNA whose ribosomal RNA (rRNA) genes are transcribed at a furious rate by RNA polymerase I. Such rRNA is immediately packaged in the nucleolus with ribosomal proteins to generate the ribosomes. For purposes of this patent application, the nucleolus is considered different and distinct from the nucleus. While the nucleolus is typically associated with polymerase I transcription, more recent work indicates that in at least some eukaryotic cells RNA polymerase III genes such as 5S rRNA and various tRNAs are also transcribed in the nucleolus. See Thompson et al., “Nucleolar Clustering of Dispersed tRNA Genes”, Science 302:1399-1401 (2003). The nucleus itself is also considered to include different transcriptionally active functional domains or subcompartments in that polymerase II and polymerase III transcription occurs within different and distinct transcriptional sites of the nucleoplasm. In fact, RNA polymerase I, RNA polymerase II, and RNA polymerase III are all concentrated within their own dedicated transcription sites or “factories” within the nucleoplasm and nucleolus. See Pombo et al., “Regional specialization in the human nuclei: visualization of discrete sites of transcription by RNA polymerase III”, EMBO 18 (8):2241-2253 (1999). Accordingly, RNA polymerases I, II, and III, as well as mitochondrial and cytoplasmic polymerases (and their cognate promoters) are each considered to function within different and distinct physical or functional subcellular compartments or domains for purposes of their use in the expression constructs and methods of the invention.

By “eukaryote” or “eukaryotic cell” is meant an advanced cell of a higher organism, plant or animal, invertebrate and vertebrate, which has several chromosomes and a nucleus.

By “prokaryote” or “prokaryotic cell” is meant a primitive type of cell of lower organisms such as bacteria which contains only a single chromosome and has no nuclear membrane.

By “RNA polymerase” is meant an enzyme that synthesizes RNA. A DNA-dependent RNA polymerase synthesizes an RNA transcript from a DNA template. A RNA-dependent RNA polymerase synthesizes an RNA transcript from an RNA template. Common examples of DNA-dependent RNA polymerases active in eukaryotic cells include RNA polymerase I (RNA pol I), RNA polymerase II (RNA pol II), RNA polymerase III (RNA pol III), mitochondrial polymerase, as well as other polymerases provided to a eukaryotic cell, e.g., a bacteriophage polymerase. Preferred in some embodiments, especially in vertebrates including mammals for in vivo applications, are expression constructs which utilize for transcription RNA polymerases endogenous to the host cell, as opposed to viral or bacteriophage RNA polymerases, which have to be supplied to the host cell, e.g., encoded in an expression vector.

“Sense” and “antisense”: By “sense” is meant a nucleic acid sequence having the same sequence and orientation as found in an mRNA. By “antisense” is meant a nucleic acid complementary and of opposite orientation to a sense nucleic acid.

By an “expression construct” is meant any double-stranded DNA or double-stranded RNA designed to transcribe an RNA, e.g., a construct that contains at least one promoter operably linked to a downstream gene or coding region of interest (e.g., a cDNA or genomic DNA fragment that encodes a protein, or any RNA of interest). Transfection or transformation of the expression construct into a recipient cell allows the cell to express RNA or protein encoded by the expression construct. An expression construct may be a genetically engineered plasmid, virus, or an artificial chromosome derived from, for example, a bacteriophage, adenovirus, retrovirus, poxvirus, or herpesvirus, or further embodiments described under “expression vector” below. An expression construct can be replicated in a living cell, or it can be made synthetically. For purposes of this application, the terms “expression construct”, “expression vector”, “vector”, and “plasmid” are used interchangeably to demonstrate the application of the invention in a general, illustrative sense, and are not intended to limit the invention to a particular type of expression construct.

By “expression vector” is meant a DNA construct that contains at least one promoter operably linked to a downstream gene or coding region (e.g., a cDNA or genomic DNA fragment that encodes a protein, optionally, operatively linked to sequence lying outside a coding region, an antisense RNA coding region, or RNA sequences lying outside a coding region). Transfection or transformation of the expression vector into a recipient cell allows the cell to express RNA encoded by the expression vector. An expression vector may be a genetically engineered plasmid, virus, or artificial chromosome derived from, for example, a bacteriophage, adenovirus, retrovirus, poxvirus, or herpesvirus. Such expression vectors can include sequences from bacteria, viruses or phages. Such vectors include chromosomal, episomal and virus-derived vectors e.g., vectors derived from bacterial plasmids, bacteriophages, yeast episomes, yeast chromosomal elements, and viruses, vectors derived from combinations thereof, such as those derived from plasmid and bacteriophage genetic elements, cosmids and phagemids. Thus, one exemplary vector is a double-stranded DNA phage vector. Another exemplary vector is a double-stranded DNA viral vector.

This double-stranded or partially double-stranded molecule may be a DNA vector, a DNA plasmid or any double-stranded DNA construct designed to deliver a polynucleotide sequence to a cell for expression in the cell. This double-stranded molecule is linear in one embodiment, in another embodiment, this double stranded molecule is circular. The DNA molecule may be a double stranded plasmid or vector containing sequences under the control of any desired combination of promoters as described elsewhere herein, including RNA pol I, and/or RNA pol II, and/or RNA pol III, and/or mitochondrial promoters; and/or viral, bacterial, and bacteriophage promoters. Desirably the vector will express one, two, three, four, five or more therapeutic RNA molecules, e.g., dsRNA molecules, antisense molecules, ribozymes, aptamers, etc. under the control of two, three, four, five or more promoters, including at least two different promoters selected from an RNA pol I promoter, an RNA pol II promoter, an RNA pol III promoter, a mitochondrial promoter, and optionally a cytoplasmic promoter. In one aspect of the invention, the expression construct will include promoters active with polymerases endogenous to the host cell. In some methods and expression constructs of the invention, a non-endogenous phage or viral promoter/polymerase system such as T7 or SP6 may be utilized in conjunction with a promoter/endogenous polymerase system to achieve cytoplasmic expression of a desired therapeutic RNA as well as expression of a therapeutic RNA in a different physical or functional subcompartment of the same eukaryotic cell. Preferably, where the promoter is an RNA pol II promoter, the sequence encoding the RNA molecule has an open reading frame greater than about 300 nucleotides to avoid degradation in the nucleus via nonsense mRNA surveillance degradation mechanisms. Such plasmids or vectors can include sequences from bacteria, viruses or phages.

The term “isolated” is meant to refer to material which is substantially or essentially free from components which normally accompany the material as found in its native state. Thus, an isolated protein does not include materials normally associated with its in situ environment. An isolated nucleic acid is substantially free from sequences within which it would normally exist in the natural state, e.g., a gene isolated from other chromosomal sequences which would normally flank it in the natural state, a promoter isolated from the gene it normally expresses, or free from other cellular materials, including proteins and other molecules. Typically, isolated proteins or nucleic acids of the invention are at least about 80% pure, usually at least about 90%, preferably at least about 95% or even greater than 99% pure as measured by accepted analytical methods for determining purity. In the present invention polypeptides are purified from transgenic cells.

A “heterologous sequence” or a “heterologous nucleic acid”, as used herein, is one that originates from a foreign source (or species) or, if from the same source, is modified from its original form. Thus, a heterologous nucleic acid operably linked to a promoter is from a source different from that from which the promoter was derived, or, if from the same source, is modified from its original form. For example, a UDPglucose 4-epimerase gene promoter can be linked to a structural gene encoding a polypeptide other than native UDPglucose 4-epimerase. Modification of the heterologous sequence may occur, e.g., by treating the DNA with a restriction enzyme to generate a DNA fragment that is capable of being operably linked to the promoter. Techniques such as site-directed mutagenesis are also useful for modifying a heterologous sequence.

The term “operably linked” refers to functional linkage between a nucleic acid expression control sequence (such as a promoter, signal sequence, enhancer or array of transcription factor binding sites) and a second nucleic acid sequence, wherein the expression control sequence affects transcription and/or translation of the nucleic acid corresponding to the second sequence when the appropriate molecules (e.g., transcriptional activator proteins) are bound to the expression control sequence.

The term “recombinant” when used with reference to a cell indicates that the cell replicates a heterologous nucleic acid, or expresses a peptide or protein encoded by a heterologous nucleic acid. Recombinant cells can express genes that are not found within the native (non-recombinant) form of the cell. Recombinant cells can also express genes that are found in the native form of the cell, but wherein the genes are modified and re-introduced into the cell by artificial means.

By “agent that provides an at least partially double-stranded RNA” is meant a composition that generates an at least partially double-stranded (ds)RNA in a cell or animal. For example, the agent can be a dsRNA, a single stranded RNA molecule that assumes a double stranded conformation inside the cell or animal (e.g., a hairpin), or a combination of two single stranded RNA molecules that are administered simultaneously or sequentially and that assume a double stranded conformation inside the cell or animal. Other exemplary agents include a DNA molecule, plasmid, viral vector, or recombinant virus encoding an at least partially dsRNA. Other agents are disclosed in WO 00/63364, filed Apr. 19, 2000. In some embodiments, the agent includes between 1 ng and 20 mg, 1 ng to 1 μg, 1 μg to 1 mg, or 1 mg to 20 mg of DNA and/or RNA. Expression constructs of the invention may encode two, three, four, five or more dsRNA molecules, which may be the same or different. Said expression constructs will include promoters from at least two of RNA pol I promoters, RNA pol II promoters, RNA pol III promoters, cytoplasmic promoters, and mitochondrial promoters operably linked to a sequence encoding a therapeutic RNA molecule, e.g., an at least partially dsRNA molecule.

By “antisense” or “antisense applications” or “antisense technology” or “antisense therapeutics” is meant an approach to inhibiting gene expression, including oncogene expression, viral gene expression, etc. An “antisense” RNA molecule is one which contains the complement of, and can therefore hybridize with, protein-encoding RNAs present in the target cell. It is believed that the hybridization of antisense RNA to its cellular RNA complement can prevent expression of the cellular RNA, perhaps by limiting its translatability. While various studies have involved the processing of RNA or direct introduction of antisense RNA oligonucleotides to cells for the inhibition of gene expression (Brown et al., Oncogene Res. 4:243-52 (1989); Wickstrom et al., Proc. Natl. Acad. Sci. USA 85:1028-32 (1988); Smith et al., 1986; Buvoli et al., Nucleic Acids Res. 15:9091 (1987)), the more promising means of cellular introduction of antisense RNAs has been through the construction of recombinant vectors which will express antisense RNA once the vector is introduced into the cell. Accordingly, the multi-compartment expression systems of the invention are highly relevant to more efficient expression of antisense molecules in eukaryotic cells, especially for in vivo applications.

A principal application of antisense RNA technology has been in connection with attempts to affect the expression of specific genes. For example, Delauney et al. have reported the use of antisense transcripts to inhibit gene expression in transgenic plants (Delauney et al., Proc. Natl. Acad. Sci. USA 85:4300-04 (1988)). These authors report the down-regulation of chloramphenicol acetyl transferase activity in tobacco plants transformed with CAT sequences through the application of antisense technology.

Antisense technology has also been applied in attempts to inhibit the expression of various oncogenes. For example, Kasid et al., Science 243:1354-6 (1989), report the preparation of a recombinant vector construct employing Craf-1 cDNA fragments in an antisense orientation, brought under the control of an adenovirus 2 late promoter. These authors report that the introduction of this recombinant construct into a human squamous cell carcinoma resulted in a greatly reduced tumorigenic potential relative to cells transfected with control sense transfectants. Similarly, Prochownik et al., Mol. Cell Biol. 8:3683-95 (1988), have reported the use of cmyc antisense constructs to accelerate differentiation and inhibit G₁ progression in Friend Murine Erythroleukemia cells. In contrast, Khokha et al., Science 243:947-50 (1989), discloses the use of antisense RNAs to confer oncogenicity on 3T3 cells, through the use of antisense RNA to reduce murine tissue inhibitor or metalloproteinase levels.

Antisense methodology takes advantage of the fact that nucleic acids tend to pair with “complementary” sequences. Complementary sequences are those polynucleotides which are capable of base-pairing according to the standard Watson-Crick complementarity rules. That is, the larger purines will base pair with the smaller pyrimidines to form combinations of guanine paired with cytosine (G:C) and adenine paired with either thymine (A:T) in the case of DNA, or adenine paired with uracil (A:U) in the case of RNA. Inclusion of less common bases such as inosine, 5-methylcytosine, 6-methyladenine, hypoxanthine and others in hybridizing sequences does not interfere with pairing. Targeting double-stranded DNA with polynucleotides leads to triple-helix or triplex formation; targeting RNA will lead to double-helix formation. Antisense polynucleotides, when introduced into a target cell, specifically bind to their target polynucleotide and interfere with transcription, RNA processing, transport, translation and/or stability. DNA expression vectors encoding such antisense RNAs may be employed to inhibit gene transcription or translation or both within a host cell, either in vitro or in vivo, such as within a host animal, including a human subject.

By “DNA vaccines” or “DNA immunization” or “DNA-based vaccines” or “genetic immunization” is meant the use of DNA, as e.g., an expression construct, as a means for delivery of an antigen into a host organism, including a mammalian organism such as a human, for immunization purposes. Vaccination and immunization generally refer to the introduction of a non-virulent agent against which an individual's immune system can initiate an immune response which will then be available to defend against challenge by a pathogen. The immune system identifies invading “foreign” compositions and agents primarily by identifying proteins and other large molecules which are not normally present in the individual. The foreign protein represents a target against which the immune response is made.

DNA vaccines utilize genetic material that encodes an immunogenic peptide or protein, which is directly administered to an individual either in vivo or to the cells of an individual ex vivo. The genetic material encodes a peptide or protein that shares at least an epitope with an immunogenic protein to be targeted. The genetic material is expressed by the individual's cells to form immunogenic target proteins that elicit an immune response. The resulting immune response is broad based, involving both the humo'ral and cellular arms of the immune response. Thus, the immune responses elicited by DNA-based vaccination methods are particularly effective to protect against pathogen infection, especially intracellular pathogens such as viruses, or combat cells associated with hyperproliferative diseases or autoimmune diseases.

The immune response elicited by the target protein that is produced by vaccinated cells in an individual is a broad-based immune response which involves B cell and T cell responses including cytotoxic T cell (CTL) responses. The target antigens produced within the cells of the host are processed intracellularly: broken down into small peptides, bound by Class I MHC (major histocompatibility complex) molecules, and expressed on the cell surface. The Class I MHC-target antigen complexes are capable of stimulating CD8+ T-cells, which are phenotypically the killer/suppressor cells. Genetic immunization is thus capable of eliciting CTL responses (killer cell responses). It has been observed that genetic immunization is more likely to elicit CTL responses than other methods of immunization.

Direct injection of DNA into animals is a promising method for delivering specific antigens for immunization (Barry et al., BioTechniques 16:616-619 (1994); Davis et al., Hum. Mol. Genet. 11:1847-1851 (1993); Tang et al., Nature 356:152-154 (1992); Wang et al., J. Virol. 670:3338-3344 (1993); and Wolff et al., Science 247:1465-1468 (1990)). This approach has been successfully used to generate protective immunity against influenza virus in mice and chickens, against bovine herpes virus 1 in mice and cattle, and against rabies virus in mice (Cox et al., J. Virol. 67:5664-5667 (1993); Fynan et al, DNA Cell Biol. 12:785-789 (1993); Ulmer et al., Science 259:1745-1749 (1993); and Xiang et al., Virology 199:132-140 (1994)). In most cases, strong, yet highly variable, antibody and cytotoxic T-cell responses were associated with control of infection. Indeed, the potential to generate long-lasting memory CTLs makes this approach particularly attractive compared with those involving killed-virus vaccines and generating an antibody response. However, like other in vivo applications where nucleic acid delivery tends to be relatively poor, the efficacy of DNA vaccines could be vastly improved by the methods of the invention. Accordingly, the multi-compartment expression systems of the invention are highly relevant to more efficient expression of immunogenic peptides or proteins from DNA vaccine molecules in eukaryotic cells, especially for in vivo applications.

By “alteration in the level of gene expression” is meant a change in transcription, translation, or mRNA or protein stability, such that the overall amount of a product of the gene, i.e., mRNA or polypeptide, is increased or decreased.

By “bacterial infection” is meant the invasion of a host animal by pathogenic bacteria. For example, the infection may include the excessive growth of bacteria that are normally present in or on the body of an animal or growth of bacteria that are not normally present in or on the animal. More generally, a bacterial infection can be any situation in which the presence of a bacterial population(s) is damaging to a host animal. Thus, an animal is “suffering” from a bacterial infection when an excessive amount of a bacterial population is present in or on the animal's body, or when the presence of a bacterial population(s) is damaging the cells or tissue(s) of the animal. In one embodiment, the number of a particular genus or species of bacteria is at least 2, 4, 6, or 8 times the number normally found in the animal: The bacterial infection may be due to gram positive and/or gram negative bacteria.

By “a decrease” is meant a lowering in the level of: a) protein (e.g., as measured by ELISA or Western blot analysis); b) reporter gene activity (e.g., as measured by reporter gene assay, for example, β-galactosidase, green fluorescent protein, or luciferase activity); c) mRNA (e.g., as measured by RT-PCR or Northern blot analysis relative to an internal control, such as a “housekeeping” gene product, for example, β-actin or glyceraldehyde 3-phosphate dehydrogenase (GAPDH)); or d) cell function, for example, as assayed by the number of apoptotic, mobile, growing, cell cycle arrested, invasive, differentiated, or dedifferentiated cells in a test sample. In all cases, the lowering is desirably by at least 20%, more desirably by at least 30%, 40%, 50%, 60%, 75%, and most desirably by at least 90%. As used herein, a decrease may be the direct or indirect result of PTGS, TGS, or another gene silencing event.

By “nucleic acid molecule” is meant a compound in which one or more molecules of phosphoric acid are combined with a carbohydrate (e.g., pentose or hexose) which are in turn combined with bases derived from purine (e.g., adenine or guanine) and from pyrimidine (e.g., thymine, cytosine, or uracil). Particular naturally-occurring nucleic acid molecules include genomic deoxyribonucleic acid (DNA) and genomic ribonucleic acid (RNA), as well as the several different forms of the latter, e.g., messenger RNA (mRNA), transfer RNA (tRNA), and ribosomal RNA (rRNA), as well as catalytic RNA structures such as ribozymes. Also included are different DNA molecules which are complementary (cDNA) to the different RNA molecules. Synthesized DNA, or a hybrid thereof with naturally-occurring DNA, as well as DNA/RNA hybrids, and PNA molecules (Gambari, Curr. Pharm. Des. 7:1839-62 (2001)) are also included within the definition of “nucleic acid molecule.”.

Nucleic acids typically have a sequence of two or more covalently bonded naturally-occurring or modified deoxyribonucleotides or ribonucleotides. Modified nucleic acids include, e.g., peptide nucleic acids and nucleotides with unnatural bases. Modifications include those chemical and structural modifications described under the definition of “dsRNA” below. Also included are, e.g., various structures, as described within the definitions of “dsRNA”, “expression vectors”, and “expression constructs”, and elsewhere in this specification.

By “dsRNA” is meant a nucleic acid molecule containing a region of 17 or more, preferably at least 19 or more, basepairs that are in a double stranded conformation. In various embodiments, the dsRNA consists entirely of ribonucleotides or consists of a mixture of ribonucleotides and deoxynucleotides, such as the RNA/DNA hybrids disclosed, for example, by WO 00/63364, filed Apr. 19, 2000, or U.S. Ser. No. 60/130,377, filed Apr. 21, 1999. The dsRNA may be a single molecule with regions of self-complementarity such that nucleotides in one segment of the molecule base pair with nucleotides in another segment of the molecule. In various embodiments, a dsRNA that consists of a single molecule consists entirely of ribonucleotides or includes a region of ribonucleotides that is complementary to a region of deoxyribonucleotides. Alternatively, the dsRNA may be a duplex dsRNA, i.e., comprising two different strands (i.e., a sense transcript and an antisense transcript) that have a region of complementarity to each other. The double-stranded region will include a contiguous region of at least about 17 to 19 nucleotides complementarity to a target nucleic acid, e.g., an mRNA or a gene sequence to be down-regulated. In various embodiments, both strands consist entirely of ribonucleotides, one strand consists entirely of ribonucleotides and one strand consists entirely of deoxyribonucleotides, or one or both strands contain a mixture of ribonucleotides and deoxyribonucleotides. Desirably, the regions of complementarity are at least 70, 80, 90, 95, 98, or 100% complementary to a target nucleic acid. Desirably, the region of the dsRNA that is present in a double stranded conformation includes at least 19, 20, 30, 50, 75, 100, 200, 500, 1000, 2000, or 5000 nucleotides, or includes all of the nucleotides in a cDNA being represented in the dsRNA. In some embodiments, the dsRNA does not contain any single stranded regions, such as single stranded ends, or the dsRNA is a hairpin. In other embodiments, the dsRNA has one or more single stranded regions or overhangs. Desirable RNA/DNA hybrids include a DNA strand or region that is an antisense strand or region (e.g., has at least 70, 80, 90, 95, 98, or 100% complementarity to a target nucleic acid) and an RNA strand or region that is a sense strand or region (e.g., has at least 70, 80, 90, 95, 98, or 100% identity to a target nucleic acid), or vice versa. In various embodiments, the RNA/DNA hybrid is made in vitro using enzymatic or chemical synthetic methods such as those described herein, or those described in WO 00/63364, filed Apr. 19, 2000, or U.S. Ser. No. 60/130,377, filed Apr. 21, 1999. In other embodiments, a DNA strand synthesized in vitro is complexed with an RNA strand made in vivo or in vitro before, after, or concurrent with the transformation of the DNA strand into the cell. In yet other embodiments, the dsRNA is a single circular nucleic acid containing a sense and an antisense region, or the dsRNA includes a circular nucleic acid and either a second circular nucleic acid or a linear nucleic acid (see, for example, WO 00/63364, filed Apr. 19, 2000, or U.S. Ser. No. 60/130,377, filed Apr. 21, 1999). Exemplary circular nucleic acids include lariat structures in which the free 5′ phosphoryl group of a nucleotide becomes linked to the 2′ hydroxyl group of another nucleotide in a loop back fashion. Desirable dsRNAs include the “forced hairpins” and “partial hairpins” as taught in U.S. Provisional Application 60/399,998, “Use of Double-Stranded RNA for Identifying Nucleic Acid Sequences that Modulate the Function of a Cell”, filed Jul. 31, 2002, and PCT/US03/24028, “Double Stranded RNA Structures and Constructs and Methods for Generating and Using the Same”, filed Jul. 31, 2003, incorporated herein by reference. Other desirable hairpin dsRNAs are long dsRNAs that can be cleaved into siRNAs (short interfering dsRNAs of 19-17 base pairs) independent of Dicer or other similar enzymes, see U.S. Provisional Application 60/419,532, filed 18 Oct. 2002, and PCT/US2003/33466 filed 20 Oct. 2003 incorporated herein by reference.

In other embodiments, the dsRNA includes one or more modified nucleotides in which the 2′ position in the sugar contains a halogen (such as a fluorine group) or contains an alkoxy group (such as a methoxy group) which increases the half-life of the dsRNA in vitro or in vivo compared to the corresponding dsRNA in which the corresponding 2′ position contains a hydrogen or an hydroxyl group. In yet other embodiments, the dsRNA includes one or more linkages between adjacent nucleotides other than a naturally-occurring phosphodiester linkage. Examples of such linkages include phosphoramide, phosphorothioate, and phosphorodithioate linkages. In other embodiments, the dsRNA contains one or two capped strands or no capped strands, as disclosed, for example, by WO 00/63364, filed Apr. 19, 2000, or U.S. Ser. No. 60/130,377, filed Apr. 21, 1999. In other embodiments, the dsRNA contains coding sequence or non-coding sequence, for example, a regulatory sequence (e.g., a transcription factor binding site, a promoter, or a 5′ or 3′ untranslated region (UTR) of an mRNA). Additionally, the dsRNA can be any of the at least partially double-stranded RNA molecules disclosed in WO 00/63364, filed Apr. 19, 2000 (see, for example, pages 8-22). Any of the dsRNA molecules may be expressed in vitro or in vivo using the methods described herein, or using standard methods, such as those described in WO 00/63364, filed Apr. 19, 2000 (see, for example, pages 16-22).

By “dsRNA expression library” is meant a collection of nucleic acid expression vectors containing nucleic acid sequences, for example, cDNA sequences or randomized nucleic acid sequences that are capable of forming a dsRNA upon expression of the nucleic acid sequence. Desirably the dsRNA expression library contains at least 10,000 unique nucleic acid sequences, more desirably at least 50,000; 100,000; or 500,000 unique nucleic acid sequences, and most desirably, at least 1,000,000 unique nucleic acid sequences. By a “unique nucleic acid sequence” is meant that a nucleic acid sequence of a dsRNA expression library has desirably less than 50%, more desirably less than 25% or 20%, and most desirably less than 10% nucleic acid identity to another nucleic acid sequence of a dsRNA expression library when the full length sequence is compared. Sequence identity is typically measured using BLAST® (Basic Local Alignment Search Tool) or BLAST®2 with the default parameters specified therein (see, Altschul et al., J. Mol. Biol. 215:403-410 (1990); and Tatiana et al., FEMS Microbiol. Lett. 174:247-250 (1999)). This software program matches similar sequences by assigning degrees of homology to various substitutions, deletions, and other modifications. Conservative substitutions typically include substitutions within the following groups: glycine, alanine, valine, isoleucine, leucine; aspartic acid, glutamic acid, asparagine, glutamine; serine, threonine; lysine, arginine; and phenylalanine, tyrosine.

The preparation of cDNAs for the generation of dsRNA expression libraries is described, e.g., in U.S. Published Application 2002/0132257 and European Published Application 1229134, “Use of post-transcriptional gene silencing for identifying nucleic acid sequences that modulate the function of a cell”, the teaching of which is hereby incorporated by reference. A randomized nucleic acid library may also be generated as described, e.g., in U.S. Pat. No. 5,639,595, the teaching of which is hereby incorporated by reference, and utilized for dsRNA-mediated functional genomics applications. The dsRNA expression library may contain nucleic acid sequences that are transcribed in the nucleus or that are transcribed in the cytoplasm of the cell. A dsRNA expression library may be generated using techniques described herein, e.g., dsRNA expression constructs which transcribe a dsRNA from at least two promoters selected from at least two different categories of RNA pol I, RNA pol II, RNA pol III, mitochondrial, and cytoplasmic promoters.

By “forced hairpin” is meant a nucleic acid molecule (e.g., a DNA molecule or vector) or a population of nucleic acid molecules encoding an RNA (e.g., a partial or full hairpin) that has, in 5′ to 3′ order, a first region of interest, a first base-paired region, a loop region, and a second base-paired region. The first and second base-paired regions are base-paired to each other. Desirably, the nucleic acid further includes a second region of interest downstream of the second base-paired region. If the second region of interest is present, the first and second regions of interest are base-paired to each other. Desirably, at least 50, 60, 70, 80, 90, 95, or 100% of the nucleotides in first and second regions of interest participate in Watson-Crick base-pairing with each other. These two regions may be the same length or may differ in length by one or more nucleotides. For example, one region of interest may have additional nucleotides at one end of the region that are not base-paired to nucleotides in any portion of the other region of interest. In a related aspect, the invention features an RNA molecule or a population of RNA molecules encoded by these nucleic acids. See the teaching of U.S. Provisional Application 60/399,998, “Use of Double-Stranded RNA for Identifying Nucleic Acid Sequences that Modulate the Function of a Cell”, filed Jul. 31, 2002, and PCT/US03/24028, “Double Stranded RNA Structures and Constructs and Methods for Generating and Using the Same”, filed Jul. 31, 2003, incorporated herein by reference.

By “full RNA hairpin” is meant a hairpin without a single stranded overhang.

By “function of a cell” is meant any cell activity that can be measured or assessed. Examples of cell function include, but are not limited to, cell motility, apoptosis, cell growth, cell invasion, vascularization, cell cycle events, cell differentiation, cell dedifferentiation, neuronal cell regeneration, and the ability of a cell to support viral replication. The function of a cell may also be to affect the function, gene expression, or the polypeptide biological activity of another cell, for example, a neighboring cell, a cell that is contacted with the cell, or a cell that is contacted with media or other extracellular fluid in which the cell is contained.

By “high stringency conditions” is meant hybridization in 2×SSC at 40° C. with a DNA probe length of at least 40 nucleotides. For other definitions of high stringency conditions, see F. Ausubel et al., Current Protocols in Molecular Biology, pp. 6.3.1-6.3.6, John Wiley & Sons, New York, N.Y., 1994, hereby incorporated by reference.

By “isolated nucleic acid,” “isolated nucleic acid sequence,” “isolated nucleic acid molecule,” “isolated dsRNA nucleic acid sequence,” or “isolated dsRNA nucleic acid” is meant a nucleic acid molecule, or a portion thereof, that is free of the genes that, in the naturally-occurring genome of the organism from which the nucleic acid sequence of the invention is derived, flank the gene. The term therefore includes, for example, a recombinant DNA, with or without 5′ or 3′ flanking sequences that is incorporated into a vector, for example, dsRNA expression vector; into an autonomously replicating plasmid or virus; or into the genomic DNA of a prokaryote or eukaryote; or which exists as a separate molecule (e.g., a cDNA or a genomic or cDNA fragment produced by PCR or restriction endonuclease digestion) independent of other sequences.

By “an increase” is meant a rise in the level of: (a) protein (e.g., as measured by ELISA or Western blot analysis); (b) reporter gene activity (e.g., as measured by reporter gene assay, for example, β-galactosidase, green fluorescent protein, or luciferase activity); (c) mRNA (e.g., as measured by RT-PCR or Northern blot analysis relative to an internal control, such as a “housekeeping” gene product, for example, β-actin or glyceraldehyde 3-phosphate dehydrogenase (GAPDH)); or (d) cell function, for example, as assayed by the number of apoptotic, mobile, growing, cell cycle arrested, invasive, differentiated, or dedifferentiated cells in a test sample. Desirably, the increase is by at least 1.5-fold to 2-fold, more desirably by at least 3-fold, and most desirably by at least 5-fold. As used herein, an increase may be the indirect result of PTGS, TGS, or another gene silencing event. For example, the dsRNA may inhibit the expression of a protein, such as a suppressor protein, that would otherwise inhibit the expression of another nucleic acid molecule.

By “long dsRNA” is meant a dsRNA that is at least 40, 50, 100, 200, 500, 1000, 2000, 5000, 10000, or more nucleotides in length. In some embodiments, the long dsRNA has a double stranded region of between 30 to 100,100 to 10000, 100 to 1000, 200 to 1000, or 200 to 500 contiguous nucleotides, inclusive. In some embodiments, the long dsRNA is a single strand which achieves a double-stranded structure by virtue of regions of self-complementarity (e.g., inverted repeats or tandem sense and antisense sequences) that result in the formation of a hairpin structure. In one embodiment, the long dsRNA molecule does not produce a functional protein or is not translated. For example, the long dsRNA may be designed not to interact with cellular factors involved in translation. Exemplary long dsRNA molecules lack a poly-adenylation sequence, a Kozak region necessary for protein translation, an initiating methionine codon, and/or a cap structure. In other embodiments, the dsRNA molecule has a cap structure, one or more introns, and/or a polyadenylation sequence. Other such long dsRNA molecules include RNA/DNA hybrids. Other dsRNA molecules that may be used in the methods of the invention and various means for their preparation and delivery are described in WO 00/63364, filed Apr. 19, 2000, the teaching of which is incorporated herein by reference.

By “modulates” is meant changing, either by a decrease or an increase. As used herein, desirably a nucleic acid molecule decreases the function of a cell, the expression of a target nucleic acid molecule in a cell, or the biological activity of a target polypeptide in a cell by least 20%, more desirably by at least 30%, 40%, 50%, 60% or 75%, and most desirably by at least 90%. Also as used herein, desirably a nucleic acid molecule increases the function of a cell, the expression of a target nucleic acid molecule in a cell, or the biological activity of a target polypeptide in a cell by at least 1.5-fold to 2-fold, more desirably by at least 3-fold, and most desirably by at least 5-fold.

By “multiple cloning site” is meant a known sequence within a DNA plasmid construct that contains a single specific restriction enzyme recognition site for one or more restriction enzymes, and that serves as the insertion site for a nucleic acid sequence. A multiple cloning site is also referred to as a polylinker or polycloning site. A wide variety of these sites are known in the art.

By “multiple epitope dsRNA” is meant an RNA molecule that has segments derived from multiple target nucleic acids or that has non-contiguous segments from the same target nucleic acid. For example, the multiple epitope dsRNA may have segments derived from (i) sequences representing multiple genes of a single organism; (ii) sequences representing one or more genes from a variety of different organisms; and/or (iii) sequences representing different regions of a particular gene (e.g., one or more sequences from a promoter and one or more sequences from a coding region such as an exon). Desirably, each segment has substantial sequence identity to the corresponding region of a target nucleic acid. In various desirable embodiments, a segment with substantial sequence identity to the target nucleic acid is at least 30, 40, 50, 100, 200, 500, 750, or more nucleotides in length. In desirable embodiments, the multiple epitope dsRNA inhibits the expression of at least 2, 4, 6, 8, 10, 15, 20, or more target genes by at least 20, 40, 60, 80, 90, 95, or 100%. In some embodiments, the multiple epitope dsRNA has non-contiguous segments from the same target gene that may or may not be in the naturally occurring 5′ to 3′ order of the segments, and the dsRNA inhibits the expression of the nucleic acid by at least 50, 100, 200, 500, or 1000% more than a dsRNA with only one of the segments.

By “partial RNA hairpin” is meant a hairpin that has a single stranded overhang, such as a 5′ or 3′ overhang. Desirably the partial hairpin will be encoded by a nucleic acid (e.g., a DNA molecule or vector). The encoded RNA molecule has, in 5′ to 3′ order, a first region of interest (Region 1), a loop region, and a second region of interest (Region 2). The regions of interest differ in length, and Region 1 has additional nucleotides at one end of the region that are not base-paired to nucleotides in the other region of interest (Region 2). One region of interest comprises a sequence of substantial identity to a target gene, and the other region of interest comprises a sequence of substantial complementarity to the target gene. In addition, the “partial” hairpin RNA may be a “forced” hairpin RNA, in which case the Region 1, which includes either a sense or antisense sequence with respect to the target gene, will also include a Sequence A, and Region 2 will include a Sequence B, designed to base-pair with at least a portion of Sequence A, which serves to “force” the RNA to assume a hairpin structure. Optionally, Region 2 will include additional 3′ nucleotides complementary to nucleotides of Region 1. In a related aspect, the invention features an RNA molecule or a population of RNA molecules encoded by these nucleic acids. Desirably, the encoded RNA inhibits expression of the target gene in a cell or animal. Desirably, the partial RNA hairpin is extended in vitro or in vivo (e.g., in a cell or animal) with an RNA dependent-RNA polymerase. Desirably, extension of the partial hairpin produces a full hairpin. See the teaching of U.S Provisional Application 60/399,998, “Use of Double-Stranded RNA for Identifying Nucleic Acid Sequences that Modulate the Function of a Cell”, filed Jul. 31, 2002, and PCT/US03/24028 “Double Stranded RNA Structures and Constructs and Methods for Generating and Using the Same”, filed Jul. 31, 2003, incorporated herein by reference.

By “phenotype” is meant, for example, any detectable or observable outward physical manifestation, such as molecules, macromolecules, structures, metabolism, energy utilization, tissues, organs, reflexes, and behaviors, as well as anything that is part of the detectable structure, function, or behavior of a cell, tissue, or living organism. Particularly useful in the methods of the invention are dsRNA mediated changes, wherein the detectable phenotype derives from modulation of the function of a cell, modulation of expression of a target nucleic acid, or modulation of the biological activity of a target polypeptide through dsRNA effects on a target nucleic acid molecule.

By “polypeptide biological activity” is meant the ability of a target polypeptide to modulate cell function. The level of polypeptide biological activity may be directly measured using standard assays known in the art. For example, the relative level of polypeptide biological activity may be assessed by measuring the level of the mRNA that encodes the target polypeptide (e.g., by reverse transcription-polymerase chain reaction (RT-PCR) amplification or Northern blot analysis); the level of target polypeptide (e.g., by ELISA or Western blot analysis); the activity of a reporter gene under the transcriptional regulation of a target polypeptide transcriptional regulatory region (e.g., by reporter gene assay, as described below); the specific interaction of a target polypeptide with another molecule, for example, a polypeptide that is activated by the target polypeptide or that inhibits the target polypeptide activity (e.g., by the two-hybrid assay); or the phosphorylation or glycosylation state of the target polypeptide. A compound, such as a dsRNA, that increases the level of the target polypeptide, mRNA encoding the target polypeptide, or reporter gene activity within a cell, a cell extract, or other experimental sample, is a compound that stimulates or increases the biological activity of a target polypeptide. A compound, such as a dsRNA, that decreases the level of the target polypeptide, mRNA encoding the target polypeptide, or reporter gene activity within a cell, a cell extract, or other experimental sample, is a compound that decreases the biological activity of a target polypeptide.

By “promoter” is meant a minimal sequence sufficient to direct transcription of a gene, including pol I promoters, pol II promoters, pol III promoters, mitochondrial promoters, and viral, bacterial, bacteriophage, and other promoter sequences that are capable of driving transcription. Also included in this definition are those transcription control elements (e.g., enhancers) that are sufficient to render promoter-dependent gene expression controllable in a cell type-specific, tissue-specific, or temporal-specific manner, or that are inducible by external signals or agents; such elements, which are well-known to skilled artisans, may be found in a 5′ or 3′ region of a gene or within an intron.

Desirably a promoter is operably linked to a nucleic acid sequence, for example, a cDNA or a gene, or a nucleic acid sequence encoding a biologically active RNA, in such a way as to permit expression of the nucleic acid sequence, e.g., as an mRNA or a therapeutic RNA, e.g., dsRNA (including duplex dsRNAs and single stranded hairpin-forming dsRNAs), ribozyme, antisense RNA, triplex-forming RNA, and, optionally, an mRNA capable of translation into a polypeptide product. In one aspect, an expression construct of the invention will include promoters operably linked to a sequence encoding a therapeutic RNA, desirably a double-stranded RNA.

For purposes of the instant invention, promoters are classified according to the specific subcellular compartment of a eukaryotic cell in which the promoter is transcriptionally active, e.g., the cytoplasm (cytoplasmic promoters), mitochondria (mitochondrial promoters), nucleus (nuclear promoters), and non-nuclear nucleolar (nucleolar promoters), as well as functionally distinct compartments such as HDAC (histone deacetylase complex). Promoters are “subcompartment-specific” or “compartment-specific” because they are transcriptionally active in distinct subcellular compartments or functional subcompartments of a eukaryotic cell. For example, RNA pol II promoters (initiate transcription by RNA pol II) are nuclear (non-nucleolar) promoters; RNA pol III promoters (initiate transcription by RNA pol I) are nucleolar promoters; RNA pol I promoters (initiate transcription by RNA pol I) are nucleolar promoters; SP6, T7, T3 and other bacteriophage promoters are active in the cytoplasm in the presence of their respective RNA polymerases (SP6, T7, T3 RNA polymerase); mitochondrial promoters, e.g., the human light chain promoter, the human heavy chain promoter, as well as animal promoters, such as murine, guinea pig, rabbit, and various primate promoters, which are transcriptionally active in mitochondria (See the teaching of WO 02/068629 published Sep. 6, 2002, Satishchandran et al.). While certain categories of promoters are believed to be active within the same physical subcompartment of a eukaryotic cell (e.g., polymerase II and polymerase III promoters within the nucleus), each of polymerase I, RNA polymerase II, and RNA polymerase III promoters (as well as cytoplasmic and mitochondrial promoters) are considered to be transcriptionally active within either a distinct physical or functional subcellular compartment or domain of a eukaryotic cell. See Pombo et al., EMBO 18 (8):2241-2253 (1999). It will be recognized that for purposes of the instant invention suitable promoters will include not only known, naturally occurring promoters but synthetic and semi-synthetic promoters designed to initiate transcription in a selected subcellular compartment. For example, see the structural promoters, including forced-open padlock promoters, and the chimeric promoters of U.S. Ser. No. 60/464,434, filed Apr. 22, 2003, and PCT US02/33669, filed Apr. 22, 2002, “Transfection Kinetics and Structural Promoters”, Pachuk and Satishchandran.

One or more of the promoters may be an inducible promoter, such as a lac (Cronin et al., Genes Dev. 15:1506-1517 (2001)), ara (Khlebnikov et al., J. Bacteriol. 182:7029-34 (2000)), ecdysone (Rheogene, www.rheogene.com), RU48 (mefepristone) (corticosteroid antagonist) (Wang et al., Proc. Natl. Acad. Sci. USA 96:8483-88 (1999)), or tet promoter (Rendal et al., Hum. Gene Ther. 13:335-42 (2002); Larnartina et al., Hum. Gene Ther. 13:199-210 (2002)) or a promoter disclosed in WO 00/63364, filed Apr. 19, 2000. When an inducible promoter is employed, it is considered “transcriptionally active” in that it is capable of being induced as desired by provision of the appropriate signal or stimulus. See, e.g., Published U.S. Patent Application No. 2005/0130184 A1, 16 Jun. 2005, Xu et al., directed to modified polymerase III promoters which utilize polymerase II enhancer elements, as well as Published U.S. Patent Application No. 2005/0130919 A1, 16 Jun. 2005, Xu et al., directed to regulatable polymerase III and polymerase II promoters, the teaching of which is hereby incorporated by reference.

By “protein” or “polypeptide” or “polypeptide fragment” is meant any chain of more than two amino acids, regardless of post-translational modification (e.g., glycosylation or phosphorylation), constituting all or part of a naturally-occurring polypeptide or peptide, or constituting a non-naturally occurring polypeptide or peptide.

By “reporter gene” is meant any gene that encodes a product whose expression is detectable and/or able to be quantitated by immunological, chemical, biochemical, or biological assays. A reporter gene product may, for example, have one of the following attributes, without restriction: fluorescence (e.g., green fluorescent protein), enzymatic activity (e.g., β-galactosidase, luciferase, chloramphenicol acetyltransferase), toxicity (e.g., ricin A), or an ability to be specifically bound by an additional molecule (e.g., an unlabeled antibody, followed by a labelled secondary antibody, or biotin, or a detectably labelled antibody). It is understood that any engineered variants of reporter genes that are readily available to one skilled in the art, are also included, without restriction, in the foregoing definition.

By “ribozyme” is meant a catalytic RNA polynucleotide capable of catalyzing RNA cleavage at a specific sequence. Ribozymes are useful, e.g., for attacking particular mRNA molecules. For example, in chronic myelogenous leukemia, a chromosomal translocation involving the genes bcr and abl (Philadelphia chromosome) results in expression of a bcr-abl fusion protein, which is believed to result in abnormal function of the abl oncoprotein. Because the fusion between the bcr and abl genes occurs at points within one of two introns, the spliced bcr-abl fusion transcript contains only two possible sequences at the splice junction between the bcr and abl exons. As the bcr-abl mRNA will only occur in lymphoid cells which have undergone this oncogenic chromosome translocation, a ribozyme specific for either of the two bcr-abl fusion mRNA splice junctions may be prepared, and thus may inhibit expression of the corresponding oncoprotein. See U.S. Pat. No. 6,080,851, “Ribozymes with linked anchor sequences”, Pachuk et al.

By “selective conditions” is meant conditions under which a specific cell or group of cells can undergo selection. For example, the parameters of a fluorescence-activated cell sorter (FACS) can be modulated to identify a specific cell or group of cells. Cell panning, a technique known to those skilled in the art, is another method that employs selective conditions.

By “short dsRNA” is meant a dsRNA that has 45, 40, 35, 30, 27, 25, 23, 21, 20, 19, 18, minimally 17 nucleotides in length that are in a double stranded conformation. Desirably, the short dsRNA is at least 19 basepairs in length. In desirable embodiments, the double stranded region is between 19 to 45, 19 to 40, 19 to 30, 19 to 20, 19 to 25, 20 to 25, 21 to 23, 25 to 30, or 30 to 40 contiguous basepairs in length, inclusive. In some embodiments, the short dsRNA is between 38 to 50, 50 to 100, 100 to 200, 200 to 300, 400 to 500, 500 to 700, 700 to 1000, 1000 to 2000, or 2000 to 5000 nucleotides in total length, inclusive and has one or more double stranded regions that are, independently, between 17, 18, or 19 and 45 contiguous basepairs in length, inclusive. In one embodiment, the short dsRNA is completely double stranded. In some embodiments, the short dsRNA is between 19 and 30 basepairs in length, and the entire dsRNA is double stranded. In other embodiments, the short dsRNA has one or two single stranded regions. In particular embodiments, the short dsRNA binds PKR (protein kinase RNA inducible) or another protein in a dsRNA-mediated stress response pathway. Desirably, the short dsRNA inhibits the dimerization and activation of PKR by at least 20, 40, 60, 80, 90, or 100%. In some desirable embodiments, the short dsRNA inhibits the binding of a long dsRNA to PKR or another component of a dsRNA-mediated stress response pathway by at least 20, 40, 60, 80, 90, or 100%.

It is recognized that shRNAs or short dsRNA hairpins may advantageously be expressed by polymerase III promoters, which can conveniently be used to express RNA molecules of less than approximately 400 to 500 nucleotides (nt) in length, preferably less than 100 to 200 nt in length. Accordingly, short dsRNA hairpins expressed by polymerase III may include a stretch of at least about 15 to 100 nucleotides (preferably 17 to 50 nt; more preferably 19 to 29 nt) capable of base pairing with a complementary sequence located on the same RNA molecule. The sequence and complementary sequence of the RNA molecule may be separated by an unpaired region of at least about 4 to 7 nucleotides (preferably about 9 to about 15 nucleotides) which forms a single-stranded loop above the stem structure created by the two regions of base complementarity. The shRNA molecules comprise at least one stem-loop structure comprising a double-stranded stem region of about 17 to about 100 bp; about 17 to about 50 bp; about 40 to about 100 bp; about 18 to about 40 bp; or from about 19 to about 29 bp; homologous and complementary to a target sequence to be inhibited; and an unpaired loop region of at least about 4 to 7 nucleotides; preferably about 9 to about 15 nucleotides, which forms a single-stranded loop above the stem structure created by the two regions of base complementarity. Included shRNAs are dual or bi-finger and multi-finger hairpin dsRNAs, in which the RNA molecule comprises two or more of such stem-loop structures separated by single-stranded spacer regions. Applicants intend that an expression construct of the invention may express multiple copies of the same, and/or one or more, including multiple different, short hairpin RNA molecules. Short hairpin RNA molecules considered to be the “same” as each other are those that comprise only the same double-stranded sequence, and short hairpin RNA molecules considered to be “different” from each other will comprise different double-stranded sequences, regardless of whether the sequences to be targeted by each different double-stranded sequence are within the same, or a different gene, such as, e.g., sequences of a promoter region and of a transcribed region (mRNA) of the same gene, or sequences of two different genes.

By “specifically hybridizes” is meant a dsRNA or other sequence-specific nucleic acid that hybridizes to a target nucleic acid molecule but does not substantially hybridize to other nucleic acid molecules in a sample (e.g., a sample from a cell) that naturally includes the target nucleic acid molecule, when assayed under denaturing conditions. In one embodiment, the amount of a target nucleic acid molecule hybridized to, or associated with, the dsRNA, as measured using standard assays, is 2-fold, desirably 5-fold, more desirably 10-fold, and most desirably 50-fold greater than the amount of a control nucleic acid molecule hybridized to, or associated with, the dsRNA.

By “specifically inhibits the expression of a target nucleic acid molecule” is meant that inhibition of the expression of a target nucleic acid molecule in a cell or biological sample occurs to a greater extent than the inhibition of expression of a non-target nucleic acid molecule that has a sequence that is less than 99, 95, 90, 80, or 70% identical or complementary to that of the target nucleic acid molecule. Desirably, the inhibition of expression of the non-target molecule is 2-fold, desirably 5-fold, more desirably 10-fold, and most desirably 50-fold less than the inhibition of expression of the target nucleic acid molecule.

An indication that nucleotide sequences are substantially identical is if two molecules hybridize to each other under stringent conditions. Stringent conditions are sequence-dependent and will be different in different circumstances. Generally, stringent conditions are selected to be about 5° C. to about 20° C., usually about 10° C. to about 15° C., lower than the thermal melting point (Tm) for the specific sequence at a defined ionic strength and pH. The Tm is the temperature (under defined ionic strength and pH) at which 50% of the target sequence hybridizes to a perfectly matched probe. Typically, stringent conditions will be those in which the salt concentration is about 0.02 molar at pH 7 and the temperature is at least about 60° C. For instance in a standard Southern hybridization procedure, stringent conditions will include an initial wash in 6×SSC at 42° C. followed by one or more additional washes in 0.2×SSC at a temperature of at least about 55° C., typically about 60° C. and often about 65° C.

By “substantial sequence complementarity” is meant sufficient sequence complementarity between a dsRNA, or other biologically active nucleic acid, and a target nucleic acid molecule for the nucleic acid to inhibit the expression of the target nucleic acid molecule. Preferably, the sequence of the dsRNA is at least 40, 50, 60, 70, 80, 90, 95, or 100% complementary to the sequence of a region of the target nucleic acid molecule.

By “substantial sequence identity” is meant sufficient sequence identity between a dsRNA or antisense RNA and a target nucleic acid molecule for the dsRNA to inhibit the expression of the nucleic acid molecule. Preferably, the sequence of the dsRNA or antisense RNA is at least 40, 50, 60, 70, 80, 90, 95, or 100% identical to the sequence of a region of the target nucleic acid molecule.

By “target”, “target nucleic acid”, “target gene”, “target polynucleotide” or “target polynucleotide sequence” is meant any nucleic acid sequence present in a eukaryotic cell, plant or animal, vertebrate or invertebrate, mammalian, avian, etc., whether a naturally-occurring, and possibly defective, polynucleotide sequence, or a heterologous sequence present due to an intracellular or extracellular pathogenic infection or a disease, whose expression is modulated as a result of post-transcriptional gene silencing, transcriptional gene silencing, or other sequence-specific dsRNA or RNA-mediated inhibition, such as antisense, ribozymal cleavage, etc. As used herein, the “target”, “target nucleic acid”, “target gene”, or “target polynucleotide sequence” may be in the cell in which the PTGS, transcriptional gene silencing (TGS), or other gene silencing event occurs, or it may be in a neighboring cell, or in a cell contacted with media or other extracellular fluid in which the cell that has undergone the PTGS, TGS, or other gene silencing event is contained. Such a “target”, “target nucleic acid”, “target gene”, or “target polynucleotide sequence” may be a coding sequence, that is, it is transcribed into an RNA, including an mRNA, whether or not it is translated to express a protein or a functional fragment thereof. Alternatively, it may be non-coding, but may have a regulatory function, including a promoter, enhancer, repressor, or any other regulatory element. The term “gene” is intended to include any target sequence intended to be “silenced”, whether or not transcribed and/or translated, including regulatory sequences, such as promoters.

Exemplary “target”, “target nucleic acid”, “target gene”, or “target polynucleotide sequence” molecules include nucleic acid molecules associated with cancer or abnormal cell growth, such as oncogenes, and nucleic acid molecules associated with an autosomal dominant or recessive disorder (see, for example, WO 00/63364, WO 00/44914, and WO 99/32619). Desirably, the antisense RNA, triplex forming RNA, or dsRNA inhibits the expression of an allele of a nucleic acid molecule that has a mutation associated with a dominant disorder and does not substantially inhibit the other allele of the nucleic acid molecule (e.g., an allele without a mutation associated with the disorder). Other exemplary “target”, “target nucleic acid”, “target gene”, or “target polynucleotide sequence” molecules include host cellular nucleic acid molecules and pathogen nucleic acid molecules including coding and non-coding regions required for the infection or propagation of a pathogen, such as a virus, bacteria, yeast, protozoa, or parasite.

By “target polypeptide” is meant a polypeptide whose biological activity is modulated by a therapeutic RNA molecule as a result of gene silencing or other sequence-specific RNA mediated mechanism, including antisense, ribozymal cleavage, etc. As used herein, the target polypeptide may be in the cell in which the PTGS, TGS, or other sequence-specific modulation occurs, or it may be in a neighboring cell, or in a cell contacted with media or other extracellular fluid in which the cell that has undergone the PTGS, TGS, or other gene silencing event is contained.

By “transformation” or “transfection” is meant any method for introducing foreign molecules into a cell (e.g., a bacterial, yeast, fungal, algal, plant, insect, or animal cell, particularly a vertebrate or mammalian cell). Lipofection, DEAE-dextran-mediated transfection, microinjection, protoplast fusion, calcium phosphate precipitation, viral or retroviral delivery, electroporation, and biolistic transformation are just a few of the transformation/transfection methods known to those skilled in the art. The RNA or RNA expression vector may be naked RNA or DNA or local anesthetic complexed RNA or DNA (Pachuk et al., Biochim. Biophys. Acta 1468:20-30 (2000)). Other standard transformation/transfection methods and other RNA and/or DNA delivery agents (e.g., a cationic lipid, liposome, or bupivacaine) are described in WO 00/63364, filed Apr. 19, 2000 (see, for example, pages 18-26). The dsRNAs or dsRNA expression constructs may also be complexed with the multifunctional molecular complexes of U.S. Pat. No. 5,837,533; U.S. Pat. No. 6,127,170; or U.S. Pat. No. 6,379,965 (Boutin), or the multifunctional molecular complexes or oil/water cationic amphiphile emulsions of PCT/US03/14288, filed May 6, 2003 (Satishchandran). Commercially available kits can also be used to deliver RNA or DNA to a cell. For example, the Transmessenger Kit from Qiagen, Inc. (Valencia, Cal.), an RNA kit from Xeragon Inc. (available from Qiagen), and an RNA kit from DNA Engine Inc. (Seattle, Wash.) can be used to introduce single or dsRNA into a cell.

By “transformed cell” or “transfected cell” is meant a cell (or a descendent of a cell) into which a nucleic acid molecule, for example, a dsRNA or double stranded expression vector, has been introduced, by means of recombinant nucleic acid techniques. Such cells may be either stably or transiently transfected.

By “treating, stabilizing, or preventing cancer” is meant causing a reduction in the size of a tumor, slowing or preventing an increase in the size of a tumor, increasing the disease-free survival time between the disappearance of a tumor and its reappearance, preventing an initial or subsequent occurrence of a tumor, or reducing or stabilizing an adverse symptom associated with a tumor. In one embodiment, the percent of cancerous cells surviving the treatment is at least 20, 40, 60, 80, or 100% lower than the initial number of cancerous cells, as measured using any standard assay. Preferably, the decrease in the number of cancerous cells induced by administration of a composition of the invention is at least 2, 5, 10, 20, or 50-fold greater than the decrease in the number of non-cancerous cells. In yet another embodiment, the number of cancerous cells present after administration of a composition of the invention is at least 2, 5, 10, 20, or 50-fold lower than the number of cancerous cells present after administration of a vehicle control. Preferably, the methods of the present invention result in a decrease of 20, 40, 60, 80, or 100% in the size of a tumor as determined using standard methods. Preferably, at least 20, 40, 60, 80, 90, or 95% of the treated subjects have a complete remission in which all evidence of the cancer disappears. Preferably, the cancer does not reappear, or reappears after at least 5, 10, 15, or 20 years. In another desirable embodiment, the length of time a patient survives after being diagnosed with cancer and treated with a composition of the invention is at least 20, 40, 60, 80, 100, 200, or even 500% greater than (i) the average amount of time an untreated patient survives or (ii) the average amount of time a patient treated with another therapy survives.

By “treating, stabilizing, or preventing a disease or disorder” is meant preventing or delaying an initial or subsequent occurrence of a disease or disorder; increasing the disease-free survival time between the disappearance of a condition and its reoccurrence; stabilizing or reducing an adverse symptom associated with a condition; or inhibiting or stabilizing the progression of a condition. This includes prophylactic treatment, in which treatment before infection with an infectious agent, such as a virus, bacterium, or fungus, is established, prevents or reduces the severity or duration of infection. Preferably, at least 20, 40, 60, 80, 90, or 95% of the treated subjects have a complete remission in which all evidence of the disease disappears. In another embodiment, the length of time a patient survives after being diagnosed with a condition and treated using a method of the invention is at least 20, 40, 60, 80, 100, 200, or even 500% greater than (i) the average amount of time an untreated patient survives, or (ii) the average amount of time a patient treated with another therapy survives.

By “under conditions that inhibit or prevent an interferon response or a dsRNA stress response” is meant conditions that prevent or inhibit one or more interferon responses or cellular RNA stress responses involving cell toxicity, cell death, an anti-proliferative response, or a decreased ability of a dsRNA to carry out a PTGS or TGS event. These responses include, but are not limited to, interferon induction (both Type I and Type II), induction of one or more interferon stimulated genes, PKR activation, 2′5′-OAS (oligoadenylate synthetase) activation, and any downstream cellular and/or organismal sequelae that result from the activation/induction of one or more of these responses. By “organismal sequelae” is meant any effect(s) in a whole animal, organ, or more locally (e.g., at a site of injection) caused by the stress response. Exemplary manifestations include elevated cytokine production, local inflammation, and necrosis. Desirably the conditions that inhibit these responses are such that not more than 95%, 90%, 80%, 75%, 60%, 40%, or 25%, and most desirably not more than 10% of the cells undergo cell toxicity, cell death, or a decreased ability to carry out a PTGS, TGS, or another gene silencing event, compared to a cell not exposed to such interferon response inhibiting conditions, all other conditions being equal (e.g., same cell type, same transformation with the same dsRNA expression library).

Apoptosis, interferon induction, 2′5′-OAS activation/induction, PKR induction/activation, anti-proliferative responses, and cytopathic effects are all indicators for the RNA stress response pathway. Exemplary assays that can be used to measure the induction of an RNA stress response as described herein include a TUNEL assay to detect apoptotic cells, ELISA assays to detect the induction of alpha, beta and gamma interferon, ribosomal RNA fragmentation analysis to detect activation of 2′5′-OAS, measurement of phosphorylated eIF2a as an indicator of PKR activation, proliferation assays to detect changes in cellular proliferation, and microscopic analysis of cells to identify cellular cytopathic effects. Desirably, the level of an interferon response or a dsRNA stress response in a cell transformed with a dsRNA or a dsRNA expression vector is less than 20, 10, 5, or 2-fold greater than the corresponding level in a mock-transfected control cell under the same conditions, as measured using one of the assays described herein. In other embodiments, the level of an interferon response or a dsRNA stress response in a cell transformed with a dsRNA or a dsRNA expression vector using the methods of the present invention is less than 500%, 200%, 100%, 50%, 25%, or 10% greater than the corresponding level in a corresponding transformed cell that is not exposed to such interferon response inhibiting conditions, all other conditions being equal. Desirably, the dsRNA does not induce a global inhibition of cellular transcription or translation. Notably, Applicants have demonstrated that dsRNA molecules, including long dsRNA molecules (e.g., 600 bp), may be expressed intracellularly in adult stress-response competent mammalian cells without any evidence of their inducing an interferon, stress, or “panic” response.

By “viral infection” is meant the invasion of a host animal by a virus.

For example, the infection may include the excessive growth of viruses that are normally present in or on the body of an animal or growth of viruses that are not normally present in or on the animal. More generally, a viral infection can be any situation in which the presence of a viral population(s) is damaging to a host animal. Thus, an animal is “suffering” from a viral infection when an excessive amount of a viral population is present in or on the animal's body, or when the presence of a viral population(s) is damaging the cells or other tissue of the animal.

In desirable embodiments, the viral infection relevant to the methods of the invention is an infection by one or more of the following viruses: Hepatitis B, Hepatitis C, picornarirus, polio, human immunodeficiency virus (HIV), coxsacchie, herpes simplex virus Type 1 and 2, St. Louis encephalitis, Epstein-Barr, myxoviruses, JC, coxsakieviruses B, togaviruses, measles, paramyxoviruses, echoviruses, bunyaviruses, cytomegaloviruses, varicella-zoster, mumps, equine encephalitis, lymphocytic choriomeningitis, rhabodoviruses including rabies, simian virus 40, human polyoma virus, parvoviruses, papilloma viruses, primate adenoviruses, coronaviruses, retroviruses, Dengue, yellow fever, Japanese encephalitis virus, and/or BK. In some embodiments, the first dsRNA inhibits the expression of a viral nucleic acid in a cell or animal infected with a virus.

Particularly suitable for the therapeutic and prophylactic methods of the invention are DNA viruses or viruses that have an intermediary DNA stages. Among such viruses are included, without limitation, viruses of the species Retrovirus, Herpesvirus, Hepadenovirus, Poxvirus, Parvovirus, Papillornavirus, and Papovavirus. Specifically some of the more desirable viruses to treat with this method include, without limitation, HIV, HBV, herpes simplex virus (HSV), cytomegalovirus (CMV), human papillomavirus (HPV), (human T-lymphocyte virus (HTLV), and Epstein-Barr virus (EBV). The agent used in this method provides to the cell of the mammal an at least partially double stranded RNA molecule as described herein, which includes a double-stranded sequence substantially homologous to an at least 17 to 19 contiguous nucleotide sequence of a target gene, including a coding sequence of a target polynucleotide which is a virus polynucleotide sequence necessary for replication and/or pathogenesis of the virus in an infected mammalian cell. Among such target polynucleotide sequences are protein encoding sequences for proteins necessary for the propagation of the virus, e.g., the HIV gag, env, and pol genes, the HPV6 LI and E2 genes, the HPV 11 LI and E2 genes, the HPV 16 E6 and E7 genes, the BPV 18 E6 and E7 genes, the HBV surface antigens, the HBV core antigen, HBV reverse transcriptase, the HSV gD gene, the HSVvp 16 gene, the HSV gC, gH, gL and gB genes, the HSV ICPO, ICP4 and ICP6 genes, Varicella zoster gB, gC and gH genes, and the BCR-abl chromosomal sequences, and non-coding viral polynucleotide sequences which provide regulatory functions necessary for transfer of the infection from cell to cell, e.g., the HIV LTR, and other viral promoter sequences, such as HSV vp 16 promoter, HSV-ICPO promoter, HSV-ICP4, ICP6 and gD promoters, the HBV surface antigen promoter, the HBV pre-genomic promoter, among others.

Thus, this method can be used to treat mammalian subjects already infected with a virus, such as HIV, in order to shut down or inhibit a viral gene function essential to virus replication and/or pathogenesis, such as HIV gag. Alternatively, this method can be employed to inhibit the functions of viruses which exist in mammals as latent viruses, e.g., Varicella zoster virus, and are the causative agents of the disease known as shingles. Similarly, diseases such as atherosclerosis, ulcers, chronic fatigue syndrome, and autoimmune disorders, recurrences of HSV-I and HSV-2, HPV persistent infection, e.g., genital warts, and chronic BBV infection among others, which have been shown to be caused, at least in part, by viruses, bacteria, or another pathogen, can be treated according to this method by targeting certain viral polynucleotide sequences essential to viral replication and/or pathogenesis in the mammalian subject.

Still another analogous embodiment of the above “anti-viral” methods of the invention includes a method for treatment or prophylaxis of a virally induced cancer in a mammal. Such cancers include HPV E6/E7 virus-induced cervical carcinoma, HTLV-induced cancer, and EBV induced cancers, such as Burkitts lymphoma, among others. This method is accomplished by administering to the mammal a composition, as described herein, in which the target polynucleotide is a sequence encoding a tumor antigen or functional fragment thereof, or a non-expressed regulatory sequence, which antigen or sequence function is required for the maintenance of the tumor in the mammal. Among such sequences are included, without limitation, HPV16 E6 and E7 sequences and HPV 18 E6 and E7 sequences. Others may readily be selected by one of skill in the art. The composition is administered in an amount effective to reduce or inhibit the function of the antigen in the mammal, and preferably employs the composition components, dosages, and routes of administration as described herein.

Transcriptional and Post-Transcriptional Gene Silencing

Transcriptional gene silencing (TGS) is a phenomenon in which silencing of gene expression occurs at the level of RNA transcription. Double stranded RNA mediates TGS as well as post-transcriptional gene silencing (PTGS), but the dsRNA needs to be located in the nucleus (preferably, the nucleolus, more preferably, both the nuclear compartment and the nucleolus), and desirably is made in the nucleus in order to mediate TGS. PTGS occurs in the cytoplasm. A number of dsRNA structures and dsRNA expression vectors have been delineated herein that can mediate TGS, PTGS, or both. Various strategies for mediating TGS, PTGS, or both are summarized below.

All of the cytoplasmic dsRNA expression vectors described herein mediate PTGS because they generate dsRNA in the cytoplasm where the dsRNA can interact with target mRNA. Because some of the dsRNA made by these vectors translocate to the nucleus via a passive process (e.g., due to nuclear envelope degeneration and reformation during mitosis), these vectors are also expected to affect TGS at a low efficiency in dividing cells.

To enhance TGS, an expression vector may further be provided with a nuclear localization signal, which targets the selected polynucleotide sequences to the nucleus of the cell transfected with the molecule, where transcription occurs. Suitable nuclear localization signals are known to those of skill in the art and are not a limitation of the present invention [see, e.g., D. A. Dean, Exp. Cell Res. 230:293-302 (1997)].

RNA pol II vectors express RNA molecules in the nucleus with various abilities to enter the cytoplasm. If desired, one or more constitutive transport element (CTE) sequences can be added to enable cytoplasmic transport of the different effector RNA molecules (e.g., mRNAs, antisense RNA, hairpin or duplex dsRNAs) that are made in the nucleus by RNA pol II. A CTE can be used instead of and/or in addition to an intron and/or polyA sequence to facilitate transport. A desirable location for the CTE is near the 3′ end of the RNA molecules. If desired, multiple CTE sequences (e.g., 2, 3, 4, 5, 6, or more sequences can be used). A preferred CTE is from the Mason-Pfizer Monkey Virus (U.S. Pat. Nos. 5,880,276 and 5,585,263).

Vectors encoding a functional intron or CTE in combination with a polyadenylation signal more efficiently export dsRNA to the cytoplasm. Vectors with (i) only an intron or CTE and no polyadenylation signal, or (ii) with only a polyadenylation signal and no intron or CTE, export RNA to the cytoplasm with a lesser efficiency, resulting in less RNA in the cytoplasm and a lower efficiency for PTGS. Vectors encoding RNA without an intron, CTE, and polyadenylation signal result in RNA molecules that are the least efficiently transported to the cytoplasm. The lower the level of cytoplasmic transport of RNA, the more RNA retention in the nucleus and the higher efficiency with which TGS is induced. Therefore, all of these vectors induce PTGS and TGS with varying efficiencies according to the level of cytoplasmic transport and nuclear retention, respectively, as described above.

RNA pol III vectors, which can have one or more introns or no introns and can have a polyA tail or no polyA tail, encode RNA molecules that are made in the nucleus and are primarily retained in the nucleus. This nuclear RNA induces TGS. However, a percentage of the transcribed RNA reaches the cytoplasm and can therefore induce PTGS. For TGS induction, the dsRNA desirably contains a promoter, or a subset of a promoter sequence, and is retained in the nucleus. Alternatively, the dsRNA may contain only coding or UTR sequence, or may desirably contain a combination of coding or UTR sequence and promoter sequence. Such “fusion target” dsRNAs may contain, e.g., both a promoter sequence and a linked gene sequence to be targeted for concurrent TGS and PTGS. The multiple-compartment expression systems of the invention can ensure that such a “fusion target” sequence is expressed in all of the relevant compartments, e.g., cytoplasm, nucleus, and nucleolus, by use of the requisite compartment-specific promoters to initiate transcription. For PTGS, the dsRNA contains sequence derived from an RNA (e.g., coding or UTR sequence from an mRNA) and does not have to contain promoter sequence. In addition, more efficient PTGS is induced by vectors that enable cytoplasmic transcription or by vectors that result in more efficiently cytoplasmically transported RNA. If desired, PTGS and TGS can be induced simultaneously with a combination of these vectors using the methods described herein and techniques known to those skilled in the art. Small therapeutic RNA molecules such as antisense, dsRNAs including siRNAs, shRNAs, microRNAs, aptamers, triplex, and ribozymes are adaptable to expression by RNA polymerase I and RNA polymerase III promoter/polymerase systems endogenous to the host cell.

By “RNA polymerase III promoter” or “RNA pol III promoter” or “polymerase III promoter” or “pol III promoter” is meant any invertebrate, vertebrate, or mammalian promoter, e.g., human, murine, porcine, bovine, primate, simian, etc. that, in its native context in a cell, associates or interacts with RNA polymerase III to transcribe its operably linked gene, or any variant thereof, natural or engineered, that will interact in a selected host cell with an RNA polymerase III to transcribe an operably linked nucleic acid sequence. The RNA polymerase III promoters are adaptable to expression of short RNA transcripts encoding therapeutic RNA molecules, up to a maximum of about 400 to 500 nucleotides in length. One reason RNA Pol III promoters are especially desirable for expression of such small engineered therapeutic RNA transcripts is that RNA Pol III termination occurs efficiently and precisely at a short run of thymine residues in the DNA coding strand, without other protein factors, T₄ and T₅ being the shortest Pol III termination signals in yeast and mammals, with oligo (dT) terminators longer than T₅ being very rare in mammals. Accordingly, expression constructs of the invention will include an appropriate oligo (dT) termination signal, i.e., a sequence of 4, 5, 6 or more Ts, operably linked 3′ to each RNA Pol III promoter in the DNA coding strand. Preferred in some aspects are the Type III RNA polymerase III promoters including U6 promoters, H1 promoters, and 7SK promoters which exist in the 5′ flanking region, include TATA boxes, and lack internal promoter sequences. Internal promoters occur for the pol III 5S rRNA, tRNA or VA RNA genes. The 7SLRNA pol III gene contains a weak internal promoter and a sequence in the 5′ flanking region of the gene necessary for transcription. RNA pol III promoters include any higher eukaryotic, including any vertebrate or mammalian, promoter containing any sequence variation or alteration, either natural or produced in the laboratory, which maintains or enhances but does not abolish the binding of RNA polymerase III to said promoter, and which is capable of transcribing a gene or nucleotide sequence, either natural or engineered, which is operably linked to said promoter sequence. Pol III promoters for utilization in an expression construct for a particular application, e.g., to express therapeutic RNA molecules such as hairpin dsRNAs against a fish, bird, or invertebrate virus may advantageously be selected for optimal binding and transcription by the host cell RNA polymerase III, e.g., including avian pol III promoters in an expression construct designed to transcribe a plurality of hairpin dsRNAs against an avian virus such as West Nile Virus or avian influenza virus (H5N1) in avian host cells and utilize instead human or other mammalian pol III promoters in an expression construct designed to transcribe a plurality of hairpin dsRNAs against an avian virus such as West Nile Virus or avian influenza virus (H5N1) in human host cells.

Other expression control sequences in the expression constructs of the invention include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product.

It will be recognized that any of the expression constructs or vectors described herein or any other standard vector can be used to generate any of the desired biologically active nucleic acid structures of the invention, e.g., dsRNA, mRNA (translated if desired, into polypeptide), antisense RNA, and ribozymal RNA, and used in the present methods.

Methods for Enhancing Post-Transcriptional Gene Silencing

To enhance PTGS by dsRNA transcribed in the nucleus by RNA pol II, one or more introns and/or a polyadenylation signal can be added to the dsRNA to enable processing of the transcribed RNA. This processing is desirable because both splicing and polyadenylation facilitate export from the nucleus to the cytoplasm. In addition, polyadenylation stabilizes RNA pol II transcripts. These same strategies will be useful for expression of functional mRNAs that will be translated into protein. In some embodiments, a prokaryotic antibiotic resistance gene, e.g., a zeomycin expression cassette, is located in the intron. Other exemplary prokaryotic selectable markers include other antibiotic resistance genes such as kanamycin, including the chimeric kanamycin resistance gene of U.S. Pat. No. 5,851,804, aminoglycosides, tetracycline, and ampicillin. The zeomycin gene is under the regulatory control of a prokaryotic promoter, and translation of zeomycin in the host bacterium is ensured by the presence of Shine-Dalgarno sequences located within about 10 base-pairs upstream of the initiating ATG. Alternatively, the zeomycin expression cassette can be placed in any location between the inverted repeat sequences of the hairpin (i.e., between the sense and antisense sequences with substantial identity to the target nucleic acid to be silenced).

Although inverted repeat sequences are usually deleted from DNA by DNA recombination when a vector is propagated in bacteria, a small percentage of bacteria may have mutations in the recombination pathway that allow the bacteria to stably maintain DNA bearing inverted repeats. In order to screen for these infrequent bacteria, a zeomycin selection is added to the culture. The undesired bacteria that are capable of eliminating inverted repeats are killed because the zeomycin expression cassette is also deleted during recombination. Only the desired bacteria with an intact zeomycin expression cassette survive the selection.

After the DNA is isolated from the selected bacteria and inserted into eukaryotes (e.g., mammalian cell culture) or into animals (e.g., adult mammals) for expression of RNA, the intron is spliced from the RNA transcripts. If the zeomycin expression cassette is located in the intron, this cassette is removed by RNA splicing. In the event of inefficient splicing, the zeomycin expression cassette is not expressed because there are no eukaryotic signals for transcription and translation of this gene. The elimination of the antibiotic resistance cassette is desirable for applications involving short dsRNA molecules because the removal of the cassette decreases the size of the dsRNA molecules. The zeomycin cassette can also be located beside either end of an intron instead of within the intron. In this case, the zeomycin expression cassette remains after the intron is spliced and can be used to participate in the loop structure of the hairpin. These RNA pol II transcripts are made in the nucleus and transported to the cytoplasm where they can effect PTGS. However, some RNA molecules may be retained in the nucleus. These nuclear RNA molecules may effect TGS. For TGS applications, the encoded dsRNA desirably contains a promoter or a subset of a promoter. In order to more efficiently retain RNA within the nucleus, the intron and/or polyadenylation signal can be removed.

Another strategy for both cytoplasmic and nuclear localization is to use “upstream” or internal RNA pol III promoters (see, e.g., Gene regulation: A Eukaryotic Perspective, 3^{rd} ed., David Latchman (Ed.) Stanley Thornes: Cheltenham, UK, 1998). These promoters result in nuclear transcribed RNA transcripts, some of which are exported and some of which are retained in the nucleus and hence can be used for PTGS and/or TGS. These promoters can be used to generate hairpins, including the partial and forced hairpin structures of the invention, or duplex RNA through the use of converging promoters or through the use of a two vector or two cistronic system. One promoter directs synthesis of the sense strand, and the other promoter directs synthesis of the antisense RNA. The length of RNA transcribed by these promoters is generally limited to several hundred nucleotides (e.g., 250-500). In addition, transcriptional termination signals may be used in these vectors to enable efficient transcription termination.

Much of the nomenclature and general laboratory procedures required in this application can be found in Sambrook J. et al., Molecular Cloning: A Laboratory Manual (3rd Ed.), Vol. 1-3, Cold Spring Harbor Laboratory, Cold Spring Harbor, N.Y., 2000. The manual is hereinafter referred to as “Sambrook et al.”

Standard techniques for cloning, DNA isolation, amplification and purification, for enzymatic reactions involving DNA ligase, DNA polymerase, restriction endonucleases and the like, and various separation techniques are those known and commonly employed by those skilled in the art of molecular biology, as e.g., in the following reference texts, incorporated herein by reference. A number of standard techniques are described in Ausubel et al. (1994) Current Protocols in Molecular Biology, Green Publishing, Inc., and Wiley and Sons, New York, N.Y.; Sambrook et al. (above); Maniatis et al. (1982) Molecular Cloning, Cold Spring Harbor Laboratory, Plainview, N.Y.; Wu (ed.) (1993) Meth. Enzymol. 218, Part I; Wu (ed.) (1979) Meth Enzymol. 68; Wu et al. (eds.) (1983) Meth. Enzymol. 100 and 101; Grossman and Moldave (eds.) Meth. Enzymol. 65; Miller (ed.) (1972) Experiments in Molecular Genetics, Cold Spring Harbor Laboratory, Cold Spring Harbor, N.Y.; Old and Primrose (1981) Principles of Gene Manipulation, University of California Press, Berkeley; Schleif and Wensink (1982) Practical Methods in Molecular Biology; Glover (ed.) (1985) DNA Cloning Vol. I and II, IRL Press, Oxford, UK; Hames and Higgins (eds.) (1985) Nucleic Acid Hybridization, IRL Press, Oxford, UK; and Setlow and Hollaender (1979) Genetic Engineering: Principles and Methods, Vols. 1-4, Plenum Press, New York. A particularly useful technique employed herein, called “chain reaction cloning”, is described in U.S. Pat. No. 6,143,527, “Chain reaction cloning using a bridging oligonucleotide and DNA ligase”, Pachuk et al. Abbreviations and nomenclature, where employed, are deemed standard in the field and commonly used in professional journals such as those cited herein.

Pharmaceutical Compositions

The multi-compartment expression systems of the invention may advantageously be used for a variety of pharmaceutical applications as described elsewhere herein. In various embodiments, the pharmaceutical composition includes about 1 ng to about 20 mg of nucleic acid, e.g., RNA, DNA, plasmids, viral vectors, recombinant viruses, or mixtures thereof, which provide the desired amounts of the nucleic acid molecules (dsRNA homologous to a target nucleic acid, mRNAs, antisense RNA, triplex-forming RNA, etc.). In some embodiments, the composition contains about 10 ng to about 10 mg of nucleic acid, about 0.1 mg to about 500 mg, about 1 mg to about 350 mg, about 25 mg to about 250 mg, or about 100 mg of nucleic acid. Those of skill in the art of clinical pharmacology can readily arrive at such dosing schedules using routine experimentation.

Suitable carriers include, but are not limited to, saline, buffered saline, dextrose, water, glycerol, ethanol, and combinations thereof. The composition can be adapted for the mode of administration and can be in the form of, for example, a pill, tablet, capsule, spray, powder, or liquid. In some embodiments, the pharmaceutical composition contains one or more pharmaceutically acceptable additives suitable for the selected route and mode of administration. These compositions may be administered by, without limitation, any parenteral route including intravenous (IV), intra-arterial, intramuscular (IM), subcutaneous (SC), intradermal, intraperitoneal, intrathecal, as well as topically, orally, and by mucosal routes of delivery such as intranasal, inhalation, rectal vaginal, buccal, and sublingual. In some embodiments, the pharmaceutical compositions of the invention are prepared for administration to vertebrate (e.g., mammalian) subjects in the form of liquids, including sterile, non-pyrogenic liquids for injection, emulsions, powders, aerosols, tablets, capsules, enteric coated tablets, or suppositories.

A pharmaceutical composition can be prepared as described herein, e.g., comprising a DNA plasmid construct expressing, under the control of a bacteriophage T7 promoter, a dsRNA substantially homologous to, e.g., one or more genes from the smallpox virus and human cell receptor sequences for the Anthrax toxin. The T7 RNA polymerase can be co-delivered and expressed from the same or another plasmid under the control of a suitable promoter e.g., hCMV, simian CMV, or SV40. In some embodiments, the same or another construct expresses the target gene (e.g., a target smallpox gene) contemporaneously with the dsRNA homologous to the target smallpox gene. The pharmaceutical composition is prepared in a pharmaceutical vehicle suitable for the particular route of administration. For IM, SC, IV, intradermal, intrathecal or other parenteral routes of administration, a sterile, non-toxic, pyrogen-free aqueous solution such as Sterile Water for Injection, and, optionally, various concentrations of salts, e.g., NaCl, and/or dextrose, (e.g., Sodium Chloride Injection, Ringer's Injection, Dextrose Injection, Dextrose and Sodium Chloride Injection, and Lactated Ringer's Injection) is commonly used. Optionally, other pharmaceutically appropriate additives, preservatives, or buffering agents known to those in the art of pharmaceutics are also used. If provided in a single dose vial for injection, the dose will vary as determined by those of skill in the art of pharmacology, but may typically contain between 5 mcg to 500 mcg of the active construct. If deemed necessary, significantly larger doses may be administered without toxicity, e.g., up to 5-10 mg.

Pharmaceutical compositions of the present invention and for use in accordance with the present invention may include a pharmaceutically acceptable excipient or carrier. As used herein, the term “pharmaceutically acceptable carrier” means a non-toxic, inert solid, semi-solid or liquid filler, diluent, encapsulating material or formulation auxiliary of any type. Some examples of materials which can serve as pharmaceutically acceptable carriers are sugars such as lactose, glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols such as propylene glycol; esters such as ethyl oleate and ethyl laurate; agar; detergents such as Tween 80; buffering agents such as magnesium hydroxide and aluminum hydroxide; alginic acid; pyrogen-free water; isotonic saline; Ringer's solution; ethyl alcohol; and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

The injectable formulations can be sterilized, for example, by filtration through a bacteria-retaining filter, or by incorporating sterilizing agents in the form of sterile solid compositions which can be dissolved or dispersed in sterile water or other sterile injectable medium prior to use.

Compositions for rectal or vaginal administration are preferably suppositories which can be prepared by mixing the expression construct(s) with suitable non-irritating excipients or carriers such as cocoa butter, polyethylene glycol, or a suppository wax which are solid at ambient temperature but liquid at body temperature and therefore melt in the rectum or vaginal cavity and release the microparticles.

Solid dosage forms for oral administration include capsules, tablets, pills, powders, and granules. In such solid dosage forms, the expression construct(s) are mixed with at least one inert, pharmaceutically acceptable excipient or carrier such as sodium citrate or dicalcium phosphate and/or a) fillers or extenders such as starches, lactose, sucrose, glucose, mannitol, and silicic acid; b) binders such as, for example, carboxymethylcellulose, alginates, gelatin, polyvinylpyrrolidinone, sucrose, and acacia; c) humectants such as glycerol; d) disintegrating agents such as agar-agar, calcium carbonate, potato or tapioca starch, alginic acid, certain silicates, and sodium carbonate; e) solution retarding agents such as paraffin; f) absorption accelerators such as quaternary ammonium compounds; g) wetting agents such as, for example, cetyl alcohol and glycerol monostearate; h) absorbents such as kaolin and bentonite clay; and i) lubricants such as talc, calcium stearate, magnesium stearate, solid polyethylene glycols, sodium lauryl sulfate, and mixtures thereof. In the case of capsules, tablets, and pills, the dosage form may also comprise buffering agents.

Dosage forms for topical or transdermal administration of an inventive pharmaceutical composition include ointments, pastes, creams, lotions, gels, powders, solutions, sprays, inhalants, or patches. The expression construct(s) are admixed under sterile conditions with a pharmaceutically acceptable carrier and any needed preservatives or buffers as may be required. Ophthalmic formulation, eardrops, and eye drops are also contemplated as being within the scope of this invention.

Transdermal patches have the added advantage of providing controlled delivery of a compound to the body. Such dosage forms can be made by dissolving or dispensing the expression construct(s) in a proper medium. Absorption enhancers can also be used to increase the flux of the compound across the skin. The rate can be controlled by either providing a rate controlling membrane or by dispersing the expression construct(s) in a polymer matrix or gel.

Desirable Methods of Administration of Expression Constructs

The DNA and/or RNA constructs of the invention may be administered to the host cell/tissue/organism as “naked” DNA, RNA, or DNA/RNA, formulated in a pharmaceutical vehicle without any transfection promoting agent. More efficient delivery may be achieved as known to those of skill in the art of DNA and RNA delivery, using e.g., such polynucleotide transfection facilitating agents known to those of skill in the art of RNA and/or DNA delivery. The following are exemplary agents: cationic amphiphiles including local anesthetics such as bupivacaine, cationic lipids, liposomes, or lipidic particles; polycations such as polylysine; branched, three-dimensional polycations such as dendrimers; carbohydrates; detergents; or surfactants, including benzylammonium surfactants such as benzylkonium chloride. Non-exclusive examples of such facilitating agents or co-agents useful in this invention are described in U.S. Pat. Nos. 5,593,972; 5,703,055; 5,739,118; 5,837,533; 5,962,482; 6,127,170; and 6,379,965, as well as International Patent Application Nos. PCT/US03/14288, filed May 6, 2003 (multifunctional molecular complexes and oil/water cationic amphiphile emulsions), and PCT/US98/22841; the teaching of which is hereby incorporated by reference. U.S. Pat. Nos. 5,824,538; 5,643,771; and 5,877,159 (incorporated herein by reference) teach delivery of a composition other than a polynucleotide composition, e.g., a transfected donor cell or a bacterium containing the expression constructs of the invention.

In some embodiments, the expression construct(s) of the invention is complexed with one or more cationic lipids or cationic amphiphiles, such as the compositions disclosed in U.S. Pat. No. 4,897,355 (Eppstein et al., filed Oct. 29, 1987); U.S. Pat. No. 5,264,618 (Feigner et al., filed Apr. 16, 1991); or U.S. Pat. No. 5,459,127 (Feigner et al., filed Sep. 16, 1993). In other embodiments, the expression construct(s) is complexed with a liposome/liposomic composition that includes a cationic lipid and optionally includes another component, such as a neutral lipid (see, for example, U.S. Pat. No. 5,279,833 (Rose); U.S. Pat. No. 5,283,185 (Epand); and U.S. Pat. No. 5,932,241 (Gorman)). In other embodiments, the expression construct(s) are complexed with the multifunctional molecular complexes of U.S. Pat. No. 5,837,533; U.S. Pat. No. 6,127,170; and U.S. Pat. No. 6,379,965 (Boutin), or, desirably, the multifunctional molecular complexes or oil/water cationic amphiphile emulsions of U.S. Prov. 60/378,191 filed May 6, 2002 and PCT/US03/14288, filed May 6, 2003 (Satishchandran), the teaching of which is incorporated herein by reference. The lafter application teaches a composition that includes a nucleic acid, an endosomolytic spermine that includes a cholesterol or fatty acid, and a targeting spermine that includes a ligand for a cell surface molecule. The ratio of positive to negative charge of the composition is between 0.5 and 1.5, inclusive; the endosomolytic spermine constitutes at least 20% of the spermine-containing molecules in the composition; and the targeting spermine constitutes at least 10% of the spermine-containing molecules in the composition. Desirably, the ratio of positive to negative charge is between 0.8 and 1.2, inclusive, such as between 0.8 and 0.9, inclusive. The targeting spermine is designed to localize the composition to a particular cell or tissue of interest. The endosomolytic spermine disrupts the endosomal vesicle the encapsulates the composition during endocytosis, facilitating release of the nucleic acid from the endosomal vesicle and into the cytoplasm or nucleus of the cell.

In yet other embodiments, the expression construct(s) is complexed with any other composition that is devised by one of ordinary skill in the fields of pharmaceutics and molecular biology. In some embodiments, the construct or vector is not complexed with a cationic lipid.

Transformation/transfection of the cell may occur through a variety of means including, but not limited to, lipofection, DEAE-dextran-mediated transfection, microinjection, protoplast fusion, calcium phosphate precipitation, viral or retroviral delivery, electroporation, or biolistic transformation. The expression construct (DNA) may be naked DNA or local anesthetic complexed DNA (Pachuk et al., Biochim. Biophys. Acta 1468:20-30 (2000)). Desirably the eukaryotic cell, e.g., vertebrate (e.g., mammalian), is in vivo or is a cell that has been cultured for only a small number of passages (e.g., less than 30 passages of a cell line that has been directly obtained from American Type Culture Collection), or are primary cells.

Desirable Cells

In still further embodiments of any aspect of the invention, the cell is a eukaryotic plant cell or an animal cell. Desirably the animal cell is an invertebrate or vertebrate cell (e.g., a mammalian cell, for example, a human cell). The cell may be ex vivo or in vivo. The cell may be a gamete or a somatic cell, for example, a cancer cell, a stem cell, a cell of the immune system, a neuronal cell, a muscle cell, or an adipocyte. In some embodiments, one or more proteins involved in gene silencing, such as Dicer or Argonaut, are overexpressed or activated in the cell or animal to increase the amount of inhibition of gene expression.

Inclusion of a Mammalian Origin of Replication

An origin of replication enables the DNA plasmid to be replicated upon nuclear localization and thus enhances expression. The advantage is that more plasmid is available for nuclear transcription and therefore more effector molecules are made (e.g., more antisense, mRNA, dsRNA hairpins and/or more dsRNA duplexes). Many origins are species-specific and work in several mammalian species but not in all species. For example, the SV40 T origin of replication (e.g., from plasmid pDsRed1-Mito from Clontech; U.S. Pat. No. 5,624,820) is functional in mice but not in humans. This origin can thus be used for vectors that are used or studied in mice. Other origins that can be used for human applications, such as the Epstein-Barr nuclear antigen (EBNA) origin (e.g., plasmids pSES.Tk and pSES.B from Qiagen). DNA vectors containing these elements are commercially available, and the DNA segment encoding the origin can be obtained using standard methods by isolating the restriction fragment containing the origin or by PCR amplifying the origin. The restriction maps and sequences of these vectors are available publicly and enable one skilled in the art to amplify these sequences or isolate the appropriate restriction fragment. These vectors replicate in the nuclei of cells that express the appropriate accessory factors such as SV40 TAg and EBNA. The expression of these factors is easily accomplished because some of the commercially available vectors (e.g., pSES.Tk and pSES.B from Qiagen) that contain the corresponding origin of replication also express either SV40 Tag or the EBNA. These DNA molecules containing the origin of replication can be easily cloned into a vector of interest (e.g., a vector expressing a dsRNA such as a hairpin or duplex) by one skilled in the art. These vectors are then co-transfected, injected, or administered with a vector expressing EBNA or Tag to enable replication of the plasmid bearing the EBNA or Tag origin of replication, respectively. Alternatively, the genes encoding EBNA or Tag are cloned into any another expression vector designed to work in the cells, animal, or organism of interest using standard methods. The genes encoding EBNA and Tag can also be cloned into the same vector bearing the origin of replication. Suitable origins of replication are not limited to Tag and EBNA; for example, Replicor in Montreal has identified a 36 base-pair mammalian origin consensus sequence that permits the DNA sequence to which it is attached to replicate (as reviewed in BioWorld Today, Aug. 16, 1999, Volume 10, No. 157). This sequence does not need the co-expression of auxiliary sequences to enable replication.

Also included within the scope of the invention are kits comprising compositions containing the expression constructs of the invention; wherein such kits may also optionally comprise media, solutions, and other compositions to assist in the stability, delivery, ease of use, or efficacy of the expression constructs.

### EXAMPLES

The present invention is further defined in the following Examples. It should be understood that these Examples, while indicating preferred embodiments of the invention, are given by way of illustration only. From the above discussion and these Examples, one skilled in the art can ascertain the preferred features of this invention, and without departing from the spirit and scope thereof, can make various changes and modifications of the invention to adapt it to various uses and conditions.

### Example 1

A Plasmid Multi-Compartment Eukaryotic Expression System Encoding Two Copies Each of Two Different HBV-Derived Hairpin dsRNAs, Each Located within a Separate Cistron.

A plasmid is constructed which encodes two copies each of two different HBV-specific hairpin RNAs (an RNA strand capable of assuming a double stranded structure by virtue of comprising inverted sense and antisense sequences separated by a “loop” sequence). Each such sequence, Sequence A and Sequence B, is under the control of two separate and distinct promoters, each transcriptionally active in a different subcellular compartment of a eukaryotic cell: the bacteriophage T7 promoter (T7p) (which promotes transcription by T7 RNA polymerase in the cytoplasm) and the human U6 promoter (an RNA pol III promoter transcriptionally active in the nucleolus). The transcription units (cistrons) are arranged such that there is a separate location for each cistron (FIG. 7) for a total of four cistrons. The T7 promoter can be one of the different versions supplied in commercially available vectors, but the sequence of the one described in this and the following examples is as follows (5′ to 3′): 5′TAATACGACTCACTATAGGG3′ (SEQ ID NO:1). Note that the definition of the T7 promoter includes one, two or three G residues (shown in italics) positioned at the +1 and +2 and +3 site to ensure efficient transcription initiation. These Gs are positioned immediately at the 3′ terminus of the promoter such that they are in the +1, or +1 and +2, or +1, +2 and +3 positions relative to the “core” nucleotides of the T7 promoter. A T7 terminator (T7t) (Lyakhov et al. [1]) can be included at the end of each T7 cistron as indicated in FIG. 7.

Vector description: The first hairpin (Sequence A) maps to coordinates 2905-2929 of GenBank Accession #s V01460 and J02203 (i.e., the hairpin contains the sense and antisense versions of this sequence, separated by a loop structure of TTCAAAAGA). Description of U6-based vector systems can be found in Lee et al. [2]. The second vector encoded hairpin (Sequence B) maps to coordinates 1215-1239 of GenBank Accession #s V01460 and J02203. Like the first hairpin, it encodes the sense and antisense versions of this sequence, separated by a loop structure of TTCAAAAGA. This vector is assessed in an HBV replicon model described below. Cloning is performed using standard techniques or if desired directional ligation cloning, CRC, can be used [3] (See the teaching of U.S. Pat. No. 6,143,527, “Chain reaction cloning using a bridging oligonucleotide and DNA ligase”, Pachuk et al., incorporated herein by reference.) For this experiment, a T7 RNA polymerase expression plasmid is co-transfected with the experimental plasmid so as to provide a source of T7 RNA polymerase. This vector is described near the end of this example.

HBV Replicon Model: Silencing HBV Replication and Expression in a Replication Competent Cell Culture Model.

Brief description of cell culture model: A human liver-derived cell line such as the Huh7 cell line is transfected with an infectious molecular clone of HBV consisting of a terminally redundant viral genome that is capable of transcribing all of the viral RNAs and producing infectious virus [4-6]. The replicon used in these studies is derived from the virus sequence found in Gen Bank Accession #s V01460 and J02203. Following internalization into hepatocytes and nuclear localization, transcription of the infectious HBV plasmid from several viral promoters has been shown to initiate a cascade of events that mirror HBV replication. These events include translation of transcribed viral mRNAs, packaging of transcribed pregenomic RNA into core particles, reverse transcription of pregenomic RNA, and assembly and secretion of virions and HBsAg (Hepatitis B Surface Antigen) particles into the media of transfected cells. This transfection model reproduces most aspects of HBV replication within infected liver cells and is therefore a good cell culture model with which to look at silencing of HBV expression and replication.

In this model, cells are co-transfected with the infectious molecular clone of HBV and the effector RNA constructs to be evaluated: The cells are then monitored for loss of HBV expression and replication as described below.

Experimental Procedure: Transfection. Huh7 cells are seeded into six-well plates such that they are between 80-90% confluency at the time of transfection. All transfections are performed using Lipofectamine™ polycationic lipid/neutral lipid liposome formulation (Invitrogen) according to the manufacturer's directions. In this experiment, cells are transfected with 50 ng of the infectious HBV plasmid, 1 μg of a T7 RNA polymerase expression plasmid (description of plasmid below) and 1.5 μg of the experimental plasmid of Example 1, depicted in FIG. 7. Control cells are transfected with 50 ng of the HBV plasmid and 1 μg of the T7 RNA polymerase expression plasmid. An inert filler DNA, pGL3-basic (Promega, Madison Wis.), is added to all transfections to bring total DNA/transfection up to 2.5 μg DNA.

Monitoring cells for loss of HBV expression. Following transfection, cells are monitored for the loss or reduction in HBV expression and replication by measuring HBsAg secretion and DNA-containing viral particle secretion. Cells are monitored by assaying the media of transfected cells beginning at 2 days post dsRNA administration and every other day thereafter for a period of three weeks. The Auszyme ELISA, commercially available from Abbott Labs (Abbott Park, Ill.), is used to detect HBV surface Ag (sAg). sAg is measured since surface Ag is associated not only with viral replication but also with RNA polymerase II initiated transcription of the surface Ag cistron in the transfected infectious HBV clone. Since surface Ag synthesis can continue in the absence of HBV replication, and since continued production of surface antigen in chronic HBV infection is associated with the development of hepatocellular carcinoma, it is important to down-regulate not only viral replication but also replication-independent synthesis of sAg. Secretion of virion particles containing encapsidated HBV genomic DNA is also measured. Loss of virion particles containing encapsidated DNA is indicative of a loss of HBV replication.

Analysis of virion secretion involves a technique that discriminates between naked, immature core particles and enveloped infectious HBV virions [7]. Briefly, pelleted viral particles from the media of cultured cells are subjected to Proteinase K digestion to degrade the core proteins. Following inactivation of Proteinase K, the sample is incubated with RQ1 DNase (Promega, Madison, Wis.) to degrade the DNA liberated from core particles. The sample is digested again with Proteinase K in the presence of SDS to inactivate the DNase as well as to disrupt and degrade the infectious enveloped virion particle. DNA is then purified by phenol/chloroform extraction and ethanol precipitated. HBV specific DNA is detected by gel electrophoresis followed by Southern Blot analysis.

Predicted Results indicate a 70-95% decrease in both sAg and viral particle secretion in the media of cells transfected with the HBV plasmid, T7 RNA polymerase expression plasmid and experimental plasmid relative to cells transfected with only the HBV plasmid T7. RNA polymerase expression plasmid and filler DNA.

Vectors Used in Experiment

Sequence of the T7 RNA Polymerase Gene:

SEQ ID NO:2 is the sequence of the T7 RNA polymerase gene which is cloned into a mammalian expression vector such as pCEP4 (Invitrogen, Carlsbad, Calif.). Cloning can be easily done by one skilled in the art. One skilled in the art would also be aware that a leader sequence with a Kozak sequence needs to be cloned in directly upstream from the T7 RNA polymerase gene.

### Example 2

A Vector Encoding an HBV-Derived Hairpin RNA, in the Flanked Promoter Arrangement.

A plasmid is constructed which encodes an HBV-specific hairpin RNA (Sequence A from Example 1), under the control of two separate, different compartment promoters: the bacteriophage T7 promoter (cytoplasmic, when T7 RNA polymerase is also supplied) and the human U6 promoter (RNA pol III, nucleolar). The sequence is transcribed in one direction by the T7 promoter and in the reverse direction by the U6 promoter. A T7 terminator is cloned at the 3′ end of Sequence A, relative to the T7 promoter, and a U6 terminator is cloned at the 3′ end of Sequence A, relative to the U6 promoter. The T7 transcript will contain from 5′ to 3′: the reverse complement to the U6 terminator and the Sequence A hairpin (e.g., sense-loop-antisense). The U6 transcript will contain from 5′ to 3′: the reverse complement to the T7 terminator, and the Sequence A hairpin (e.g., antisense-loop-sense) Although the HBV-hairpin sequence encoded within the T7 transcript is the reverse complement of that encoded by the U6 transcript, both transcripts contain the same sense and antisense HBV sequences in double-stranded conformation and are thus expected to be functionally equivalent with respect to HBV-specific silencing. Two, three, four, five or more of such “flanked promoter cistrons” expressing, e.g. two, three, four or more different therapeutic RNA molecules, e.g., hairpin dsRNA molecules, active against different regions of a selected target gene or different target gene sequences of a single target pathogen or against genes of multiple different target pathogens, may be included in a single expression construct. Expression constructs comprising sets of two of such flanked promoter cistrons (e.g., cytoplasmic/pol III (e.g. T7/7SK), pol/pol I, pol II/pol III) expressing a sense sequence and an antisense sequence are readily adapted to expression of duplex dsRNA molecules.

In an alternative embodiment, not utilizing a flanked promoter arrangement, an expression vector is constructed comprising a sequence encoding a therapeutic RNA molecule such as a dsRNA hairpin (e.g., as in Sequence A) operably linked to a polymerase III promoter (e.g., a type III RNA pol III promoter such as U6, H1, 7SK) as well as another sequence encoding a therapeutic RNA molecule such as a dsRNA hairpin, the same or different from Sequence A, operably linked to a different category of promoter, e.g., a promoter such as T7, SP6, or SP3 active in the cytoplasm, and/or a sequence encoding a therapeutic RNA molecule linked to a pol II promoter active in a distinct functional location in the nucleus, and/or a sequence encoding a therapeutic RNA molecule linked to a polymerase I promoter active in the nucleolus. Appropriate elements such as terminators will be included as known to those of skill in the art.

Vector description: The hairpin (Sequence A) maps to coordinates 2905-2929 of GenBank Accession #s V01460 and J02203 (i.e., the hairpin contains the sense and antisense version of this sequence, separated by a loop structure of TTCAAAAGA). Cloning is performed using standard techniques or if desired directional ligation cloning, CRC, can be used [3] as in U.S. Pat. No. 6,143,527, “Chain reaction cloning using a bridging oligonucleotide and DNA ligase”, Pachuk et al., incorporated herein by reference. The vector is assessed in an HBV replicon model. For this experiment, a T7 RNA polymerase expression plasmid (as described in Example 1 above) is co-transfected with the experimental plasmid so as to provide a source of T7 RNA polymerase.

HBV Replicon Model: Silencing HBV Replication and Expression in a Replication Competent Cell Culture Model.

Brief description of cell culture model: A human liver derived cell line such as the Huh7 cell line is transfected with an infectious molecular clone of HBV consisting of a terminally redundant viral genome that is capable of transcribing all of the viral RNAs and producing infectious virus [4-6]. The replicon used in these studies is derived from the virus sequence found in GenBank Accession #s V01460 and J02203. Following internalization into hepatocytes and nuclear localization, transcription of the infectious HBV plasmid from several viral promoters has been shown to initiate a cascade of events that mirror HBV replication. These events include translation of transcribed viral mRNAs, packaging of transcribed pregenomic RNA into core particles, reverse transcription of pregenomic RNA, and assembly and secretion of virions and HBsAg particles into the media of transfected cells. This transfection model reproduces most aspects of HBV replication within infected liver cells and is therefore a good cell culture model with which to look at silencing of HBV expression and replication.

In this model, cells are co-transfected with the infectious molecular clone of HBV and the effector RNA constructs to be evaluated. The cells are then monitored for loss of HBV expression and replication as described below.

Experimental Procedure: Transfection. Huh7 cells are seeded into six-well plates such that they are between 80-90% confluency at the time of transfection. All transfections are performed using Lipofectamine™ (Invitrogen) according to the manufacturer's directions. In this experiment, cells are transfected with 50 ng of the infectious HBV plasmid, 1 μg of a T7 RNA polymerase expression plasmid (description of plasmid below) and 1.5 μg of the experimental plasmid depicted in FIG. 8. Control cells are transfected with 50 ng of the HBV plasmid and 1 μg of the T7 RNA polymerase expression plasmid. An inert filler DNA, pGL3-basic (Promega, Madison Wis.), is added to all transfections to bring total DNA/transfection up to 2.5 μg DNA.

Monitoring cells for loss of HBV expression. Following transfection, cells are monitored for the loss or reduction in HBV expression and replication by measuring HBsAg secretion and DNA-containing viral particle secretion. Cells are monitored by assaying the media of transfected cells beginning at 2 days post dsRNA administration and every other day thereafter for a period of three weeks. The Auszyme ELISA, commercially available from Abbott Labs (Abbott. Park, Ill.), is used to detect surface Ag (sAg). sAg is measured since surface Ag is associated not only with viral replication but also with RNA polymerase II initiated transcription of the surface Ag cistron in the transfected infectious HBV clone. Since surface Ag synthesis can continue in the absence of HBV replication, it is important to down-regulate not only viral replication but also replication-independent synthesis of sAg. Secretion of virion particles containing encapsidated HBV genomic DNA is also measured. Loss of virion particles containing encapsidated DNA is indicative of a loss of HBV replication.

Analysis of virion secretion involves a technique that discriminates between naked, immature core particles and enveloped infectious HBV virions [7]. Briefly, pelleted viral particles from the media of cultured cells are subjected to Proteinase K digestion to degrade the core proteins. Following inactivation of Proteinase K, the sample is incubated with RQ1 DNase (Promega, Madison, Wis.) to degrade the DNA liberated from core particles. The sample is digested again with Proteinase K in the presence of SDS to inactivate the DNase as well as to disrupt and degrade the infectious enveloped virion particle. DNA is then purified by phenol/chloroform extraction and ethanol precipitated. HBV specific DNA is detected by gel electrophoresis followed by Southern Blot analysis.

Predicted Results indicate a 70-90% decrease in both sAg and viral particle secretion in the media of cells transfected with the HBV plasmid, T7 RNA polymerase expression plasmid and experimental plasmid relative to cells transfected with only the HBV plasmid T7 RNA polymerase expression plasmid and filler DNA. The multi-compartment eukaryotic expression system of the invention is also expected to produce a greater inhibition of HBV relative to a control plasmid expressing Sequence A from either a single T7 cistron or from a single UP6 cistron.

### Example 3

A Multi-Compartment Eukaryotic Expression Vector Encoding Two HBV Hairpin RNAs Shows Efficacy In Vivo.

The experiment described below utilizes hydrodynamic delivery as a method to co-deliver replication competent HBVayw plasmid together with the multi-compartment eukaryotic expression vector described in Example 1, and shown in FIG. 7, which encodes two HBV hairpin RNA molecules, each from two different cistrons with subcompartment-distinct promoters, and the T7 RNA polymerase expression plasmid of Example 1. Hydrodynamic delivery is ideal for this experiment because it results in efficient delivery of nucleic acid to the liver [8]. Combination of the dsRNA effector plasmid and replication competent HBV plasmid into the same formulation increases the likelihood that all plasmids are taken up by the same cells. Because expressed effector dsRNA are present in the majority of cells bearing the replicating HBV plasmid, observed results can be attributed to the performance of the effector plasmid rather than to differences in delivery efficiencies. This experiment demonstrates only that a particular construct is efficacious in an infected liver. Formulation and delivery are not addressed by this example. Formulation, dosing and delivery information of the eiRNA vector (expressed interfering dsRNA) are as described elsewhere herein, as well as in in vivo Example 4 in which transgenic mice are used.

Experimental procedure (in vivo): Control B10.D2 mice are hydrodynamically injected with an infectious molecular clone of HBV (ayw subtype) consisting of a terminally redundant viral genome that is capable of transcribing all of the viral RNAs and producing infectious virus [4-6]. Following internalization into hepatocytes and nuclear localization, transcription of HBVayw plasmid from several viral promoters has been shown to initiate a cascade of events that mirror HBV replication [4]. These events include translation of transcribed viral mRNAs, packaging of transcribed pregenomic RNA into core particles, reverse transcription of pregenomic RNA, and assembly and secretion of virions and HBsAg particles into the sera of injected animals. Animals are injected with four doses of the HBV replicon plasmid (1 μg, 3 μg, 5 μg and 10 μg). These doses are chosen because they represent non-saturating doses capable of eliciting detectable expression of a reporter plasmid following hydrodynamic delivery. Animals are co-injected with a 7-16 μg dose of the multi-compartment dsRNA expression vector (eiRNA) (FIG. 7) and 3 μg T7 RNA polymerase expression plasmid such that animals in each group receive a total DNA dose of 20 μg. For example in mice receiving the 3 μg dose of the HBV replicon plus 3 μg of the T7 RNA polymerase expression plasmid, 14 μg of the eiRNA vector(s) is injected for a total of 20 μg injected DNA. The amount of this eiRNA vector dose is therefore dependent upon the dose of HBV plasmid used. Control animals are injected with the HBV replicon and the T7 RNA polymerase expression plasmid but not with the eiRNA vector. Control mice are instead co-injected with an inert filler DNA, pGL3-basic (Promega, Madison, Wis.), such that the total amount of DNA in the formulation is maintained at 20 μg.

Liver samples are taken from injected animals on day 1 following injection and analyzed for the presence of HBV RNA. This time point has been selected based on published results from Dr. Chisari's laboratory which detail the kinetics of HBVayw plasmid replication in mice following hydrodynamic delivery and demonstrate that peak RNA expression occurs in the liver on day 1 following hydrodynamic delivery [4]. The presence of HBV RNA in liver samples is ascertained by Northern blot analysis. Liver tissue will be evaluated for the down-regulation of HBV RNA expression. In addition, serum will be collected from day 4 mice for measurement of HBsAg and DNA-containing viral particles. Assays will be carried out as described for the cell culture replicon experiment (Example 1) and as in Yang et al., [4]. Each vector and control group will be comprised of 2 sets of animals, each set corresponding to a collection time point. There are 5 animals is each set.

Predicted Results: mice that are injected with the HBV replicon and the eiRNA constructs have decreased HBV-specific RNA, and HBsAg and HBV viral particles as compared to the control animals. In individual animals, decreases range from about 70% to near 100%.

### Example 4

Transgenic mouse studies: Background. The HBV transgenic mouse model developed in Dr. Chisari's laboratory is used. [9-10]. These mice replicate appreciable amounts of HBV DNA and have demonstrated their utility as an antiviral screen that is a predictor of human efficacy [11]. These animals are also ideal in that they are a model for HBV-integrant-mediated expression of antigen and thus can serve as a model not only for viral replication but also for RT-independent expression of antigen. This is important as we are interested in targeting not only viral replication but integrant-mediated antigen expression as well. These experiments differ from the hydrodynamic delivery experiments in that the effector plasmids are administered to animals using clinically relevant nucleic acid delivery methods. Effectiveness in this model demonstrates efficient delivery of the effector plasmids to mouse hepatocytes.

Experiment. Mice described in reference [9] will be injected intravenously (IV) with a formulation containing the vectors described in the hydrodynamic delivery example (Example 3).

Formulation of DNA to be injected. DNA is formulated with trilactosyl spermine and cholesteryl spermine as described in U.S. Prov. 60/378,191 filed May 6, 2002, and PCT/US03/14288, filed May 6, 2003 (Satishchandran). Briefly, three formulations are made, all using a charge ratio of 1.2 (positive to negative charge). However, formulations with charge ratios between 0.8 and 1.2, inclusive, are all expected to exhibit efficacy. The cited patent applications teach compositions that include a nucleic acid, an endosomolytic spermine that includes a cholesterol or fatty acid, and a targeting spermine that includes a ligand for a cell surface molecule. The ratio of positive to negative charge of the composition is between 0.5 and 1.5, inclusive; the endosomolytic spermine constitutes at least 20% of the spermine-containing molecules in the composition; and the targeting spermine constitutes at least 10% of the spermine-containing molecules in the composition. Desirably, the ratio of positive to negative charge is between 0.8 and 1.2, inclusive, such as between 0.8 and 0.9, inclusive. The targeting spermine is designed to localize the composition to a particular cell or tissue of interest. The endosomolytic spermine disrupts the endosomal vesicle the encapsulates the composition during endocytosis, facilitating release of the nucleic acid from the endosomal vesicle and into the cytoplasm or nucleus of the cell.

The DNA starting stock solution for each plasmid has a concentration of 4 mg/ml. The two plasmid stock solutions are mixed together in equal amounts such that each plasmid is at 2 mg/ml. This plasmid mixture is used for the final formulating. Formulation is as described in U.S. Prov. 60/378,191 filed May 6, 2002, and PCT/US03/14288, filed May 6, 2003 (Satishchandran). Formulation A) 35% trilactosyl spermine, 65% cholesteryl spermine; Formulation B) 50% trilactosyl spermine, 50% cholesteryl spermine; and Formulation C) 80% trilactosysl spermine, 20% cholesteryl spermine. All resultant formulations now contain each plasmid at 1 mg/ml.

Mice are injected IV with 100 μl formulated DNA. One group of mice receives Formulation A, a second group receives Formulation B, and a third group receives Formulation C. Three groups of control mice are similarly injected with the same formulations containing a control DNA, pGL3Basic (Promega, Madison Wis.), Formulations D, E and F. Injections are carried out once a day for four consecutive days. Injecting for only 1-3 days is efficacious, however, more robust efficacy is expected with a four day injection protocol.

Following administration, HBV RNA and serum levels of HBsAg and DNA containing viral particles will be quantitated on days 5 and 9 post first injection. All analyses will be as described for the hydrodynamic delivery studies.

Expected Results: HBV-specific RNA levels, HBsAg and viral containing DNA particles will be decreased in the Formulation A, B and C groups relative to controls.

### Example 5

A Vector Encoding a Single HBV-Derived Antisense RNA Under the Control of Dual Promoters

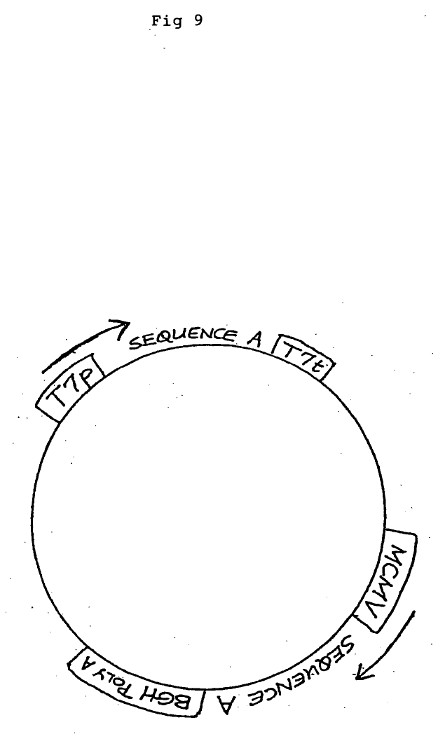

A multi-compartment eukaryotic plasmid expression vector is constructed which encodes a single HBV-specific antisense RNA, under the control of two different promoters. The promoters which direct transcription of the antisense RNA are the T7 promoter (described in Example 1) and the MCMV immediate early promoter (GenBank Accession # L06570). The transcription units (cistrons) are arranged such that there are two cistrons placed in two separate locations (FIG. 9).

Vector description: The HBV-specific RNA (Sequence A) coordinates map to 2600-2990 of Gen Bank Accession #s V01460 and J02203. The sequence is cloned into a plasmid vector such that it is situated at two loci in the plasmid as depicted in FIG. 9; The sequence is positioned in such a way relative to the directing promoter that only the antisense RNA (relative to the HBV mRNAs) strand is transcribed. One sequence is under the transcriptional control of the T7 promoter (a cytoplasmic promoter when T7 RNA polymerase is present or provided) and the other is under the transcriptional control of the MCMV IE promoter (a nuclear, non-nucleolar, RNA pol II promoter). The T7 terminator is positioned at the end of the T7 cistron as indicated in FIG. 9 and a bovine growth hormone (BGH) poly A site is positioned at the end of the MCMV cistron. A BGH poly A site is available on a number of commercially available vectors such as pVAX1 from Invitrogen (Carlsbad, Calif.) and can readily be generated by PCR amplification, for example, and can easily be performed by one skilled in the art. A poly A site was included in this example to enable efficient cytoplasmic transport but can be replaced by one or more constitutive transport elements, or CTE [12], or can be omitted. (If desired, one or more constitutive transport element (CTE) sequences can be added to enable cytoplasmic transport of the different effector RNA molecules (e.g., mRNAs, hairpin or duplex dsRNAs) that are made in the nucleus by RNA pol II. A CTE can be used instead of and/or in addition to an intron and/or polyA sequence to facilitate transport. A desirable location for the CTE is near the 3′ end of the RNA molecules. If desired, multiple CTE sequences (e.g., 2, 3, 4, 5, 6, or more sequences can be used). A preferred CTE is from the Mason-Pfizer Monkey Virus, as taught in U.S. Pat. Nos. 5,880,276 and 5,585,263, incorporated herein by reference.) This vector is assessed in an HBV replicon model. Cloning is performed using standard techniques or, if desired, directional ligation cloning, CRC, can be used [3]. (See the teaching of U.S. Pat. No. 6,143,527, “Chain reaction cloning using a bridging oligonucleotide and DNA ligase”, Pachuk et al., incorporated herein by reference.)

These vectors are assessed in an HBV replicon model as described in Example 1. For this experiment, a T7 RNA polymerase expression plasmid as described above is co-transfected with the experimental plasmid so as to provide a source of T7 RNA polymerase.

HBV Replicon Model: Silencing HBV Replication and Expression in a Replication Competent Cell Culture Model.

Brief description of cell culture model: A human liver derived cell line such as the Huh7 cell line is transfected with an infectious molecular clone of HBV consisting of a terminally redundant viral genome that is capable of transcribing all of the viral RNAs and producing infectious virus [4-6]. The replicon used in these studies is derived from the virus sequence found in Gen Bank Accession #s V01460 and J02203. Following internalization into hepatocytes and nuclear localization, transcription of the infectious HBV plasmid from several viral promoters has been shown to initiate a cascade of events that mirror HBV replication. These events include translation of transcribed viral mRNAs, packaging of transcribed pregenomic RNA into core particles, reverse transcription of pregenomic RNA, and assembly and secretion of virions and HBsAg particles into the media of transfected cells. This transfection model reproduces most aspects of HBV replication within infected liver cells and is therefore a good cell culture model with which to look at silencing of HBV expression and replication.

In this model, cells are co-transfected with the infectious molecular clone of HBV and the effector RNA constructs to be evaluated. The cells are then monitored for loss of HBV expression and replication as described below.

Experimental Procedure: Transfection. Huh7 cells are seeded into six-well plates such that they are between 80-90% confluency at the time of transfection. All transfections are performed using Lipofectamine™ (Invitrogen) according to the manufacturer's directions. In this experiment, cells are transfected with 50 ng of the infectious HBV plasmid, 1 μg of a T7 RNA polymerase expression plasmid (description of plasmid in Example 1) and 1.5 μg of the experimental plasmid depicted in FIG. 9. Control cells are transfected with 50 ng of the HBV plasmid and 1 μg of the T7 RNA polymerase expression plasmid. An inert filler DNA, pGL3-basic (Promega, Madison WI), is added to all transfections to bring total DNA/transfection up to 2.5 μg DNA.

Monitoring cells for loss of HBV expression. Following transfection, cells are monitored for the loss or reduction in HBV expression and replication by measuring HBsAg secretion and DNA-containing viral particle secretion. Cells are monitored by assaying the media of transfected cells beginning at 2 days post dsRNA administration and every other day thereafter for a period of three weeks. The Auszyme ELISA, commercially available from Abbott Labs (Abbott Park, IL), is used to detect surface Ag (sAg). sAg is measured since surface Ag is associated not only with viral replication but also with RNA polymerase II initiated transcription of the surface Ag cistron in the transfected infectious HBV clone. Since surface Ag synthesis can continue in the absence of HBV replication, it is important to down-regulate not only viral replication but also replication-independent synthesis of sAg. Secretion of virion particles containing encapsidated HBV genomic DNA is also measured. Loss of virion particles containing encapsidated DNA is indicative of a loss of HBV replication.

Analysis of virion secretion involves a technique that discriminates between naked, immature core particles and enveloped infectious HBV virions [7]. Briefly, pelleted viral particles from the media of cultured cells are subjected to Proteinase K digestion to degrade the core proteins. Following inactivation of Proteinase K, the sample is incubated with RQ1 DNase (Promega, Madison, Wis.) to degrade the DNA liberated from core particles. The sample is digested again with Proteinase K in the presence of SDS to inactivate the DNase as well as to disrupt and degrade the infectious enveloped virion particle. DNA is then purified by phenol/chloroform extraction and ethanol precipitated. HBV specific DNA is detected by gel electrophoresis followed by Southern Blot analysis.

Expected Results indicate a decrease in both sAg and viral particle secretion in the media of cells transfected with the HBV plasmid, T7 RNA polymerase expression plasmid and experimental plasmid relative to cells transfected with only the HBV plasmid T7 RNA polymerase expression plasmid and filler DNA.

### Example 6

A Vector Utilizing Three Promoters: a Nuclear Promoter to Drive Expression of T7 RNA Polymerase and Two T7 Promoters to Drive Expression of a Sense and Antisense RNA, Respectively.

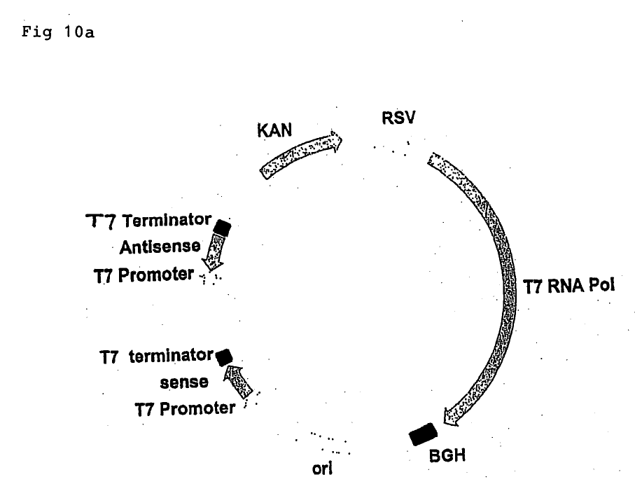

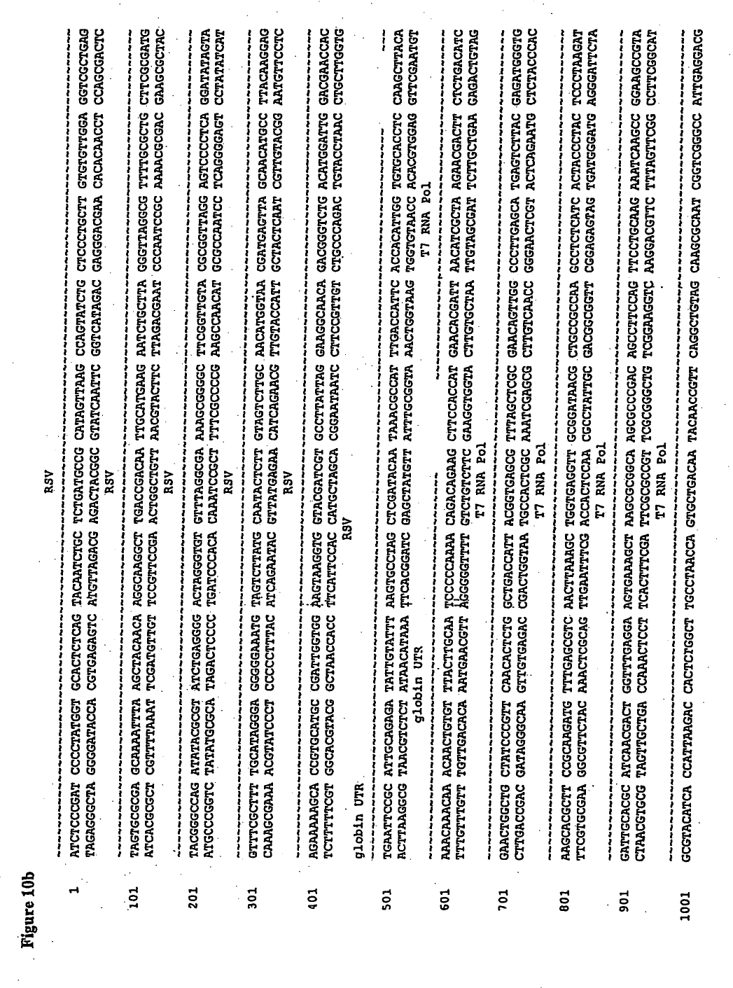

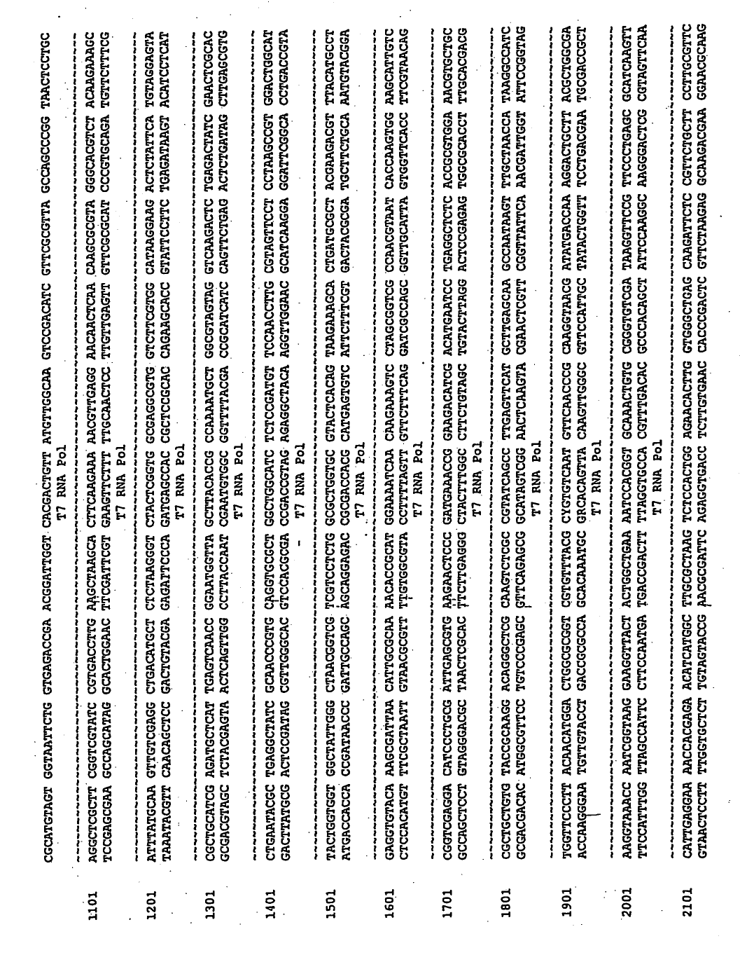

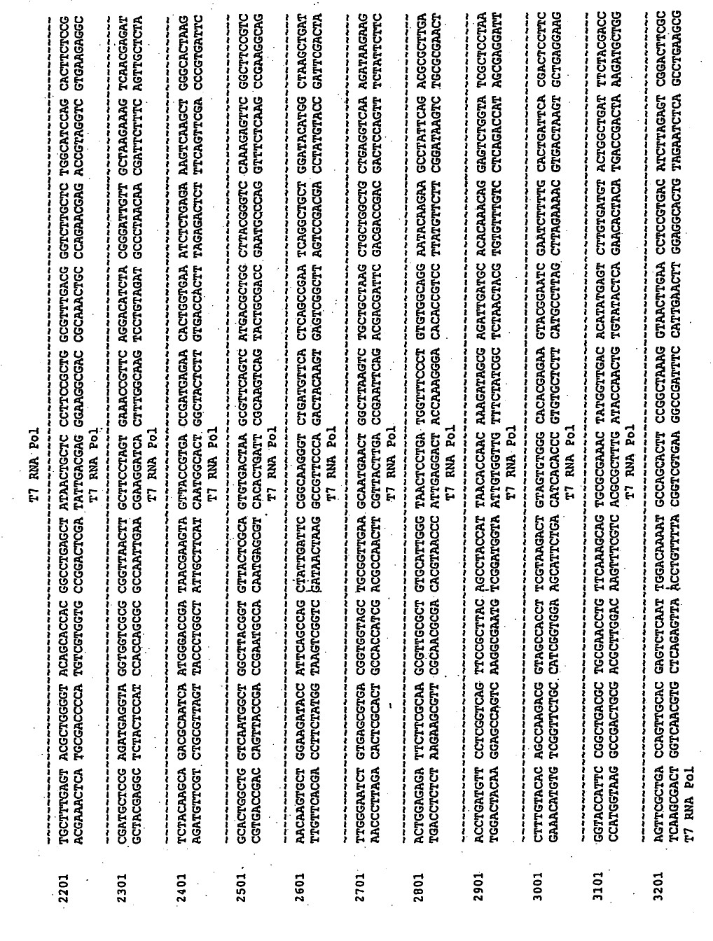

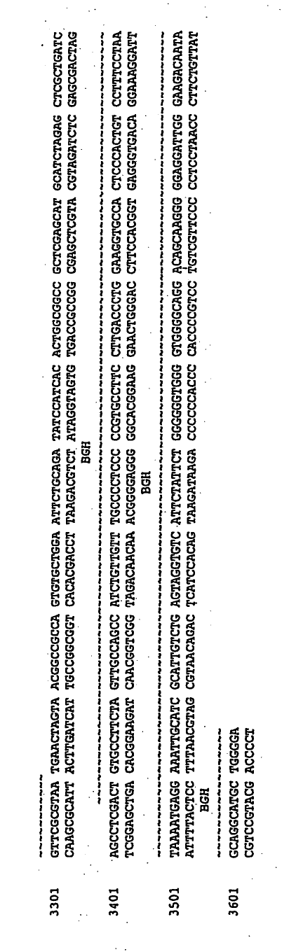

A plasmid (see FIG. 10a) is constructed which encodes the sense and antisense strands of an HBV-specific RNA sequence. The expression of the sense and antisense RNAs is under the control of two T7 promoters, located in two separate cistrons. The T7 RNA polymerase gene is also encoded on the same vector under the control of an RNA pol II promoter, the RSV promoter. In the cell RNA pol II transcribes the T7 RNA polymerase gene from the RSV promoter in the nucleus (non-nucleolus). The T7 RNA polymerase mRNA is then transported to and translated in the cytoplasm where it is active and transcribes the T7 driven sense and antisense RNAs from cytoplasmically localized plasmids. The sense and antisense RNAs can then basepair with each other to form duplex dsRNA.

Vector description: The HBV-specific RNA (Sequence A) coordinates map to 2600-2990 of accession #s V01460 and J02203. The sequence is cloned into a plasmid vector such that it is situated at two loci in the plasmid as depicted in FIG. 10a. The sequence is positioned in such a way relative to the directing promoter that antisense RNA (relative to the HBV mRNAs) is transcribed by one T7 promoter and sense RNA is transcribed by the other. The T7 terminator is positioned at the ends of each T7 cistron. The sequence of the T7 RNA polymerase expression cassette is shown in FIG. 10b (SEQ ID NO:3) and is comprised of the RSV promoter, the 5′ UTR, the T7 RNA polymerase coding region and the BGH polyadenylation site. A picture of the vector is depicted in FIG. 10a. This vector is assessed in an HBV replicon model described below. Cloning is performed using standard techniques or, if desired, directional ligation cloning, CRC, can be used [3]. (See the teaching of U.S. Pat. No. 6,143,527, “Chain reaction cloning using a bridging oligonucleotide and DNA ligase”, Pachuk et al., incorporated herein by reference.)

HBV Replicon Model: Silencing HBV Replication and Expression in a Replication Competent Cell Culture Model.

Brief description of cell culture model: A human liver derived cell line such as the Huh7 cell line is transfected with an infectious molecular clone of HBV consisting of a terminally redundant viral genome that is capable of transcribing all of the viral RNAs and producing infectious virus [4-6]. The replicon used in these studies is derived from the virus sequence found in Gen Bank Accession #s V01460 and J02203. Following internalization into hepatocytes and nuclear localization, transcription of the infectious HBV plasmid from several viral promoters has been shown to initiate a cascade of events that mirror HBV replication. These events include translation of transcribed viral mRNAs, packaging of transcribed pregenomic RNA into core particles, reverse transcription of pregenomic RNA, and assembly and secretion of virions and HBsAg particles into the media of transfected cells. This transfection model reproduces most aspects of HBV replication within infected liver cells and is therefore a good cell culture model with which to look at silencing of HBV expression and replication.

In this model, cells are co-transfected with the infectious molecular clone of HBV and the effector RNA constructs to be evaluated. The cells are then monitored for loss of HBV expression and replication as described below.

Experimental Procedure: Transfection. Huh7 cells are seeded into six-well plates such that they are between 80-90% confluency at the time of transfection. All transfections are performed using Lipofectamine™ (Invitrogen) according to the manufacturer's directions. In this experiment, cells are transfected with 50 ng of the infectious HBV plasmid and 2.5 μg of the experimental plasmid depicted in FIG. 10a. Control cells are transfected with 50 ng of the HBV plasmid. An inert filler DNA, pGL3-basic (Promega, Madison Wis.), is added to all transfections to bring total DNA/transfection up to 2.5 μg DNA.

Monitoring cells for loss of HBV expression. Following transfection, cells are monitored for the loss or reduction in HBV expression and replication by measuring HBsAg secretion and DNA-containing viral particle secretion. Cells are monitored by assaying the media of transfected cells beginning at 2 days post dsRNA administration and every other day thereafter for a period of three weeks. The Auszyme ELISA, commercially available from Abbott Labs (Abbott Park, Ill.), is used to detect surface Ag (sAg). sAg is measured since surface Ag is associated not only with viral replication but also with RNA polymerase II initiated transcription of the surface Ag cistron in the transfected infectious HBV clone. Since surface Ag synthesis can continue in the absence of HBV replication, it is important to down-regulate not only viral replication but also replication-independent synthesis of sAg. Secretion of virion particles containing encapsidated HBV genomic DNA is also measured. Loss of virion particles containing encapsidated DNA is indicative of a loss of HBV replication.

Analysis of virion secretion involves a technique that discriminates between naked, immature core particles and enveloped infectious HBV virions [7]. Briefly, pelleted viral particles from the media of cultured cells are subjected to Proteinase K digestion to degrade the core proteins. Following inactivation of Proteinase K, the sample is incubated with RQ1 DNase (Promega, Madison, Wis.) to degrade the DNA liberated from core particles. The sample is digested again with Proteinase K in the presence of SDS to inactivate the DNase as well as to disrupt and degrade the infectious enveloped virion particle. DNA is then purified by phenol/chloroform extraction and ethanol precipitated. HBV specific DNA is detected by gel electrophoresis followed by Southern Blot analysis.

Expected Results indicate a decrease in both HBsAg and viral particle secretion in the media of cells transfected with the HBV plasmid, T7 RNA polymerase expression plasmid, and the experimental plasmid, relative to cells transfected with only the HBV plasmid, T7 RNA polymerase expression plasmid, and filler DNA.

### Example 7

Dual/Embedded Promoter Expression System: a Multi-Compartment Eukaryotic Expression Vector Utilizing an Embedded Promoter System: a T7 Promoter Embedded within the MCMV Promoter.

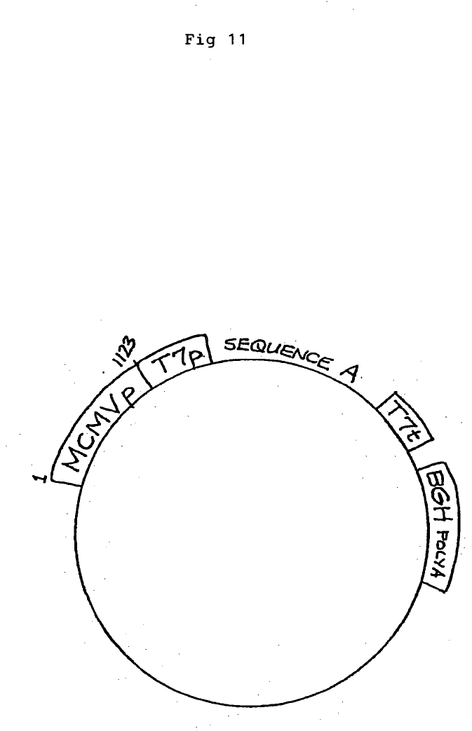

A plasmid is constructed which encodes an HBV-specific antisense RNA sequence. The expression of the antisense RNA is under the control of an embedded promoter (FIG. 11). This expression plasmid is delivered together with a T7 RNA polymerase expression vector, as described in Example 1. To create the dual/embedded promoter, the last 17 nucleotides at the 3′ end of the MCMV promoter (GenBank Accession # X03922) are deleted and replaced with the T7 promoter as depicted in FIG. 11. In the cytoplasm, the embedded promoter vector expresses the antisense RNA from the T7 promoter, whereas vectors localized to the nucleus will express the antisense RNA from the MCMV promoter, which is an RNA pol II promoter active in the nucleus.

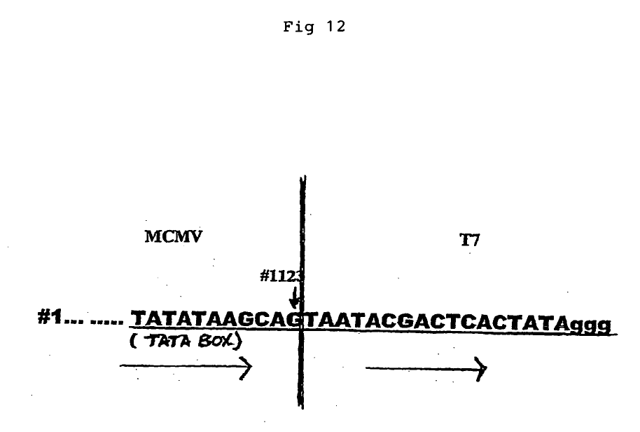

Vector description: The HBV-specific RNA (Sequence A) coordinates map to 2600-2990 of accession #s V01460 and J02203. The sequence is cloned into a plasmid vector in an orientation such that the antisense strand is transcribed with respect to the promoters. The MCMV promoter contains sequences mapping to coordinates 1-1123 inclusive of GenBank accession # X03922. The T7 promoter (as described in Example 1) is immediately juxtaposed to this sequence such that the nucleotide immediately following the MCMV nucleotide mapping to coordinate 1123 of GenBank accession # X03922 is the first nucleotide of the T7 promoter (FIG. 12). The T7 terminator and BGH polyadenylation signal are positioned in the vector as indicated in FIG. 11.

This vector is assessed in an HBV replicon model. Cloning is performed using standard techniques or, if desired, directional ligation cloning, CRC, can be used [3]. (See the teaching of U.S. Pat. No. 6,143,527, “Chain reaction cloning using a bridging oligonucleotide and DNA ligase”, Pachuk et al, incorporated herein by reference.)

HBV Replicon Model: Silencing HBV Replication and Expression in a Replication Competent Cell Culture Model.

Brief description of cell culture model: A human liver derived cell line such as the Huh7 cell line is transfected with an infectious molecular clone of HBV consisting of a terminally redundant viral genome that is capable of transcribing all of the viral RNAs and producing infectious virus [4-6]. The replicon used in these studies is derived from the virus sequence found in Gen Bank Accession #s V01460 and J02203. Following internalization into hepatocytes and nuclear localization, transcription of the infectious HBV plasmid from several viral promoters has been shown to initiate a cascade of events that mirror HBV replication. These events include translation of transcribed viral mRNAs, packaging of transcribed pregenomic RNA into core particles, reverse transcription of pregenomic RNA, and assembly and secretion of virions and HBsAg particles into the media of transfected cells. This transfection model reproduces most aspects of HBV replication within infected liver cells and is therefore a good cell culture model with which to look at silencing of HBV expression and replication.

In this model, cells are co-transfected with the infectious molecular clone of HBV and the effector RNA constructs to be evaluated. The cells are then monitored for loss of HBV expression and replication as described below.

Experimental Procedure: Transfection. Huh7 cells are seeded into six-well plates such that they are between 80-90% confluency at the time of transfection. All transfections are performed using Lipofectamine™ (Invitrogen) according to the manufacturer's directions. In this experiment, cells are transfected with 50 ng of the infectious HBV plasmid and 2.5 μg of the experimental plasmid depicted in FIG. 11. Control cells are transfected with 50 ng of the HBV plasmid. An inert filler DNA, pGL3-basic (Promega, Madison Wis.), is added to all transfections to bring total DNA/transfection up to 2.5 μg DNA.

Monitoring cells for loss of HBV expression. Following transfection, cells are monitored for the loss or reduction in HBV expression and replication by measuring HBsAg secretion and DNA-containing viral particle secretion. Cells are monitored by assaying the media of transfected cells beginning at 2 days post dsRNA administration and every other day thereafter for a period of three weeks. The Auszyme ELISA, commercially available from Abbott Labs (Abbott Park, Ill.), is used to detect HBV surface Ag (sAg). HBsAg is measured since surface Ag is associated not only with viral replication but also with RNA polymerase II initiated transcription of the surface Ag cistron in the transfected infectious HBV clone. Since surface Ag synthesis can continue in the absence of HBV replication, it is important to down-regulate not only viral replication but also replication-independent synthesis of sAg. Secretion of virion particles containing encapsidated HBV genomic DNA is also measured. Loss of virion particles containing encapsidated DNA is indicative of a loss of HBV replication.

Analysis of virion secretion involves a technique that discriminates between naked, immature core particles and enveloped infectious HBV virions [7]. Briefly, pelleted viral particles from the media of cultured cells are subjected to Proteinase K digestion to degrade the core proteins. Following inactivation of Proteinase K, the sample is incubated with RQ1 DNase (Promega, Madison, Wis.) to degrade the DNA liberated from core particles. The sample is digested again with Proteinase K in the presence of SDS to inactivate the DNase as well as to disrupt and degrade the infectious enveloped virion particle. DNA is then purified by phenol/chloroform extraction and ethanol precipitated. HBV specific DNA is detected by gel electrophoresis followed by Southern Blot analysis.

Expected Results indicate a decrease in both sAg and viral particle secretion in the media of cells transfected with the HBV plasmid, T7 RNA polymerase expression plasmid and experimental plasmid relative to cells transfected with only the HBV plasmid T7 RNA polymerase expression plasmid and filler DNA.

### Example 8

Use of a Pol I and Pol II Promoter in the Same Vector to Express Two Forms of a dsRNA Molecule for Optimal Gene Silencing Effects.

When using gene silencing vectors in cell culture or in small animal experiments, it is usually possible, particularly in cell culture, to transfect the vectors in sufficient quantity such that the nucleus of many cells can take up at least several copies of this vector and allow the cell culture as a whole to express the dsRNA molecule with high efficiency. On the other hand, when pharmacologically-suitable doses of these vectors are to be used in a large animal for uptake by complex organs, such as the liver or lung of a human, the transfer of vector to the cell nuclei will be much less efficient. Most cells of said tissue will take up no vector while others may only contain one or two copies of this vector. It is in this limiting situation that the present invention, comprising multi-compartment expression vectors, is especially advantageous, engendering distinct pharmacological advantages.

In this example, RNA polymerase II and polymerase I promoters, typically active in different physical nuclear subcompartments (nucleoplasm and nucleolus, respectively), are both used to encode the expression of a shRNA molecule from a vector of the instant invention (Vector “A”). For the purpose of comparison, and to serve as experimental controls, two other vectors are used: one containing only the pol II/shRNA expresssion cassette (Vector “B”) and one containing only the pol I/shRNA expression cassette (Vector “C”). Each vector contains a non-interfering chemical label incorporated via nucleotide analogues during synthesis (such as Cy5, Cy3, digoxygenin, bromodeoxyuridine, etc.) which is used to visualize the location of each vector in the cell nucleus by electron microscopy after transfection.

The use of polymerase II promoters to express shRNA molecules has precedent in natural cellular mechanisms involving microRNAs (mRNA). While mRNAs and engineered shRNAs both contain dsRNA in hairpin configurations, the generation and activation pathways engendered by vector-mediated (engineered) shRNA differ in important ways from those used by endogenous mRNAs. For example, mRNAs are all synthesized as long (e.g. up to several kilobases) primary precursor transcripts (pri-mRNAs) by RNA polymerase II, and not by the RNA polymerase III enzymes commonly employed in shRNA expression vectors. Also, both shRNAs and mRNAs share cytoplasmic processing steps via the RNAse III enzyme known as Dicer but mRNAs require an additional processing step catalyzed by another RNAse III activity known as Drosha, which is present only in cell nuclei (e.g. see Lee et al., MicroRNA Maturation: stepwise processing and subcellular localization, EMBO Journal, v.21, pp. 4663-4670, 2002).

For this example, hairpins are designed to silence a hepatitis B virus protein, sAg (surface antigen). In the experimental system used in this example, Huh7 (human hepatoma cell line) cells are engineered to stably express a copy of the hepatitis B surface antigen gene, and produce a relatively constant level of sAg protein and RNA.

Using one of any commonly available vector backbones (e.g., see the catalog of Invitrogen Corp. or Stratagene Inc.), an RNA pol I shRNA expression vector is made by cloning the shRNA of interest downstream of the transciption start site of a pol I promoter, preferably one in which all the elements needed for promoter function are located upstream of the transcription start site in the native pol I gene. The shRNA of interest, named HBV-shRNA-1907, comprises SEQ ID NO:4. The first 21 bases of SEQ ID NO:4 are identical to the sense sequence of HBV mRNA from position 1907 to 1927 in the HBV genome, strain AYW (numbered according to the complement strand given in GenBank® Accession No. V01460). This sequence is followed by 9 bases (i.e., AGAGAACTT) representing the loop portion of the shRNA, followed by 21 bases of the reverse complementary sequence to the first 21 bases. (It will be understood that the loop sequence serves only to join the complementary sequences which form the double-stranded “stem” and therefore considerable variation in length and nucleotide sequence is acceptable within the loop region.). Preferably a short terminator element (e.g., 4, 5, or more T residues) are located 3′ of the shRNA sequence. This is Vector C.

An RNA pol II/shRNA expression cassette is made by first placing the HBV-shRNA-1907 hairpin stem sequence only (the first 21 bases of SEQ ID NO:4 and its reverse complement) into the primary micro RNA sequence of the microRNA known as miR24-2 (GenBank® Accession No. AF480559, and Mourelatos et al., miRNPs: a novel class of ribonucleoproteins containing numerous microRNAs, Genes & Development v. 16, pp. 720-728, 2002). Thus, the HBV sequence of 21 bases is inserted between positions 10 and 33 of miR24, removing the native sequence between those positions, and the 21 base reverse complement is inserted into the native miR24 hairpin at position 57 to 78 of the miR24 sequence. The new hybrid hairpin thus contains loop and flanking sequences of miR24 but the base-paired stem of the hairpin consists of the HBV sequences and flanking miR24 nucleotides of the pre miR24 RNA. In order to effect expression of this HBV hairpin in the miR24 from pol II in a manner to ensure correct processing by Drosha, the entire gene comprising the natural promoter and sequences encoding the primary miR24 transcript is cloned into an expression vector. Subsequently, using a number of restriction enzyme steps, synthetic oligonucleotides and annealing steps, the HBV/miR24 pre miRNA segment above is inserted into the miR24 full promoter construct. This is placed in the same vector backbone used to make Vector C and now constitutes Vetor B.

Vector A (the Pol II and Pol I promoter vector of the invention) is made by combining the Pol II and the Pol I expression cassettes of vectors B and C into a single plasmid using standard restriction enzyme cloning methods.

Each of the three labeled vectors is individually transfected into the Huh7/sAg antigen cells expressing the HBV RNA. Using highly sensitive conventional immunostaining techniques for quantitating HBV sAg expression, it is possible to measure the amount of sAg made in individual cells (e.g., HBV sAg Auszyme® reagents from Abbott Laboratories). In parallel, using the detection methods for DNA under conditions where cellular structure are observable, it is possible to determine where in the nucleus the plasmid expression vector is located (e.g., nucleolus or nucleoplasm).

The results are expected as follows. Cells transfected with Vector B, and which have Vector B located in the nucleoplasm, are capable of reducing the expression of HBV sAg relative to control (untransfected cells); however, those cells in which Vector B is seen to be present only in the nucleoli are not observed to decrease the expression of sAg. This observation would be consistent with data cited by Thomson et al. (Science, v.302, pp. 1399-1400, 2003, and references 7, 9 and 10 cited within) that pol II transcription may be inhibited or restricted in the physical domains of rRNA gene activity (i.e., the nucleolus).

Cells which are transfected with Vector C, and have Vector C located at sites in the nucleoplasm but distinct from the nucleoli, do not display reduced HBV sAg expression relative to mock-transfected control cells, ostensibly because the pol I promoter in this vector is not in proximity to the compartmentalized sites of pol I activity, i.e., the nucleoli. However, reduction of HBV sAg expression is observed in those cells where Vector C has localized to the nucleolus.

The observations with Vector A are in contrast to the results seen with Vectors B and C, where these vectors' effects on silencing HBV sAg expression depend on the localization of vector within the cell nucleus. When Vector A is transfected into the Huh7 cells expressing HBV sAg, the levels of HBV sAg are markedly reduced regardless of whether Vector A localizes to the nucleolus or to other sites in the nucleoplasm of the cell.
[1] Lyakhov, D. L., et al., Pausing and termination by bacteriophage T7 RNA polymerase. J. Mol. Biol. 280:201-213 (1998).
[2] Lee, N. S., et al. Expression of small interfering RNAs targeted against HIV-1 rev transcripts in human cells. Nat. Biotechnol. 20:500-505 (2002).
[3] Pachuk, C. J., et al, Chain reaction cloning: a one-step method fordirectional ligation of multiple DNA fragments. Gene 243:19-25 (2000).
[4] Yang, P. L., et al., Hydrodynamic injection of viral DNA: a mouse model of acute hepatitis B virus infection. Proc. Natl. Acad. Sci. USA 99:13825-30 (2002).
[5] Guidotti, L. G., et al., Viral clearance without destruction of infected cells during acute HBV infection. Science 284:825-9 (1999).
[6] Thimme, R., et al., CD8(+). T cells mediate viral clearance and disease pathogenesis during acute hepatitis B virus infection. J. Virol. 77:68-76 (2003).
[7] Delaney, W. E. 4th and Isom, H. C., Hepatitis B virus replication in human HepG2 cells mediated by hepatitis B virus recombinant baculovirus. Hepatology 28:1134-46 (1998).
[8] Liu, F., et al., Hydrodynamics-based transfection in animals by systemic administration of plasmid DNA. Gene Ther. 6:1258-66 (1999).
[9] Guidotti, L. G., et al., High-level hepatitis B virus replication in transgenic mice. J. Virol. 69:6158-69 (1995).
[10] Chisari, F. V., et al., A transgenic mouse model of the chronic hepatitis B surface antigen carrier state. Science 230:1157-60 (1985).
[11] Morrey, J. D, et al, Transgenic Mice as a chemotherapeutic model for hepatitis B virus infection. In:Therapies for Viral Hepatits, Eds. Schinazi, R. F., Sommadossi, J. P. and Thomas H. VC. 1998. International Medical Press, London, UK.
[12] Pasquinelli A. E., et al., The constitutive transport element (CTE) of Mason-Pfizer monkey virus (MPMV) accesses a cellular mRNA export pathway. EMBO J. 16:7500-7510 (1997).

1. A eukaryotic expression system comprising one or more expression constructs, wherein said one or more expression constructs collectively comprise at least a first and a second promoter, wherein said first and second promoters are each transcriptionally active within a different subcellular compartment of the same eukaryotic cell, and wherein said first and second promoter are each operably linked to a sequence encoding a nucleic acid molecule which is not capable of translation.

2. The expression system of claim 1, wherein said first and said second promoters are operably linked to nucleic acid sequences encoding different nucleic acid molecules.

3. The expression system of claim 1, wherein said first and said second promoters are operably linked to nucleic acid sequences encoding the same nucleic acid molecule.

4. The expression system of claim 3, wherein said first and said second promoters are both operably linked to a single copy of said nucleic acid sequence.

5. The expression system of claim 3, wherein said first and said second promoters are operably linked to different copies of said nucleic acid sequence.

6. The expression system of claim 1, wherein said subcellular compartments are selected from the cytoplasm, the mitochondria, the nucleus, the nucleolus, a functional domain within the cytoplasm, a functional domain within the nucleus and a functional domain within the nucleolus.

7. The expression system of claim 1, wherein said first and second promoters are transcriptionally active in the nucleus and cytoplasm, in the nucleus and mitochondria, in the nucleus and nucleolus, in the cytoplasm and mitochondria, in the cytoplasm and nucleolus, in the mitochondria and nucleolus, or in different functional compartments of the nucleolus.

8. The expression system of claim 1, further comprising a third promoter.

9. The expression system of claim 8, wherein said third promoter is transcriptionally active within a subcellular compartment different from said first and second promoters.

10. The expression system of claim 8, wherein said first, second and third promoters are transcriptionally active in the cytoplasm, nucleus, and nucleolus; in the cytoplasm, nucleus, and mitochondria; in the cytoplasm, nucleolus, and mitochondria; or in the mitochondria, nucleolus, and mitochondria.

11. The expression system of claim 1, wherein the promoters are selected from RNA pol I, RNA pol III, RNA pol II, T7, SP6, SP3, an RNA viral promoter, a mitochondrial heavy chain promoter, a mitochondrial light chain promoter, an RNA pol III Type 2 promoter and an RNA pol III Type 3 promoter.

12. (canceled)

13. The expression system of claim 1, wherein the first and second promoters are located on two different expression constructs.

14. The expression system of claim 1, wherein the eukaryotic cell is a mammalian cell.

15. The expression system of claim 14, wherein the mammalian cell is a human cell.

16. The expression system of claim 1, wherein said expression constructs are delivered to the eukaryotic cell from the same composition.

17. The expression system of claim 1, wherein said expression constructs are delivered to the eukaryotic cell from two different compositions.

18. The expression system of claim 17, wherein the two compositions are delivered to the eukaryotic cell at the same time.

19. The expression system of claim 17, wherein the two compositions are delivered to the eukaryotic cell at different times.

20. The expression system of any of claims 1 to 19 wherein said first promoter or said second promoter is operably linked to a nucleic acid sequence which encodes a molecule capable of modulating the expression of a target gene.

21. The expression system of any of claims 1 to 19 wherein said first promoter and said second promoter are each operably linked to a nucleic acid sequence which encodes a molecule capable of modulating the expression of a target gene.

22. The expression system of claim 20, wherein said target gene is viral gene.

23. The expression system of claim 21, wherein said target gene is a viral gene.

24. A method of delivering to a eukaryotic cell a molecule of interest, comprising delivering to said eukaryotic cell the one or more expression constructs of claim 1, wherein at least one of said expression constructs comprises a nucleic acid sequence encoding at least a portion of the molecule of interest.

25. The method of claim 24, wherein said one or more expression constructs are delivered to the eukaryotic cell from different compositions.

26. The method of claim 24, wherein said one or more expression constructs are delivered to the eukaryotic cell from the same composition.

27. The method of claim 25, wherein said molecule of interest is capable of modulating the expression of a target gene.

28. The method of claim 26, wherein said molecule of interest is capable of modulating the expression of a target gene.

29. The method of claim 27, wherein said target gene is a viral gene.

30. The method of claim 28, wherein said target gene is a viral gene.

31. The method of claim 29, wherein said eukaryotic cell is a mammalian cell.

32. The method of claim 30, wherein said eukaryotic cell is a mammalian cell.

33. The method of claim 31, wherein said mammalian cell is a human cell.

34. The method of claim 32, wherein said mammalian cell is a human cell.

35. An expression construct comprising at least a first and a second promoter, wherein said first and second promoters are each transcriptionally active within a different subcellular compartment of the same eukaryotic cell, and wherein said first and second promoter are each operably linked to a sequence encoding a nucleic acid molecule which is not capable of translation.

36. A mammalian cell comprising the construct of claim 35.

37. A human cell comprising the construct of claim 35.

38. The expression system of claim 1, wherein at least one of said first and second promoters is operably linked to nucleic acid sequence encoding a dsRNA molecule.

39. The expression system of claim 38, wherein both of said first and second promoters are operably linked to a nucleic acid sequence encoding a dsRNA molecule.

40. The expression system of claim 39, wherein said first and second promoters are operably linked to a nucleic acid sequence encoding an identical dsRNA molecule.

41. The expression system of claim 1, wherein said first and second promoters are activated by endogenous RNA polymerases.

42. The expression system of claim 12, wherein said expression construct is a plasmid.

43. The expression system of claim 1, wherein said first and second promoters are selected from the group consisting of: an RNA polymerase I promoter and a RNA polymerase II promoter; an RNA polymerase I promoter and a RNA polymerase III promoter; an RNA polymerase I promoter and a mitochondrial promoter; an RNA polymerase I promoter and a bacteriophage promoter; an RNA polymerase II promoter and a RNA polymerase III promoter; an RNA polymerase II promoter and a RNA polymerase III type 3 promoter; an RNA polymerase II promoter and a mitochondrial promoter; an RNA polymerase II promoter and a bacteriophage promoter; an RNA polymerase III promoter and a mitochondrial promoter; an RNA polymerase III promoter and a bacteriophage promoter; and an RNA polymerase III type 3 promoter and a bacteriophage promoter.

44. The expression system of claim 1, wherein said first and second promoters are selected from the group consisting of: an RNA polymerase I promoter and a RNA polymerase II promoter; an RNA polymerase I promoter and a RNA polymerase III promoter; an RNA polymerase I promoter and a mitochondrial promoter; an RNA polymerase II promoter and a RNA polymerase III promoter; an RNA polymerase II promoter and a RNA polymerase III type 3 promoter; an RNA polymerase II promoter and a mitochondrial promoter; an RNA polymerase III promoter and a mitochondrial promoter; and an RNA polymerase III type 3 promoter and a mitochondrial promoter.

45. The expression construct of claim 35, wherein said first and second promoters are selected from the group consisting of: an RNA polymerase I promoter and a RNA polymerase II promoter; an RNA polymerase I promoter and a RNA polymerase III promoter; an RNA polymerase I promoter and a mitochondrial promoter; an RNA polymerase II promoter and a RNA polymerase III promoter; an RNA polymerase II promoter and a RNA polymerase III type 3 promoter; an RNA polymerase II promoter and a mitochondrial promoter; an RNA polymerase III promoter and a mitochondrial promoter; and an RNA polymerase III type 3 promoter and a mitochondrial promoter.
